(19) United States
(12) Patent Application Publication
(10) Pub. No.: US 2025/0249124 A1
(43) Pub. Date: Aug. 7, 2025

(54) ADENO-ASSOCIATED VIRUS COMPOSITIONS HAVING INCREASED BRAIN ENRICHMENT

(71) Applicant: CAPSIDA, INC., Thousand Oaks, CA (US)

(72) Inventors: Troy E. Sandberg, Thousand Oaks, CA (US); Brandon G. Wheeler, Thousand Oaks, CA (US); Nicholas S. Goeden, Thousand Oaks, CA (US); Nicholas C. Flytzanis, Thousand Oaks, CA (US)

(21) Appl. No.: 18/857,095

(22) PCT Filed: May 5, 2023
(86) PCT No.: PCT/US2023/021131

§ 371 Date: Oct. 15, 2024

Related U.S. Application Data
(60) Provisional application No. 63/339,148, filed on May 6, 2022.

(51) Int. Cl. A61K 48/00; C07K 14/005; C12N 15/86

(52) U.S. Cl. CPC A61K 48/0041; A61K 48/0075; C07K 14/005; C12N 15/86; C12N 2750/14122; C12N 2750/14143; C12N 2750/14145; C12N 2750/14152; C12N 2800/40

(57) ABSTRACT

Described herein are compositions and kits comprising recombinant adeno-associated viruses (rAAVs) with increased transduction enrichment in the brain. The rAAV compositions described herein encapsidate a transgene, such as a therapeutic nucleic acid. Gene therapy using the rAAVs is described. Also described are methods of treating brain-related diseases and conditions.

### BACKGROUND

Recombinant adeno-associated viruses (rAAVs) are widely used as vectors for gene delivery in therapeutic applications because of their ability to transduce both dividing and non-dividing cells, their long-term persistence as episomal DNA in infected cells, and their low immunogenicity. These characteristics make them appealing for applications in therapeutic applications, such as gene therapy. However, there is a need to significantly improve the performance of existing AAV serotypes to selectively and efficiently express in distinct cell-types, upon systemic delivery to a subject. This need is especially acute when the AAV must be expressed in the brain.

### SUMMARY OF THE INVENTION

Disclosed herein are rAAVs with engineered transduction into the capsid structure through iterative rounds of selection in non-human primates (NHPs), yielding variants having increased transduction when measured in the brain.

The present invention provides rAAVs with widespread transduction to the brain.

The present invention provides, in an aspect, a peptide insertion sequence comprising or consisting of an amino-acid sequence set forth in any one of Tables 1-3, FIG. 1 and and/or Formulas I-VI.

Another aspect of the invention is a modified capsid protein wherein the AAV capsid protein, with a peptide insertion comprising or consisting of an amino-acid sequence set forth in any one of Tables 1-3, FIG. 1 and and/or Formulas I-VI, is characterized by increased brain transduction in a subject.

The present disclosure moreover includes pharmaceutical compositions comprising rAAVs with a peptide insertion comprising or consisting of an amino-acid sequence set forth in any one of Tables 1-3, FIG. 1 and and/or Formulas I-VI, and a pharmaceutically acceptable excipient.

Aspects disclosed herein provide methods of treating a disease or condition in a subject comprising administering a therapeutically effective amount of a pharmaceutical formulation comprising the AAV capsid protein or the AAV capsid of the present disclosure. In some embodiments, the disease or the condition is a disease or a condition of the brain, and brain of the subject. Relatedly, the invention includes use of the rAAVs in the manufacture of a medicament for treating or preventing the disease or medical condition.

Other aspects of the invention will be apparent from the detailed description and claims that follow.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows AAV capsid protein insertion and substitution amino acid sequences encoding the amino acid sequences which were found in the non-human primate brain after two rounds of evaluation of engineered AAV libraries.

### DETAILED DESCRIPTION OF THE DISCLOSURE

In one aspect the disclosure provides rAAVs with high expression levels in the brain.

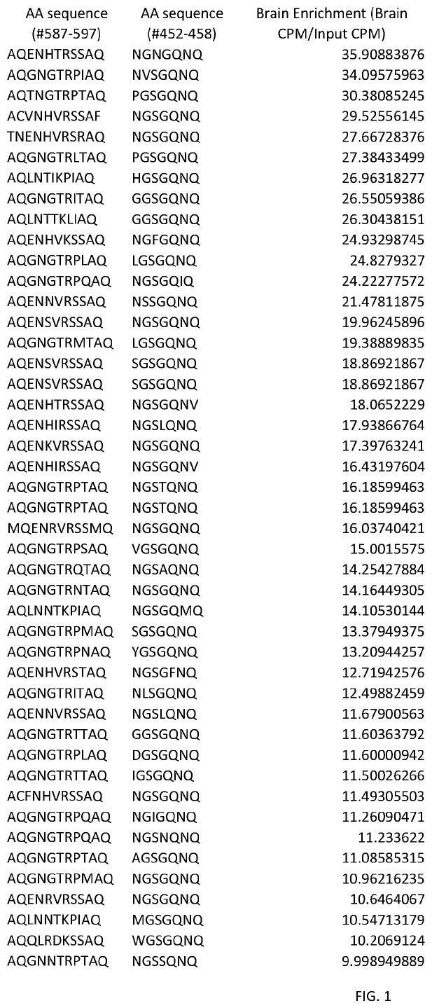

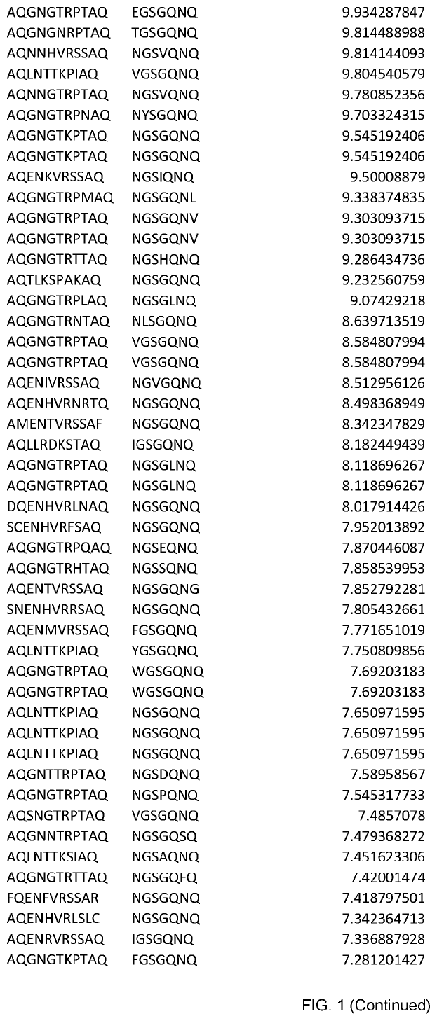

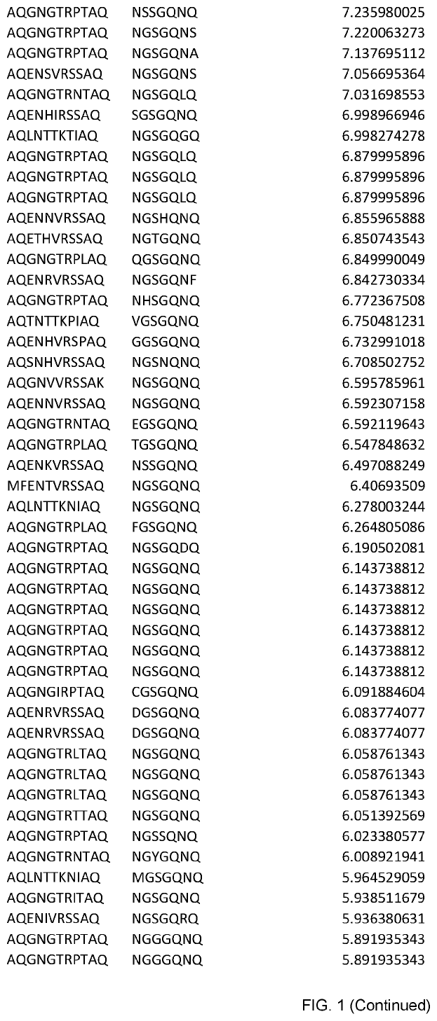

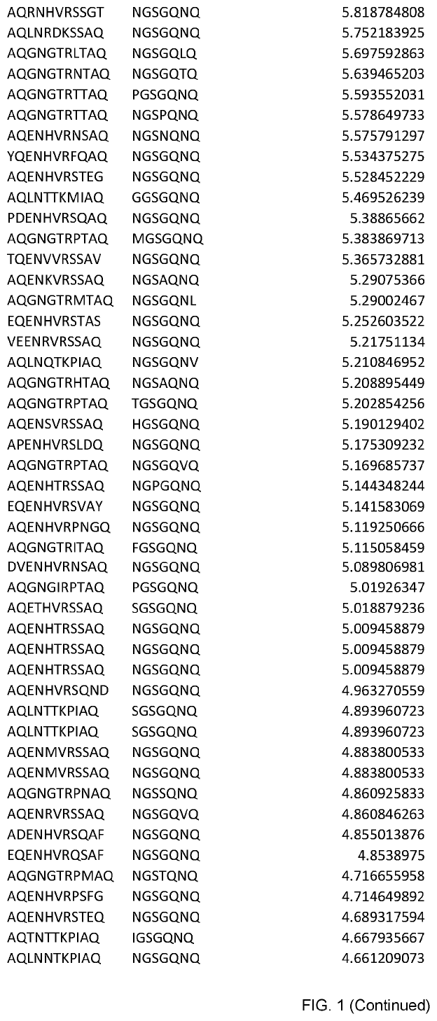

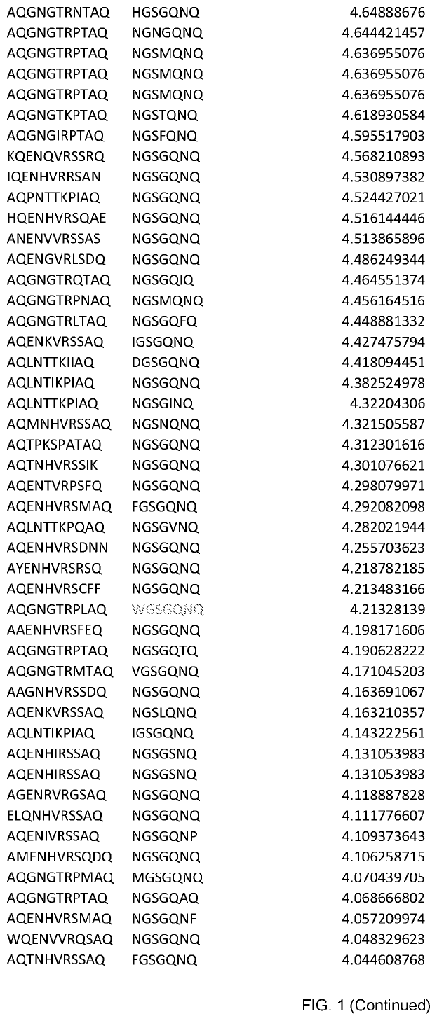

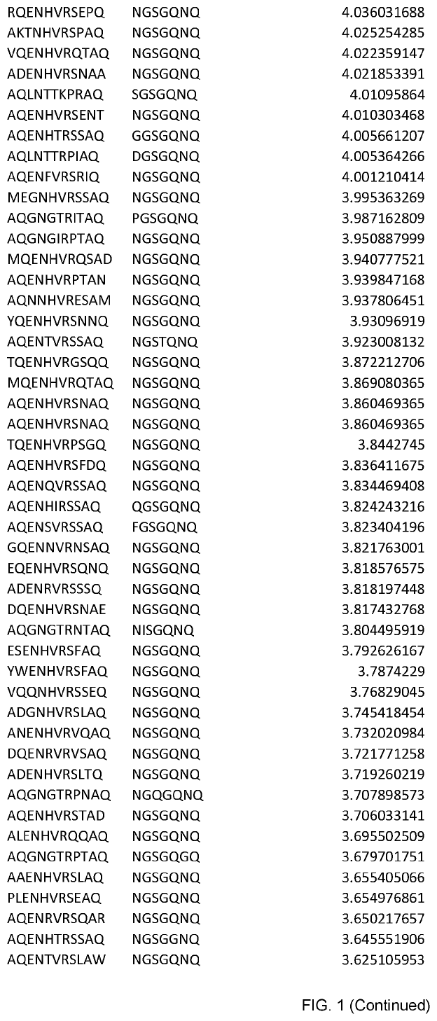

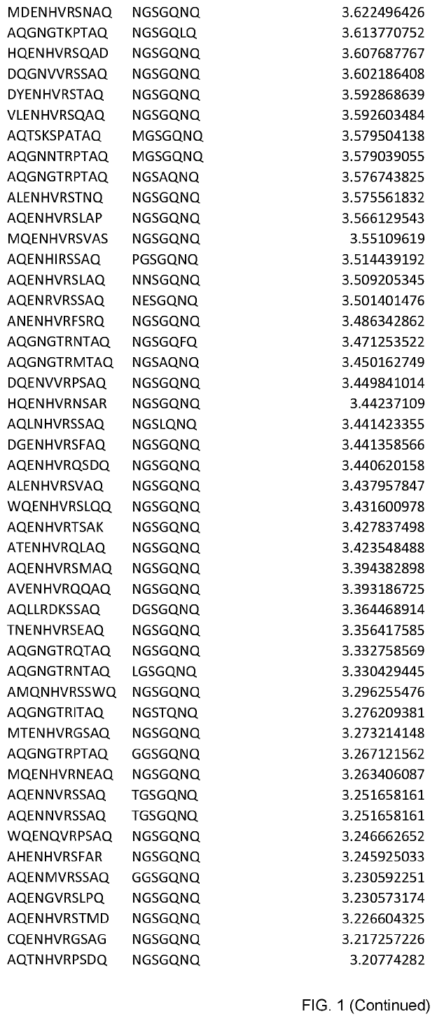

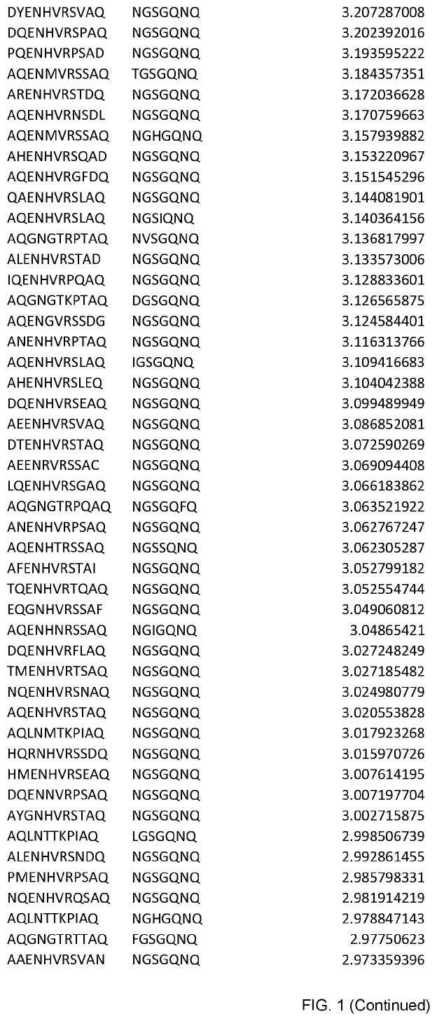

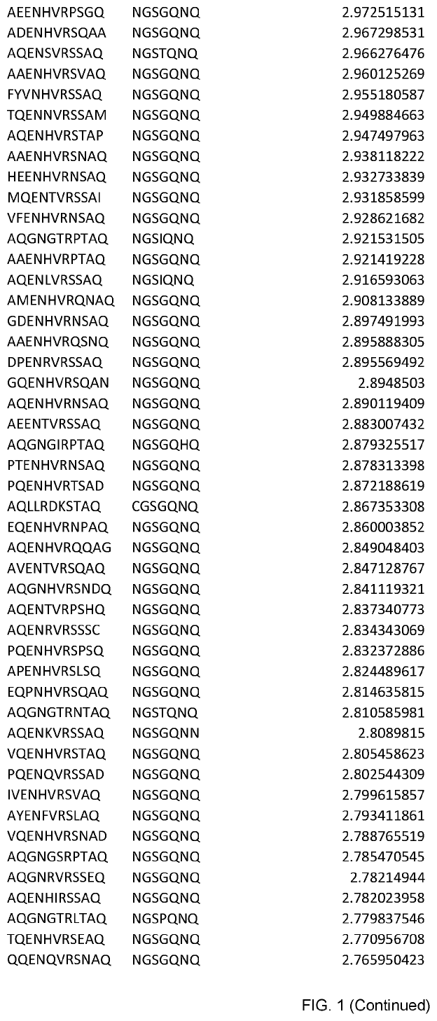

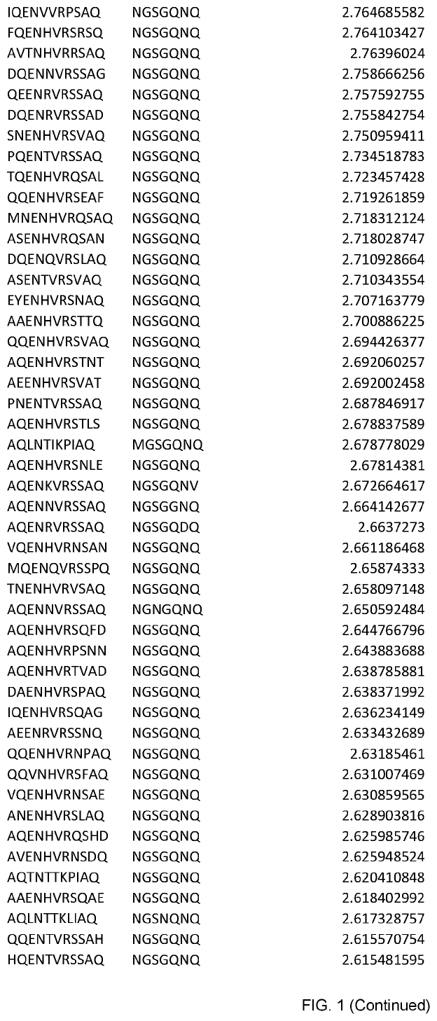

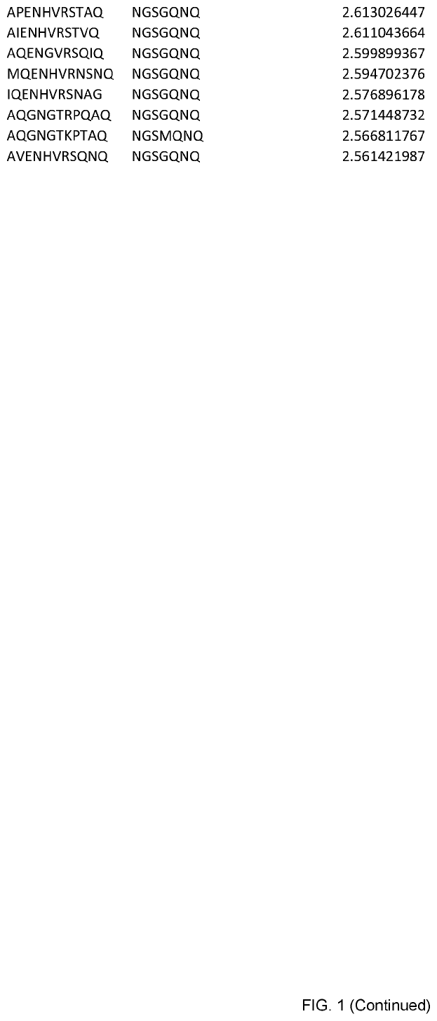

In one aspect, the disclosure provides rAAVs with a peptide insertion and or substitution comprising or consisting of an amino-acid sequence set forth in any one of Tables 1-3, FIG. 1 and and/or Formulas I-VI.

Aspects disclosed herein provide AAV capsids comprising an AAV capsid protein comprising an amino acid sequence of Formula I

E-N-H-V-X¹-X²-X³ (I)

wherein X¹ is an amino acid selected from K and R; wherein X² is an amino acid selected from F, G, L, N, P, Q, R, S and V; and X³ is an amino acid selected from C, D, E, F, L, M, N, P, Q, R, S, T and V.

In some embodiments, the AAV capsid protein comprises an amino acid sequence of formula I wherein X³ is S.

In some embodiments, the AAV capsid protein comprises an amino acid sequence of formula I wherein X³ is R.

In some embodiments, the AAV capsid protein comprises an amino acid sequence of formula I wherein X³ is T.

In some embodiments, the AAV capsid protein comprises an amino acid sequence of formula I wherein X² is S.

In some embodiments, the AAV capsid protein comprises an amino acid sequence of formula I wherein X² is N.

In some embodiments, the AAV capsid protein comprises an amino acid sequence of formula I wherein X² is R, F or L.

In some embodiments, the AAV capsid protein comprises an amino acid sequence of formula I wherein X¹ is R.

In some embodiments, the AAV capsid protein comprises an amino acid sequence of formula I wherein X¹ is K.

In some embodiments, the sequence as described in Formula I is selected from ENHVRSR, ENHVKSS, ENHVRST, ENHVRNR, ENHVRRS, ENHVRFS, and ENHVRLS.

Aspects disclosed herein provide AAV capsids comprising an AAV capsid protein comprising an amino acid sequence of Formula II

X⁴-X⁵-X⁶-X⁷-R-S-S (II)

wherein X⁴ is an amino acid selected from E, F, G, L, M, N, Q, R, S, T and V; X⁵ is an amino acid selected N and T; X⁶ is an amino acid selected F, H, I, K, M, N, Q, R, S, T and V; and X⁷ is an amino acid selected from I, N, T and V.

In some embodiments, the AAV capsid protein comprises an amino acid sequence of formula II wherein X⁵ is N.

In some embodiments, the AAV capsid protein comprises an amino acid sequence of formula II wherein X⁷ is V.

In some embodiments, the sequence as described in Formula II is selected from ENHTRSS, VNHVRSS, ENNVRSS, ENSVRSS, ENHTRSS, ENHIRSS, ENKVRSS, ENRVRSS, FNHVRSS, and NNHVRSS.

Aspects disclosed herein provide AAV capsids comprising an AAV capsid protein comprising an amino acid sequence of Formula III

X⁸-N-G-T-R-X⁹-X¹⁰ (III)

wherein X⁸ is an amino acid selected from G, N, S and T; X⁹ is an amino acid selected from H, I, L, M, N, P, Q and T; and X¹⁰ is an amino acid selected from I, L, M, N, Q, S and T.

In some embodiments, the AAV capsid protein comprises an amino acid sequence of formula III wherein X⁸ is G.

In some embodiments, the AAV capsid protein comprises an amino acid sequence of formula III wherein X⁸ is T.

In some embodiments, the AAV capsid protein comprises an amino acid sequence of formula III wherein X⁹ is P.

In some embodiments, the AAV capsid protein comprises an amino acid sequence of formula III wherein X⁹ is I or L.

In some embodiments, the AAV capsid protein comprises an amino acid sequence of formula III wherein X¹⁰ is I or T.

In some embodiments, the sequence as described in Formula III, is selected from GNGTRPI, TNGTRPT, GNGTRLT, GNGTRIT, GNGTRPL, and GNGTRPQ.

In some embodiments, the sequence as described in Table 1, is selected from GNGTRLT, ENHTRSS, GNGTRPI, TNGTRPT, VNHVRSS, LNTIKPI, GNGTRIT, LNTTKLI, ENHVKSS, GNGTRPL, GNGTRPQ, ENNVRSS, and ENSVRSS.

In some embodiments, the sequence as described in Table 1 is selected from AQGNGTRLTAQ, AQENHTRSSAQ, AQGNGTRPIAQ, AQTNGTRPTAQ, ACVNHVRSSAF, TNENHVRSRAQ, AQLNTIKPIAQ, AQGNGTRITAQ, AQLNTTKLIAQ, AQENHVKSSAQ, AQGNGTRPLAQ, AQGNGTRPQAQ, AQENNVRSSAQ, and AQENSVRSSAQ.

In some embodiments, the insertion sequence is represented by the peptide sequences listed in Table 1.

TABLE 1

| Sequence | Sequence |
|---|---|
| ENHTRSS | AQENHTRSSAQ |
| GNGTRPI | AQGNGTRPIAQ |
| TNGTRPT | AQTNGTRPTAQ |
| VNHVRSS | ACVNHVRSSAF |
| ENHVRSR | TNENHVRSRAQ |
| GNGTRLT | AQGNGTRLTAQ |
| LNTIKPI | AQLNTIKPIAQ |
| GNGTRIT | AQGNGTRITAQ |
| LNTTKLI | AQLNTTKLIAQ |
| ENHVKSS | AQENHVKSSAQ |
| GNGTRPL | AQGNGTRPLAQ |
| GNGTRPQ | AQGNGTRPQAQ |
| ENNVRSS | AQENNVRSSAQ |
| ENSVRSS | AQENSVRSSAQ |
| GNGTRMT | AQGNGTRMTAQ |

Aspects disclosed herein provide AAV capsids comprising an AAV capsid protein comprising an amino acid sequence of Formula IV at 452-458

X¹¹-X¹²-X¹³-X¹⁴-X¹⁵-N-Q (IV)

wherein X¹¹ is an amino acid selected from A, D, G, H, I, L, M, N, P, Q, S, V, W and Y; X¹² is an amino acid selected from G, L, N, P, S and V; X¹³ is an amino acid selected from F, I, N, and S; X¹⁴ is an amino acid selected from A, G, L, N and T; and X¹⁵ is an amino acid selected from F and Q.

In some embodiments, the AAV capsid protein comprises an amino acid sequence of formula IV wherein X¹¹ is an amino acid selected from A, G, H, L, N, P, Q, S, V and W.

In some embodiments, the AAV capsid protein comprises an amino acid sequence of formula IV wherein X¹² is G, N, P or V.

In some embodiments, the AAV capsid protein comprises an amino acid sequence of formula IV wherein X¹³ is F, N or S.

In some embodiments, the AAV capsid protein comprises an amino acid sequence of formula IV wherein X¹⁴ is G.

In some embodiments, the AAV capsid protein comprises an amino acid sequence of formula IV wherein X¹⁵ is Q.

In some embodiments, the sequence as described in Formula IV, is selected from AGSGQNQ, DGSGQNQ, GGSGQNQ, HGSGQNQ, IGSGQNQ, LGSGQNQ, MGSGQNQ, NGFGQNQ, NGIGQNQ, NGNGQNQ, NGSAQNQ, NGSGENQ, NGSGQIQ, NGSGQMQ, NGSGQNQ, NGSGQNV, NGSLQNQ, NGSNQNQ, NGSTQNQ, NLSGQNQ, NSSGQNQ, PGSGQNQ, SGSGQNQ, VGSGQNQ, WGSGQNQ, and YGSGQNQ.

In some embodiments, the sequence as described in Formula IV, is selected from AGSGQNQ, DGSGQNQ, GGSGQNQ, HGSGQNQ, IGSGQNQ, LGSGQNQ, MGSGQNQ, NGFGQNQ, NGIGQNQ, NGNGQNQ, NGSAQNQ, NGSGENQ, NGSGQIQ, NGSGQMQ, NGSGQNQ, NGSGQNV, NGSLQNQ, NGSNQNQ, NGSTQNQ, NLSGQNQ, NSSGQNQ, PGSGQNQ, SGSGQNQ, VGSGQNQ, WGSGQNQ, and YGSGQNQ.

In some embodiments, the sequence as described in Formula IV, comprising a sequence at AA 452-458 selected from DGSGQNQ, FGSGQNQ, GGSGQNQ, IGSGQNQ, MGSGQNQ, NGSAQNQ, NGSGQFG, NGSGQLQ, NGSGQNQ, NGSGQNV, NGSLQNQ, NGSMQNQ, NGSSQNQ, NGSTQNQ, PGSGQNQ, QGSGQNQ, SGSGQNQ, TGSGQNQ, VGSGQNQ, and WGSGQNQ.

In some embodiments, the sequence as described in Formula IV, comprising a sequence at AA 452-458 selected from NGSGQNQ, NGSMQNQ, and NGKSSNQ.

Aspects disclosed herein provide AAV capsids comprising an AAV capsid protein comprising an amino acid sequence of Formula V at 452-458

X¹⁶-G-S-G-Q-N-Q (V)

wherein X³² is an amino acid selected from A, D, G, H, I, L, M, N, P, S, W and Y.

Aspects disclosed herein provide AAV capsids comprising an AAV capsid protein comprising an amino acid sequence of Formula VI at 452-458

N-G-S-G-X¹⁷-X¹⁸-X¹⁹ (VI)

wherein X¹⁷ is an amino acid selected from F and Q; X¹⁸ is an amino acid selected from I, M and N; and X¹⁹ is an amino acid selected from Q and V.

In some embodiments, the 452-458 substitution sequence is represented by the peptide sequences listed in Table 2.

TABLE 2
Sequence

AGSGQNQ
APSGQNQ
GGSGQNQ
HGSGQNQ
LGSGQNQ
NGNGQNQ
NVSGQNQ
NGSGQNQ
NGFGQNQ
NGSGQIQ
NSSGQNQ
NGSPGIQ
PGSGQNQ
QGSGQPQ
SGSMQNN
SGSGQNQ
VNHGQNQ
WGSGQNQ

Generally, the insertion comprises a five-, six-, or seven-amino acid sequence (5-mer, 6-mer, or 7-mer, respectively) that is inserted or substituted at the 588 loop in a parental AAV capsid protein. Aspects provided herein provide amino acid insertions comprising seven amino acid polymer (7-mer) inserted at AA588-589, and may additionally include a substitution of one or two amino acids at amino acid positions flanking the 7-mer sequence (e.g., AA587-588 and/or AA589-590) to produce an eleven amino acid polymer (11-mer) at the 588 loop of a parental AAV capsid protein.

Aspects disclosed herein provide AAV capsids comprising an AAV capsid protein comprising substituting an amino acid sequence at amino acids 587-590 selected from AAAQ, AADQ, AAEQ, ACAF, ADAA, ADAF, ADAH, ADAL, ADAQ, ADRK, ADSQ, ADTQ, AEAC, AEAQ, AFAI, AGAQ, AHAD, AHAR, AHEQ, AKAQ, ALAD, ALAQ, ALNQ, AMDQ, AMGF, AMNQ, AMNY, AMWQ, ANAQ, ANAS, ANRQ, ANSD, APDQ, AQAD, AQAK, AQAM, AQAN, AQAP, AQAR, AQAW, AQCG, AQDD, AQDG, AQDL, AQDQ, AQEQ, AQFF, AQFG, AQFQ, AQGQ, AQIK, AQIQ, AQND, AQNN, AQNT, AQPG, AQWD, ARDQ, ARGK, ASWW, ATAQ, AVAQ, AYAQ, AYSQ, COAG, DGAQ, DNNQ, DNYQ, DQAE, DQAQ, DQLD, DONS, DTAQ, DVAQ, DYAQ, ELAG, ELAQ, EMGQ, EQAF, EQAS, EQAY, EQGQ, EQNQ, ERAE, ESAQ, GQAQ, GQAY, GQPM, HMAQ, HQAD, HQAE, HQAR, HQDQ, IDAN, IEVQ, IHPQ, IMEQ, INAP, IQAN, IQAQ, KNHQ, KQRQ, LETQ, LPAN, LQAQ, LTAP, MDAQ, MEAQ, MFAQ, MLAV, MLDQ, MQAD, MQAQ, MQAS, MQMQ, MQPD, MTAQ, MTPQ, NDAH, NDAS, NQAQ, PAAD, PDAQ, PLAQ, PQAD, PQAE, QAAQ, RQPQ, SDAN, TMAQ, TNAN, TNAQ, TQAQ, TQDG, TQGQ, TQQQ, VEAG, VEAQ, VLAQ, VQAQ, VODA, VQEQ, WQAQ, WQQQ, YQAQ, YQNQ, and YWAQ.

In some embodiments, the sequence AAV capsid protein comprising substituting an amino acid sequence at amino acids 587-590 selected from ACAF, TNAQ, AMAF, SCAQ, SNAQ, FQAR, AQLC, AQAK, AQGT, AQEG, AQGQ, APDQ, TQAV, EQAS, MFAQ, VEAQ, EQAY, DVAQ, and MQMQ.

In some aspects, the insertion amino acid sequence is at least 71.4% identical to the amino acid sequence provided in Tables 1-3, FIG. 1 and and/or Formulas I-VI. In some aspects, the insertion amino acid sequence is at least 86.7% identical to the amino acid sequence provided in Tables 1-3, FIG. 1 and and/or Formulas I-VI.

Also disclosed herein are methods and kits for producing therapeutic recombinant AAV (rAAV) particles, as well as methods and pharmaceutical compositions or formulations comprising the rAAV particles, for the treatment of a disease or condition affecting the brain.

Disclosed herein are AAV capsids engineered with increased viral transduction in the brain. The AAV capsids can encapsidate a viral vector with a heterologous nucleic acid encoding, for example, a therapeutic gene expression product. Transduction of the heterologous nucleic acid in the brain can be achieved upon systemic delivery to a subject of the AAV capsid of the present disclosure encapsidating a heterologous nucleic acid. The AAV capsids disclosed herein are advantageous for many applications of gene therapy to treat human disease, including, but not limited to, disorders of the central nervous system.

The recombinant AAV vectors comprising a nucleic acid sequence encoding the AAV capsid proteins of the present disclosure as also provided herein. For example, the viral vectors of the present disclosure comprise a nucleic acid sequence comprising the AAV viral Cap (Capsid) encoding VP1, VP2, and VP3, at least one of which is modified to produce the AAV capsid proteins of the present disclosure. The recombinant AAV vector provided can be derived from an AAV serotype (e.g., AAV9) or a variant AAV serotype including an insertion of the present invention.

### AAV Capsids

Provided herein are modified adeno-associated (AAV) virus capsid compositions useful for integrating a transgene into a target cell or environment (in a subject when they are administered systemically to the subject.

An rAAV comprises an AAV capsid that can be engineered to encapsidate a heterologous nucleic acid (e.g., therapeutic nucleic acid, gene editing machinery). The AAV capsid is made up of three AAV capsid protein monomers, VP1, VP2, and VP3. Sixty copies of these three VP proteins interact in a 1:1:10 ratio to form the viral capsid. VP1 covers the whole of VP2 protein in addition to a ˜137 amino acid N-terminal region (VP1u), VP2 covers the whole of VP3 in addition to ˜65 amino acid N-terminal region (VP1/2 common region). The three capsid proteins share a conserved amino acid sequence of VP3, which in some cases is the region beginning at amino acid position 138 (e.g., AA139-736).

While not wishing to be bound by theory, it is understood that a parent AAV capsid sequence comprises a VP1 region. In certain embodiments, a parent AAV capsid sequence comprises a VP1, VP2 and/or VP3 region, or any combination thereof. A parent VP1 sequence may be considered synonymous with a parent AAV capsid sequence.

The AAV VP3 structure contains highly conserved regions that are common to all serotypes, a core eight-stranded β-barrel motif (βB-βI) and a small α-helix (αA). The loop regions inserted between the β-strands consist of the distinctive HI loop between β-strands H and I, the DE loop between β-strands D and E, and nine variable regions (VRs), which form the top of the loops. These VRs, such as the AA588 loop, are found on the capsid surface and can be associated with specific functional roles in the AAV life cycle including receptor binding, transduction and antigenic specificity.

In some aspects, the rAAV variant of the present invention comprises an AAV capsid protein having a peptide insertion at the residues corresponding to amino acids 588-589 of the AAV9 native sequence of SEQ ID NO: 1.

The AAV capsids comprise AAV capsid proteins (e.g., VP1, VP2, and VP3), each with an insertion, such as in the 588 loop of a parental AAV capsid protein structure (AAV9 VP1 numbering). The 588 loop contains the site of heparan sulfate binding of AAV2 and is amenable to peptide display. The only known receptors for AAV9 is N-linked terminal galactose and AAV receptor (AAVR), but many indications point toward there being others. Modifications to AAV9 588 loop are shown herein to confer an increased transgene transduction in target in vivo environments.

The present invention provides, in an aspect, a peptide insertion at the AAV 588 loop comprising or consisting of an amino-acid sequence set forth in any one of Tables 1-3, FIG. 1 and and/or Formulas I-VI.

Disclosed herein are AAV capsids comprising AAV capsid proteins with an insertion at the 588 loop that confer a higher transduction in brain cell types (e.g., brain endothelial cells, neurons, astrocytes). In particular, the AAV capsid proteins disclosed herein enable rAAV-mediated transduction of a heterologous nucleic acid (e.g., transgene) in the brain of a subject. The AAV capsids of the present disclosure may be formulated as a pharmaceutical composition. In addition, the AAV capsids can be isolated and purified to be used for a variety of applications.

In some embodiments, the rAAV capsid of the present disclosure are generated using the methods disclosed herein. In some instances, the rAAV capsid is chimeric. In some instances, the rAAV, or variant AAV protein comprises therein, confer an increase in a localization of the rAAV within the target tissue, as compared to the parental AAV capsid or capsid protein.

### AAV Capsid Proteins

Disclosed herein are recombinant AAV (rAAV) capsids which comprise AAV capsid proteins that are engineered with a modified capsid protein (e.g., VP1, VP2, VP3). In some embodiments, the rAAV capsid proteins of the present disclosure are generated using the methods disclosed herein. In some embodiments, the AAV capsid proteins are used in the methods of delivering a therapeutic nucleic acid (e.g., a transgene) to a subject. In some instances, the rAAV capsid proteins have desired AAV expression rendering them particularly suitable for certain therapeutic applications, e.g., the treatment of a disease or disorder in a subject such as those disclosed herein.

The rAAV capsid proteins are engineered for optimized expression in the brain, for example the brain, of a subject upon systemic administration of the rAAV to the subject. The rAAV capsid proteins are engineered to include the insertions provided in Tables 1-3, FIG. 1 and and/or Formulas I-VI. The rAAV capsid proteins including the insertions provided in Tables 1-3, FIG. 1 and and/or Formulas I-VI are engineered to achieve efficient transduction of an encapsidated transgene. In particular, the rAAV capsid proteins have increased expression in the brain of a subject.

The engineered AAV capsid proteins described herein have, in some cases, an insertion of an amino acid that is heterologous to the parental AAV capsid protein at amino acid positions in the 588 loop. In some embodiments, the amino acid is not endogenous to the parental AAV capsid protein at the amino acid position of the insertion. The amino acid may be a naturally occurring amino acid in the same or equivalent amino acid position as the insertion of the substitution in a different AAV capsid protein.

The 7-mers described herein were advantageously generated using polymerase chain reaction (PCR) with degenerate primers, where each of the seven amino acids is encoded by a deoxyribose nucleic acid (DNA) sequence N-N-K. “N” is any of the four DNA nucleotides and K is guanine (G) or thymine (T). This method of generating random 7-mer amino acid sequences enables 1.28 billion possible combinations at the protein level.

The rAAV capsid proteins of the present disclosure comprise an insertion of an amino acid in an amino acid sequence of an AAV capsid protein. The AAV capsid, from which an engineered AAV capsid protein of the present disclosure is produced, is referred to as a “parental” AAV capsid. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562.

In some cases, the parental AAV is derived from an AAV with a serotype selected from AAV1, AAV2, AAV3, AAV4, AAV5, AAV6, AAV7, AAV8, AAV9, AAV10, AAV11 and AAV12. The AAV capsid protein that is “derived” from another may be a variant AAV capsid protein. A variant may include, for example, a heterologous amino acid in an amino acid sequence of the AAV capsid protein. The heterologous amino acid may be non-naturally occurring in the AAV capsid protein. The heterologous amino acid may be naturally occurring in a different AAV capsid protein. In some instances, the parental AAV capsid is described in US Pat Publication 2020/0165576 and U.S. Pat. App. Ser. No. 62/832,826 and PCT/US20/20778; the content of each of which is incorporated herein.

In some instances, the parental AAV is AAV9. In some instances, the amino acid sequence of the AAV9 capsid protein comprises SEQ ID NO: 1. The amino acid sequence of AAV9 VP1 capsid protein (>tr|Q6JC40|Q6JC40_9VIRU Capsid protein VP1 OS=Adeno-associated virus 9 OX=235455 GN=cap PE=1 SV=1) is provided in SEQ ID NO: 1 (MAADGYLPDWLEDNLSEGIREWWALKPGAPQPKANQQHQDNARGLVLPGYKYLGPG NGLDKGEPVNAADAAALEHDKAYDQQLKAGDNPYLKYNHADAEFQERLKEDTSFGG NLGRAVFQAKKRLLEPLGLVEEAAKTAPGKKRPVEQSPQEPDSSAGIGKSGAQPAKKRL NFGQTGDTESVPDPQPIGEPPAAPSGVGSLTMASGGGAPVADNNEGADGVGSSSGNWH CDSQWLGDR VITTSTRTWALPTYNNHLYKQISNSTSGGSSNDNAYFGYSTPWGYFDFN RFHCHFSPRDWQRLINNNWGFRPKRLNFKLFNIQVKEVTDNNGVKTIANNLTSTVQVFT DSDYQLPYVLGSAHEGCLPPFPADVFMIPQYGYLTLNDGSQAVGRSSFYCLEYFPSQML RTGNNFQFSYEFENVPFHSSYAHSQSLDRLMNPLIDQYLYYLSKTINGSGQNQQTLKFSV AGPSNMAVQGRNYIPGPSYRQQRVSTTVTQNNNSEFAWPGASSWALNGRNSLMNPGP AMASHKEGEDRFFPLSGSLIFGKQGTGRDNVDADKVMITNEEEIKTTNPVATESYGQVA TNHQSAQAQAQTGWVQNQGILPGMVWQDRDVYLQGPIWAKIPHTDGNFHPSPLMGGF GMKHPPPQILIKNTPVPADPPTAFNKDKLNSFITQYSTGQVSVEIEWELQKENSKRWNPE IQYTSNYYKSNNVEFAVNTEGVYSEPRPIGTRYLTRNL). In some instances, the parental AAV capsid protein sequence is 70%, 75%, 80%, 85%, 90%, 91%, 92%, 93%, 94%, 95%, 96%, 97%, 98%, 99%, or 100% homologous to SEQ ID NO: 1.

AAV capsid proteins from native AAV serotypes, such as AAV9, with tropisms including the liver activate the innate immune response, which in some cases causes a severe inflammatory response in a subject, which can lead to multi-organ failure. By improving transduction of a native AAV serotype for a target in vivo tissue (e.g., brain), the rAAV particles of the present disclosure reduce the immunogenic properties of AAV-mediated transgene delivery and prevent activation of the innate immune response.

In some instances, the parental AAV capsid protein comprises the entire VP1 region provided in SEQ ID NO: 1 (e.g., amino acids 1-736). In some instances, the parental AAV capsid protein comprises amino acids 217-736 in SEQ ID NO: 1, which is the common region found in VP1, VP2 and VP3 AAV9 capsid proteins. In some instances, the AAV capsid protein comprises amino acids 64-736 in SEQ ID NO: 1, which is the common region found in VP1 and VP2. The parental AAV capsid protein sequence may comprise amino acids selected from 1-736, 10-736, 20-736, 30-736, 40-736, 50-736, 60-736, 70-736, 80-736, 90-736, 100-736, 110-736, 120-736, 130-736, 140-736, 150-736, 160-736, 170-736, 180-736, 190-736, 200-736, 210-736, 220-736, 230-736, 240-736, 250-736, 260-736, 270-736, 280-736, 290-736, 300-736, 310-736, 320-736, 330-736, 340-736, 350-736, 360-736, 370-736, 380-736, 390-736, 400-736, 410-736, 420-736, 430-736, 440-736, and 450-736, from SEQ ID NO: 1. In some aspects, the rAAV variant comprises an AAV capsid protein comprising an amino acid sequence that is at least 98% identical to amino acid 217 to amino acid 736 of SEQ ID NO: 1. In some instances, the amino acid insertion is at a three (3)-fold axis of symmetry of a corresponding parental AAV capsid protein.

Disclosed herein are insertions of an amino acid sequence in an AAV capsid protein. Where the sequence numbering designation “588-589” is noted for AAV9, for example AAV VP1, the invention also includes insertions in similar locations in the other AAV serotypes. As used herein, “AA588-589” indicates that the insertion of the amino acid (or amino acid sequence) is immediately after an amino acid (AA) at position 588 and immediately before an AA at position 589 within an amino acid sequence of a parental AAV VP capsid protein (VP1 numbering). Amino acids 587-591 include a motif comprising “AQAQA” as set forth in SEQ ID NO: 1. Exemplary AAV capsid protein sequences are provided in Table 3. For example, ENHTRSS is inserted at AA588-589 in an AAV9 capsid amino acid sequence, and provides variant A. It is envisioned that the sequences disclosed herein (Tables 1-3, FIG. 1 and Formulas I-VI) may be inserted at AA588-589 in an amino acid sequence of a parental AAV9 capsid protein or at AA587-590 (replacing amino acids AA587-590), a variant thereof, or equivalent amino acid position of a parental AAV of a different serotype (e.g., AAV1, AAV2, AAV3, and the like). In certain embodiments, the aforementioned “AQAQ” sequence flanking the insertion may include one or more substitutions. In any AAV capsid protein sequence disclosed herein, the amino acid at position 449 may be R or K.

TABLE 3
Exemplary AAV Capsid Protein Sequences

| Identifier | Sequence |
|---|---|
| Variant A | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTS [symbol] |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV |
| | FTDSDYQLPYVLGSAHEGCLPPFPADVFMIPQYGYLT [symbol] |
| | DGSQAVGRSSFYCLEYFPSQMLRTGNNFQFSYEFENV [symbol] |
| | HSSYAHSQSLDRLMNPLIDQYLYYLSRTINGNGQNQQ |
| | KFSVAGPSNMAVQGRNYIPGPSYRQQRVSTTVTQNNN |
| | FAWPGASSWALNGRNSLMNPGPAMASHKEGEDRFFP |
| | GSLIFGKQGTGRDNVDADKVMITNEEEIKTTNPVATES |
| | GQVATNHQSAQENHTRSSAQAQTGWVQNQGILPGM |
| | WQDRDVYLQGPIWAKIPHTDGNFHPSPLMGGFGMKH |
| | PQILIKNTPVPADPPTAFNKDKLNSFITQYSTGQVSVEI [symbol] |
| | ELQKENSKRWNPEIQYTSNYYKSNNVEFAVNTEGVYS |
| | RPIGTRYLTRNL |
| Variant B | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTS [symbol] |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV |
| | FTDSDYQLPYVLGSAHEGCLPPFPADVFMIPQYGYLT [symbol] |
| | DGSQAVGRSSFYCLEYFPSQMLRTGNNFQFSYEFENV [symbol] |
| | HSSYAHSQSLDRLMNPLIDQYLYYLSRTINVSGQNQQ |
| | KFSVAGPSNMAVQGRNYIPGPSYRQQRVSTTVTQNNN |
| | FAWPGASSWALNGRNSLMNPGPAMASHKEGEDRFFP |
| | GSLIFGKQGTGRDNVDADKVMITNEEEIKTTNPVATES |
| | GQVATNHQSAQGNGTRPIAQAQTGWVQNQGILPGM |
| | WQDRDVYLQGPIWAKIPHTDGNFHPSPLMGGFGMKH |
| | PQILIKNTPVPADPPTAFNKDKLNSFITQYSTGQVSVEI [symbol] |
| | ELQKENSKRWNPEIQYTSNYYKSNNVEFAVNTEGVYS |
| | RPIGTRYLTRNL |
| Variant C | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTS [symbol] |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV |
| | FTDSDYQLPYVLGSAHEGCLPPFPADVFMIPQYGYLTI |
| | DGSQAVGRSSFYCLEYFPSQMLRTGNNFQFSYEFENV [symbol] |
| | HSSYAHSQSLDRLMNPLIDQYLYYLSRTIPGSGQNQQ |
| | KFSVAGPSNMAVQGRNYIPGPSYRQQRVSTTVTQNNN |
| | FAWPGASSWALNGRNSLMNPGPAMASHKEGEDRFFP |
| | GSLIFGKQGTGRDNVDADKVMITNEEEIKTTNPVATES |
| | GQVATNHQSAQTNGTRPTAQAQTGWVQNQGILPGM |
| | WQDRDVYLQGPIWAKIPHTDGNFHPSPLMGGFGMKH |
| | PQILIKNTPVPADPPTAFNKDKLNSFITQYSTGQVSVEI [symbol] |
| | ELQKENSKRWNPEIQYTSNYYKSNNVEFAVNTEGVYS |
| | RPIGTRYLTRNL |
| Variant D | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTS [symbol] |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV [symbol] |
| | FTDSDYQLPYVLGSAHEGCLPPFPADVFMIPQYGYLT [symbol] |
| | DGSQAVGRSSFYCLEYFPSQMLRTGNNFQFSYEFENV [symbol] |
| | HSSYAHSQSLDRLMNPLIDQYLYYLSRTINGSGQNQQ [symbol] |
| | KFSVAGPSNMAVQGRNYIPGPSYRQQRVSTTVTQNN [symbol] |
| | FAWPGASSWALNGRNSLMNPGPAMASHKEGEDRFFP [symbol] |
| | GSLIFGKQGTGRDNVDADKVMITNEEEIKTTNPVATES |
| | GQVATNHQSACVNHVRSSAFAQTGWVQNQGILPGM [symbol] |
| | QDRDVYLQGPIWAKIPHTDGNFHPSPLMGGFGMKHP [symbol] |
| | ILIKNTPVPADPPTAFNKDKLNSFITQYSTGQVSVEIEW |
| | QKENSKRWNPEIQYTSNYYKSNNVEFAVNTEGVYSE [symbol] |
| | IGTRYLTRNL |
| Variant E | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTS [symbol] |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV |
| | FTDSDYQLPYVLGSAHEGCLPPFPADVFMIPQYGYLT [symbol] |
| | DGSQAVGRSSFYCLEYFPSQMLRTGNNFQFSYEFENV [symbol] |
| | HSSYAHSQSLDRLMNPLIDQYLYYLSRTINGSGQNQQ [symbol] |
| | KFSVAGPSNMAVQGRNYIPGPSYRQQRVSTTVTQNN [symbol] |
| | FAWPGASSWALNGRNSLMNPGPAMASHKEGEDRFFP |
| | GSLIFGKQGTGRDNVDADKVMITNEEEIKTTNPVATES |
| | GQVATNHQSTNENHVRSRAQAQTGWVQNQGILPGM |
| | WQDRDVYLQGPIWAKIPHTDGNFHPSPLMGGFGMKH |
| | PQILIKNTPVPADPPTAFNKDKLNSFITQYSTGQVSVEI [symbol] |
| | ELQKENSKRWNPEIQYTSNYYKSNNVEFAVNTEGVYS |
| | RPIGTRYLTRNL |
| Variant F | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTS [symbol] |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV |
| | FTDSDYQLPYVLGSAHEGCLPPFPADVFMIPQYGYLT [symbol] |
| | DGSQAVGRSSFYCLEYFPSQMLRTGNNFQFSYEFENV [symbol] |
| | HSSYAHSQSLDRLMNPLIDQYLYYLSRTIPGSGQNQQ |
| | KFSVAGPSNMAVQGRNYIPGPSYRQQRVSTTVTQNNN |
| | FAWPGASSWALNGRNSLMNPGPAMASHKEGEDRFFP |
| | GSLIFGKQGTGRDNVDADKVMITNEEEIKTTNPVATES |
| | GQVATNHQSAQGNGTRLTAQAQTGWVQNQGILPGM |
| | WQDRDVYLQGPIWAKIPHTDGNFHPSPLMGGFGMKH |
| | PQILIKNTPVPADPPTAFNKDKLNSFITQYSTGQVSVEI [symbol] |
| | ELQKENSKRWNPEIQYTSNYYKSNNVEFAVNTEGVYS |
| | RPIGTRYLTRNL |
| Variant G | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTSF |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV |
| | FTDSDYQLPYVLGSAHEGCLPPFPADVFMIPQYGYLT [symbol] |
| | DGSQAVGRSSFYCLEYFPSQMLRTGNNFQFSYEFENV [symbol] |
| | HSSYAHSQSLDRLMNPLIDQYLYYLSRTIHGSGQNQQ |
| | KFSVAGPSNMAVQGRNYIPGPSYRQQRVSTTVTQNNN |
| | FAWPGASSWALNGRNSLMNPGPAMASHKEGEDRFFP |
| | GSLIFGKQGTGRDNVDADKVMITNEEEIKTTNPVATES |
| | GQVATNHQSAQLNTIKPIAQAQTGWVQNQGILPGM [symbol] |
| | QDRDVYLQGPIWAKIPHTDGNFHPSPLMGGFGMKHP [symbol] |
| | ILIKNTPVPADPPTAFNKDKLNSFITQYSTGQVSVEIEW |
| | QKENSKRWNPEIQYTSNYYKSNNVEFAVNTEGVYSE [symbol] |
| | IGTRYLTRNL |
| Variant H | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTS [symbol] |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV |
| | FTDSDYQLPYVLGSAHEGCLPPFPADVFMIPQYGYLT [symbol] |
| | DGSQAVGRSSFYCLEYFPSQMLRTGNNFQFSYEFENV [symbol] |
| | HSSYAHSQSLDRLMNPLIDQYLYYLSRTIGGSGQNQQ |
| | KFSVAGPSNMAVQGRNYIPGPSYRQQRVSTTVTQNNN |
| | FAWPGASSWALNGRNSLMNPGPAMASHKEGEDRFFP |
| | GSLIFGKQGTGRDNVDADKVMITNEEEIKTTNPVATES |
| | GQVATNHQSAQGNGTRITAQAQTGWVQNQGILPGM |
| | WQDRDVYLQGPIWAKIPHTDGNFHPSPLMGGFGMKH |
| | PQILIKNTPVPADPPTAFNKDKLNSFITQYSTGQVSVEI [symbol] |
| | ELQKENSKRWNPEIQYTSNYYKSNNVEFAVNTEGVYS |
| | RPIGTRYLTRNL |
| Variant I | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTS [symbol] |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV |
| | FTDSDYQLPYVLGSAHEGCLPPFPADVFMIPQYGYLT [symbol] |
| | DGSQAVGRSSFYCLEYFPSQMLRTGNNFQFSYEFENV [symbol] |
| | HSSYAHSQSLDRLMNPLIDQYLYYLSRTIGGSGQNQQ |
| | KFSVAGPSNMAVQGRNYIPGPSYRQQRVSTTVTQNNN |
| | FAWPGASSWALNGRNSLMNPGPAMASHKEGEDRFFP |
| | GSLIFGKQGTGRDNVDADKVMITNEEEIKTTNPVATES |
| | GQVATNHQSAQLNTTKLIAQAQTGWVQNQGILPGM [symbol] |
| | QDRDVYLQGPIWAKIPHTDGNFHPSPLMGGFGMKHP [symbol] |
| | ILIKNTPVPADPPTAFNKDKLNSFITQYSTGQVSVEIEW |
| | QKENSKRWNPEIQYTSNYYKSNNVEFAVNTEGVYSE [symbol] |
| | IGTRYLTRNL |
| Variant J | MAADGYLPDWLEDNLSEGIREWWALKPGAPQPKAN [symbol] |
| | HQDNARGLVLPGYKYLGPGNGLDKGEPVNAADAAA [symbol] |
| | HDKAYDQQLKAGDNPYLKYNHADAEFQERLKEDTS [symbol] |
| | GNLGRAVFQAKKRLLEPLGLVEEAAKTAPGKKRPVE [symbol] |
| | QEPDSSAGIGKSGAQPAKKRLNFGQTGDTESVPDPQP [symbol] |
| | PPAAPSGVGSLTMASGGGAPVADNNEGADGVGSSSG [symbol] |
| | HCDSQWLGDRVITTSTRTWALPTYNNHLYKQISNSTS [symbol] |
| | SSNDNAYFGYSTPWGYFDFNRFHCHFSPRDWQRLINN |
| | WGFRPKRLNFKLFNIQVKEVTDNNGVKTIANNLTSTV |
| | FTDSDYQLPYVLGSAHEGCLPPFPADVFMIPQYGYLT [symbol] |
| | DGSQAVGRSSFYCLEYFPSQMLRTGNNFQFSYEFENV [symbol] |
| | HSSYAHSQSLDRLMNPLIDQYLYYLSRTINGFGQNQQ |
| | KFSVAGPSNMAVQGRNYIPGPSYRQQRVSTTVTQNNN |
| | FAWPGASSWALNGRNSLMNPGPAMASHKEGEDRFFP |
| | GSLIFGKQGTGRDNVDADKVMITNEEEIKTTNPVATES |
| | GQVATNHQSAQENHVKSSAQAQTGWVQNQGILPGM |
| | WQDRDVYLQGPIWAKIPHTDGNFHPSPLMGGFGMKH |
| | PQILIKNTPVPADPPTAFNKDKLNSFITQYSTGQVSVEI [symbol] |
| | ELQKENSKRWNPEIQYTSNYYKSNNVEFAVNTEGVYS |
| | RPIGTRYLTRNL |

[symbol]

indicates data missing or illegible when filed

The insertions described herein may, in some cases, comprise a 7-mer insertion at AA588-589. It is envisioned that any 7-mer insertion disclosed herein in addition to a substitution with any amino acid at amino acid positions 587-590 [AQAQ] may comprise an 11-mer.

Disclosed herein are AAV capsid proteins with an insertion described above in a parental AAV capsid protein that confers an increased transduction in the brain in a subject, even when delivered systemically. One of the many advantages of the AAV capsid proteins described herein is their ability to target tissue and cells within the brain. The tissue can be the brain. Non-limiting examples of brain cells include a neuron and a glial cell. Glial cells can be selected from an oligodendrocyte, an ependymal cell, an astrocyte and a microglia.

In some instances, the AAV capsid protein comprises an insertion of at least or about seven, eight, nine, ten or eleven amino acids of an amino acid sequence of Tables 1-3, FIG. 1 and Formulas I-VI at an amino acid position 588-589 or at 587-590 in a parental AAV9 capsid protein (SEQ ID NO: 1). In some cases, the AAV capsid protein has an increased viral transduction enrichment in brain.

The rAAV capsid proteins of the present disclosure may also have a substitution of an amino acid sequence at amino acid position 452-458 in a parental AAV9 capsid protein, or variant thereof, as described in WO2020068990. In some embodiments, the substitution of the amino acid sequence comprises NGNGQNQ at amino acid position 452-458 in the parental AAV9 capsid protein. In some embodiments, the substitution of the amino acid sequence comprises NGSGQNQ at amino acid position 452-458 in the parental AAV9 capsid protein.

The rAAV capsid proteins described herein may be isolated and purified. The AAV may be isolated and purified by methods standard in the art such as by column chromatography, iodixanol gradients, or cesium chloride gradients. Methods for purifying AAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

In addition, the AAV capsid proteins disclosed herein, either isolated and purified, or not, may be formulated into a pharmaceutical formulation, which in some cases, further comprises a pharmaceutically acceptable carrier.

The rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein.

“Percent Identity” is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see: Henikoff and Henikoff, (1989) Proc. Natl. Acad. Sci. USA 89: 10915).

Sequence identity/similarity values provided herein can refer to the value obtained using the BLAST+ 2.5.0 suite of programs using default settings (blast.ncbi.nlm.nih.gov) (Camacho, C., et al. (2009) BLAST+: architecture and applications. BMC Bioinformatics 10:421).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17: 149-63) and XNU (Ci-ayerie and States (1993) Comput. Chem. 17: 191-201) low-complexity filters can be employed alone or in combination.

The terms “substantial identity” and “substantially identical” indicate that a polypeptide or nucleic acid comprises a sequence with between 55-100% sequence identity to a reference sequence, with at least 55% sequence identity, or at least 60%, or at least 65%, or at least 70%, or at least 75%, or at least 80%, or at least 85%, or at least 90%, or at least 95%, or at least 99% sequence identity or any percentage of value within the range of 55-100% sequence identity relative to the reference sequence. The percent sequence identity may occur over a specified comparison window. Optimal alignment may be ascertained or conducted using the homology alignment algorithm of Needleman and Wunsch, supra.

For example, the insertion sequences may include, but are not limited to, sequences that are not exactly the same as the sequences disclosed herein, but which have, in addition to the substitutions explicitly described for various sequences listed herein, additional substitutions of amino acid residues which substantially do not impair the activity or properties of the sequences described herein, such as those predicted by homology software e.g. BLOSUM62 matrices.

### AAV Particles

The rAAV particles with the insertion sequences described herein have an increased transduction enrichment in the brain. In some instances, the increased transduction enrichment comprises a 1-fold, 2-fold, 3-fold, 4-fold, 5-fold, 6-fold, 7-fold, 8-fold, 9-fold or 10-fold increase, or more. In some instances, the increased transduction enrichment is at least 1-fold. In some instances, the increased transduction enrichment is at least 2-fold. In some instances, the increased transduction enrichment is at least 4-fold.

The rAAV particles with the insertion sequences described herein have an increased expression enrichment in the brain. Detecting whether a rAAV possesses more or less specificity for a target in vivo environment, includes measuring a level of gene expression product (e.g., RNA or protein) expressed from the heterologous nucleic acid encapsidated by the rAAV in a tissue sample obtained from a subject. Suitable methods for measuring expression of a gene expression product include next-generation sequencing (NGS) and quantitative polymerase chain reaction (qPCR).

The increased expression in the brain is represented by the enrichment values provided in FIG. 1.

### Heterologous Nucleic Acids

Disclosed herein are therapeutic nucleic acids useful for the treatment or prevention of a disease or condition, or symptom of the disease or condition. In some embodiments, the therapeutic nucleic acids encode a therapeutic gene expression product. Non-limiting examples of gene expression products include proteins, polypeptides, peptides, enzymes, antibodies, antigen binding fragments, nucleic acid (RNA, DNA, antisense oligonucleotide, siRNA, and the like), and gene editing components, for use in the treatment, prophylaxis, and/or amelioration of the disease or disorder, or symptoms of the disease or disorder. In some instances, the therapeutic nucleic acids are placed in an organism, cell, tissue or organ of a subject by way of a rAAV, such as those disclosed herein.

Disclosed herein are rAAVs, each comprising a viral vector (e.g., a single stranded DNA molecule (ssDNA)). In some instances, the viral vector comprises two inverted terminal repeat (ITR) sequences that are about 145 bases each, flanking a transgene. In some embodiments, the transgene comprises a therapeutic nucleic acid, and in some cases, a promoter in cis with the therapeutic nucleic acid in an open reading frame (ORF). The promoter is capable of initiating transcription of therapeutic nucleic acid in the nucleus of the target cell. The ITR sequences can be from any AAV serotype. Non-limiting examples of AAV serotypes include AAV1, AAV2, AAV3, AAV4, AAV5, AAV6, AAV7, AAV8, AAV9, AAV10, AAV11, and AAV12. In some cases, an ITR is from AAV2. In some cases, an ITR is from AAV9.

Disclosed herein are transgenes that can comprise any number of nucleotides. In some cases, a transgene can comprise less than about 100 nucleotides. In some cases, a transgene can comprise at least about 100 nucleotides. In some cases, a transgene can comprise at least about 200 nucleotides. In some cases, a transgene can comprise at least about 300 nucleotides. In some cases, a transgene can comprise at least about 400 nucleotides. In some cases, a transgene can comprise at least about 500 nucleotides. In some cases, a transgene can comprise at least about 1000 nucleotides. In some cases, a transgene can comprise at least about 5000 nucleotides. In some cases, a transgene can comprise over 5,000 nucleotides. In some cases, a transgene can comprise between about 500 and about 5000 nucleotides. In some cases, a transgene comprises about 5000 nucleotides. In any of the cases disclosed herein, the transgene can comprise DNA, RNA, or a hybrid of DNA and RNA. In some cases, the transgene can be single stranded. In some cases, the transgene can be double stranded.

Disclosed herein are transgenes useful for modulating the expression or activity of a target gene or gene expression product thereof. In some instances, the transgene is encapsidated by an rAAV capsid protein of an rAAV particle described herein. In some instances, the rAAV particle is delivered to a subject to treat a disease or condition disclosed herein in the subject. In some instances, the delivery is systemic.

The transgenes disclosed herein are useful for expressing an endogenous gene at a level similar to that of a healthy or normal individual. This is particularly useful in the treatment of a disease or condition related to the under-expression, or lack of expression, of a gene expression product. In some embodiments, the transgenes disclosed herein are useful for overexpressing an endogenous gene, such that an expression level of the endogenous gene is above the expression level of a healthy or normal individual. Additionally, transgenes can be used to express exogenous genes (e.g., active agent such as an antibody, peptide, nucleic acid, or gene editing components). In some embodiments, the therapeutic gene expression product is capable of altering, enhancing, increasing, or inducing the activity of one or more endogenous biological processes in the cell. In some embodiments, the transgenes disclosed herein are useful for reducing expression of an endogenous gene, for example, a dominant negative gene. In some embodiments, the therapeutic gene expression product is capable of altering, inhibiting, reducing, preventing, eliminating, or impairing the activity of one or more endogenous biological processes in the cell. In some aspects, the increase of gene expression refers to an increase by at least about 20%, 30%, 40%, 50%, 60%, 70%, 80%, 85%, 90%, 95% and 100%. In one aspect, the protein product of the targeted gene may be increased by at least about 20%, 30%, 40%, 50%, 60%, 70%, 80%, 85%, 90%, 95% and 100%. In some aspects, the decrease of gene expression refers to an increase by at least about 20%, 30%, 40%, 50%, 60%, 70%, 80%, 85%, 90%, 95% and 100%. In one aspect, the protein product of the targeted gene may be decreased by at least about 20%, 30%, 40%, 50%, 60%, 70%, 80%, 85%, 90%, 95% and 100%.

When endogenous sequences (endogenous or part of a transgene) are expressed with a transgene, the endogenous sequences can be full-length sequences (wild-type or mutant) or partial sequences. The endogenous sequences can be functional. Non-limiting examples of the function of these full length or partial sequences include increasing the serum half-life of the polypeptide expressed by a transgene (e.g., therapeutic gene) and/or acting as a carrier.

A transgene can be inserted into an endogenous gene such that all, some or none of the endogenous gene is expressed. For example, a transgene as described herein can be inserted into an endogenous locus such that some (N-terminal and/or C-terminal to a transgene) or none of the endogenous sequences are expressed, for example as a fusion with a transgene. In other cases, a transgene (e.g., with or without additional coding sequences of the endogenous gene) is integrated into any endogenous locus, for example a safe-harbor locus. For example, a Frataxin (FXN) transgene can be inserted into an endogenous FXN gene. A transgene can be inserted into any gene, e.g., the genes as described herein.

At least one advantage of the present disclosure is that virtually any therapeutic nucleic acid may be used to express any therapeutic gene expression product. In some instances, the therapeutic gene expression product is a therapeutic protein or a peptide (e.g., antibody, antigen-binding fragment, peptide, or protein). In one embodiment the protein encoded by the therapeutic nucleic acid is between 50-5000 amino acids in length. In some embodiments the protein encoded is between 50-2000 amino acids in length. In some embodiments the protein encoded is between 50-1000 amino acids in length. In some embodiments the protein encoded is between 50-1500 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length. In some embodiments the peptide encoded is between 4-50 amino acids in length. In some embodiments, the protein encoded is a tetrapeptide, a pentapeptide, a hexapeptide, a heptapeptide, an octapeptide, a nonapeptide, or a decapeptide. In some embodiments, the protein encoded comprises a peptide of 2-30 amino acids, such as for example 5-30, 10-30, 2-25, 5-25, 10-25, or 10-20 amino acids. In some embodiments, the protein encoded comprises a peptide of at least 11, 12, 13, 14, 15, 17, 20, 25 or 30 amino acids, or a peptide that is no longer than 50 amino acids, e.g. no longer than 35, 30, 25, 20, 17, 15, 14, 13, 12, 11 or 10 amino acids.

Non-limiting examples of therapeutic protein or peptides include an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphoring, a semaphorin receptor, a serotonin transport protein, a serotonin uptake inhibitor, a serotonin receptor, a serpin, a serpin receptor, and a tumor suppressor. In certain embodiments, the therapeutic protein or peptide is selected from brain-derived neurotrophic factor (BDNF), ciliary neurotrophic factor (CNTF), macrophage colony-stimulating factor (CSF), epidermal growth factor (EGF), fibroblast growth factor (FGF), gonadotropin, interferon-gamma (IFN), insulin-like growth factor 1 (IFG-1), nerve growth factor (NGF), platelet-derived growth factor (PDGF), pigment epithelium-derived factor (PEDF), transforming growth factor (TGF), transforming growth factor-beta (TGF-B), tumor necrosis factor (TNF), vascular endothelial growth factor (VEGF), prolactin, somatotropin, X-linked inhibitor of apoptosis protein 1 (XIAP1), interleukin 1 (IL-1), IL-2, IL-3, IL-4, IL-5, IL-6, IL-7, IL-8, IL-9, IL-10, IL-10, viral IL-10, IL-11, IL-12, IL-13, IL-14, IL-15, IL-16, IL-17, and IL-18.

A therapeutic gene expression product can comprise gene editing components. Non-limiting examples of gene editing components include those required for CRISPR/Cas, artificial site-specific RNA endonuclease (ASRE), zinc finger endonuclease (ZFN), and transcription factor like effector nuclease (TALEN). In a non-limiting example, a subject having Huntington's disease is identified. The subject is then systemically administered a first amount of a rAAV encapsidating a viral vector encoding ZFN engineered to represses the transcription of the Huntingtin (HTT) gene. The rAAV will include a modified AAV capsid protein that includes an amino acid sequence provided in any one of Tables 1-3, FIG. 1 and Formulas I-VI, so as to allow proper targeting of the ZFN to the nervous system, while reducing expression in off-target organs, such as the liver. If needed, the subject is administered a second or third dose of the rAAV, until a therapeutically effective amount of the ZFN is expressed in the subject's nervous system.

A therapeutic nucleic acid can comprise a non-protein coding gene e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The non-protein coding gene may also encode a tRNA, IRNA, tmRNA, piRNA, double stranded RNA, snRNA, snoRNA, and/or long non-coding RNA (IncRNA). In some cases, the non-protein coding gene can modulate the expression or the activity of a target gene or gene expression product. For example, the RNAs described herein may be used to inhibit gene expression in the brain. In some cases, inhibition of gene expression refers to an inhibition by at least about 20%, 30%, 40%, 50%, 60%, 70%, 80%, 85%, 90%, 95% and 100%. In some cases, the protein product of the targeted gene may be inhibited by at least about 20%, 30%, 40%, 50%, 60%, 70%, 80%, 85%, 90%, 95% and 100%. The gene can be either a wild type gene or a gene with at least one mutation. The targeted protein may be either a wild type protein or a protein with at least one mutation.

A therapeutic nucleic acid can modulate the expression or activity of a gene or gene expression product expressed from the gene that is implicated in a disease or disorder of the brain. For example, the therapeutic nucleic acid, in some cases is a gene or a modified version of the gene described herein. In some instances, the gene or gene expression product is inhibited. In some instances, the gene or gene expression product is enhanced.

In another example, the therapeutic nucleic acid comprises an effector gene expression product such as a gene editing component specific to target a gene therein. Non-limited examples of genes include target gene or gene expression product selected from ATP1A2, CACNAIA, SETD5, SHANK3, NF2, DNMT1, TCF4, RAI1, PEX1, ARSA, EIF2B5, FIF2B1, FIF2B2, NPCl, ADAR, MFSD8, STXBPl, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMN1), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMR1), NPC Intracellular Cholesterol Transporter 1 (NPC1), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11β, PEX12, PEX13, PEX14, PEX16, PEX19, and PEX26. In some instances, the gene or gene expression product is inhibited. In some instances, the gene or gene expression product is enhanced.

### AAV Vectors

Aspects disclosed herein comprise plasmid vectors comprising a nucleic acid sequence encoding the AAV capsids and AAV capsid proteins described herein. AAV vectors described herein are useful for the assembly of a rAAV and viral packaging of a heterologous nucleic acid. In addition, an AAV vector may encode a transgene comprising the heterologous nucleic acid.

An AAV vector can comprise a transgene, which in some cases encodes a heterologous gene expression product (e.g., therapeutic gene expression product, recombinant capsid protein, and the like). The transgene is in cis with two inverted terminal repeats (ITRs) flanking the transgene. The transgene may comprise a therapeutic nucleic acid encoding a therapeutic gene expression product. Due to the limited packaging capacity of the rAAV (˜5 kB), in some cases, a longer transgene may be split between two AAV vectors, the first with 3′ splice donor and the second with a 5′ splice acceptor. Upon co-infection of a cell, concatemers form, which are spliced together to express a full-length transgene.

A transgene is generally inserted so that its expression is driven by the endogenous promoter at the integration site, namely the promoter that drives expression of the endogenous gene into which a transgene is inserted. In some instances, a transgene comprises a promoter and/or enhancer, for example a constitutive promoter or an inducible or tissue/cell specific promoter. As a non-limiting example, the promoter may be CMV promoter, a CMV-β-Actin-intron-β-Globin hybrid promoter (CAG), CBA promoter, FRDA or FXN promoter, UBC promoter, GUSB promoter, NSE promoter, Synapsin promoter, MeCP2 promoter, GFAP promoter, H1 promoter, U6 promoter, NFL promoter, NFH promoter, SCN8A promoter, or PGK promoter. As a non-limiting example, promoters can be tissue-specific expression elements include, but are not limited to, human elongation factor 1α-subunit (EF1α), immediate-early cytomegalovirus (CMV), chicken β-actin (CBA) and its derivative CAG, the β glucuronidase (GUSB), and ubiquitin C (UBC). The transgene may include a tissue-specific expression elements for neurons such as, but not limited to, neuron-specific enolase (NSE), platelet-derived growth factor (PDGF), platelet-derived growth factor B-chain (PDGF-β), the synapsin (Syn), the methyl-CpG binding protein 2 (MeCP2), Ca2+/calmodulin-dependent protein kinase II (CaMKII), metabotropic glutamate receptor 2 (mGluR2), NFL, NFH, np32, PPE, Enk and EAAT2 promoters. The transgene may comprise a tissue-specific expression element for astrocytes such as, but not limited to, the glial fibrillary acidic protein (GFAP) and EAAT2 promoters. The transgene may comprise tissue-specific expression elements for oligodendrocytes such as, but not limited to, the myelin basic protein (MBP) promoter.

In some embodiments, the promoter is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. The promoter may provide expression of the therapeutic gene expression product for a period of time in targeted tissues such as, but not limited to. the brain. Expression of the therapeutic gene expression product may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years. Expression of the payload may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years or 10-15 years, or 15-20 years, or 20-25 years, or 25-30 years, or 30-35 years, or 35-40 years, or 40-45 years, or 45-50 years, or 50-55 years, or 55-60 years, or 60-65 years.

An AAV vector can comprise a genome of a helper virus. Helper virus proteins are required for the assembly of a recombinant AAV (rAAV), and packaging of a transgene containing a heterologous nucleic acid into the rAAV. The helper virus genes are adenovirus genes E4, E2a and VA, that when expressed in the cell, assist with AAV replication. In some embodiments, an AAV vector comprises E2. In some embodiments, an AAV vector comprises E4. In some embodiments, an AAV vector comprises VA. In some instances, the AAV vector comprises one of helper virus proteins, or any combination.

The target gene or gene expression product for use in a transgene can be selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMN1), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMR1), NPC Intracellular Cholesterol Transporter 1 (NPC1), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11β, PEX12, PEX13, PEX14, PEX16, PEX19, and PEX26.

An AAV vector can comprise a viral genome comprising a nucleic acid encoding the recombinant AAV (rAAV) capsid protein described herein. The viral genome can comprise a Replication (Rep) gene encoding a Rep protein, and Capsid (Cap) gene encoding an AAP protein in the first open reading frame (ORF1) or a Cap protein in the second open reading frame (ORF2). The Rep protein is selected from Rep78, Rep68, Rep52, and Rep40. In some instances, the Cap gene is modified encoding a modified AAV capsid protein described herein. A wild-type Cap gene encodes three proteins, VP1, VP2, and VP3. In some cases, VP1 is modified. In some cases, VP2 is modified. In some cases, VP3 is modified. In some cases, all three VP1-VP3 are modified. The AAV vector can comprise nucleic acids encoding wild-type Rep78, Rep68, Rep52, Rep40 and AAP proteins.

In some instances, the AAV9 VP1 gene provided in Table 4, is modified to include any one of sequences described to produce a variant AAV capsid by the methods described herein.

TABLE 4
VP1 Capsid Protein Nucleic Acid Sequences

| Identifier | Sequence |
|---|---|
| AAV9 > AY530579.1 | ATGGCTGCCGATGGTTATCTTCCAGATTGGCTCGAG |
| Adeno-associated virus 9 | CAACCTTAGTGAAGGAATTCGCGAGTGGTGGGCTTT |
| isolate hu.14 capsid prote | AAACCTGGAGCCCCTCAACCCAAGGCAAATCAACA [symbol] |
| VP1 (cap) gene, complet | ATCAAGACAACGCTCGAGGTCTTGTGCTTCCGGGTT |
| cds | AAATACCTTGGACCCGGCAACGGACTCGACAAGGG |
| | AGCCGGTCAACGCAGCAGACGCGGCGGCCCTCGAG |
| | CGACAAGGCCTACGACCAGCAGCTCAAGGCCGGAG |
| | AACCCGTACCTCAAGTACAACCACGCCGACGCCGA [symbol] |
| | TCCAGGAGCGGCTCAAAGAAGATACGTCTTTTGGG [symbol] |
| | CAACCTCGGGCGAGCAGTCTTCCAGGCCAAAAAGA [symbol] |
| | CTTCTTGAACCTCTTGGTCTGGTTGAGGAAGCGGCT [symbol] |
| | GACGGCTCCTGGAAAGAAGAGGCCTGTAGAGCAGT [symbol] |
| | CCTCAGGAACCGGACTCCTCCGCGGGTATTGGCAAA |
| | CGGGTGCACAGCCCGCTAAAAAGAGACTCAATTTC [symbol] |
| | TCAGACTGGCGACACAGAGTCAGTCCCAGACCCTC [symbol] |
| | CCAATCGGAGAACCTCCCGCAGCCCCCTCAGGTGT [symbol] |
| | GATCTCTTACAATGGCTTCAGGTGGTGGCGCACCAG |
| | GCAGACAATAACGAAGGTGCCGATGGAGTGGGTAG |
| | CCTCGGGAAATTGGCATTGCGATTCCCAATGGCTGG |
| | GGACAGAGTCATCACCACCAGCACCCGAACCTGGG [symbol] |
| | CTGCCCACCTACAACAATCACCTCTACAAGCAAAT [symbol] |
| | CAACAGCACATCTGGAGGATCTTCAAATGACAACG [symbol] |
| | TACTTCGGCTACAGCACCCCCTGGGGGTATTTTGAC [symbol] |
| | CAACAGATTCCACTGCCACTTCTCACCACGTGACTG |
| | AGCGACTCATCAACAACAACTGGGGATTCCGGCCTA |
| | GCGACTCAACTTCAAGCTCTTCAACATTCAGGTCAA |
| | AGGTTACGGACAACAATGGAGTCAAGACCATCGCC [symbol] |
| | TAACCTTACCAGCACGGTCCAGGTCTTCACGGACTC |
| | ACTATCAGCTCCCGTACGTGCTCGGGTCGGCTCACG |
| | GGCTGCCTCCCGCCGTTCCCAGCGGACGTTTTCATG [symbol] |
| | TCCTCAGTACGGGTATCTGACGCTTAATGATGGAAG |
| | AGGCCGTGGGTCGTTCGTCCTTTTACTGCCTGGAAT [symbol] |
| | TTCCCGTCGCAAATGCTAAGAACGGGTAACAACTT [symbol] |
| | AGTTCAGCTACGAGTTTGAGAACGTACCTTTCCATA |
| | AGCTACGCTCACAGCCAAAGCCTGGACCGACTAAT [symbol] |
| | ATCCACTCATCGACCAATACTTGTACTATCTCTCAA [symbol] |
| | ACTATTAACGGTTCTGGACAGAATCAACAAACGCTA |
| | AATTCAGTGTGGCCGGACCCAGCAACATGGCTGTC [symbol] |
| | GGGAAGAAACTACATACCTGGACCCAGCTACCGAC [symbol] |
| | CAACGTGTCTCAACCACTGTGACTCAAAACAACAA [symbol] |
| | GCGAATTTGCTTGGCCTGGAGCTTCTTCTTGGGCTC [symbol] |
| | AATGGACGTAATAGCTTGATGAATCCTGGACCTGCT |
| | GGCCAGCCACAAAGAAGGAGAGGACCGTTTCTTTC [symbol] |
| | TTGTCTGGATCTTTAATTTTTGGCAAACAAGGAACT [symbol] |
| | AAGAGACAACGTGGATGCGGACAAAGTCATGATAA |
| | AACGAAGAAGAAATTAAAACTACTAACCCGGTAGC |
| | CGGAGTCCTATGGACAAGTGGCCACAAACCACCAG [symbol] |
| | TGCCCAAGCACAGGCGCAGACCGGCTGGGTTCAAA [symbol] |
| | CAAGGAATACTTCCGGGTATGGTTTGGCAGGACAG [symbol] |
| | ATGTGTACCTGCAAGGACCCATTTGGGCCAAAATT [symbol] |
| | CACACGGACGGCAACTTTCACCCTTCTCCGCTGATG |
| | AGGGTTTGGAATGAAGCACCCGCCTCCTCAGATCCT |
| | TCAAAAACACACCTGTACCTGCGGATCCTCCAACG [symbol] |
| | CTTCAACAAGGACAAGCTGAACTCTTTCATCACCCA |
| | ATTCTACTGGCCAAGTCAGCGTGGAGATCGAGTGG [symbol] |
| | GCTGCAGAAGGAAAACAGCAAGCGCTGGAACCCGG |
| | GATCCAGTACACTTCCAACTATTACAAGTCTAATAA |
| | TTGAATTTGCTGTTAATACTGAAGGTGTATATAGTG [symbol] |
| | CCCCGCCCCATTGGCACCAGATACCTGACTCGTAAT |
| | GTAA |

[symbol]

indicates data missing or illegible when filed

### Methods of Producing rAAVs

Disclosed herein are methods of producing the AAV capsids comprising the AAV capsid proteins and viral vector encoding a therapeutic nucleic acid. The AAV capsid proteins are produced by introducing into a cell (e.g., immortalized stem cell) a first vector containing a transgene cassette flanked by inverted terminal repeat (ITR) sequences from a parental AAV virus (the transgene cassette has a promoter sequence that drives transcription of a heterologous nucleic acid in the nucleus of the target cell), a second vector encoding the AAV genome with a AAV capsid protein (encoding the AAV Rep gene as well as the modified Cap gene for the variant being produced), and a third vector encoding helper virus proteins, required for assembly of the AAV capsid structure and packaging of the transgene in the modified AAV capsid structure. The assembled AAV capsid can be isolated and purified from the cell using suitable methods known in the art.

The transgenes contained in a recombinant AAV (rAAV) vector and encapsidated by the AAV capsid proteins of the present disclosure are also provided herein. The transgenes disclosed herein are delivered to a subject for a variety of purposes, such as to treat a disease or condition in the subject. The transgene can be gene editing components that modulate the activity or expression of a target gene or gene expression product. Alternatively, the transgene is a gene encoding a therapeutic gene expression product that is effective to modulate the activity or expression of itself, or another target gene or gene expression product.

Aspects disclosed herein provide methods of manufacturing rAAV virus or virus particles comprising: (a) introducing into a cell a nucleic acid comprising: (i) first vector containing a transgene cassette flanked by inverted terminal repeat (ITR) sequences from a parental AAV virus (the transgene cassette has a promoter sequence that drives transcription of a heterologous nucleic acid in the nucleus of the target cell); (ii) a second vector encoding the AAV genome with a AAV capsid protein of the present invention; and (iii) a vector encoding helper virus proteins, required for assembly of the AAV capsid structure and packaging of the transgene in the modified AAV capsid structure; (b) expressing in the cell the AAV capsid protein described herein; (c) assembling an AAV particle comprising the AAV capsid proteins disclosed herein; and (d) packaging the AAV particle. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the 5′ ITR and the 3′ ITR are derived from an AAV2 serotype. In some instances, the 5′ ITR and the 3′ ITR are derived from an AAV5 serotype. In some instances, the 5′ ITR and the 3′ ITR are derived from an AAV9 serotype. In some instances, the first nucleic acid sequence and the second nucleic acid sequence are in trans. In some instances, the first nucleic acid sequence and the second nucleic acid sequence are in cis. In some instances, the first nucleic acid sequence, the second nucleic acid sequence and the third nucleic acid sequence, are in trans.

The Cap gene disclosed here comprises any one of sequences from Table 4, which are DNA sequences encoding the modified AAV capsid protein portions of the present disclosure.

In some instances, the methods comprise packing the first nucleic acid sequence encoding the therapeutic gene expression product such that it becomes encapsidated by the modified AAV capsid protein. In some embodiments, the rAAV particles are isolated, concentrated, and purified using suitable viral purification methods, such as those described herein.

In some cases, rAAVs of the present disclosure are generated using the methods described in Challis, R. C. et al. Nat. Protoc. 14, 379 (2019). Briefly, triple transfection of HEK293T cells (ATCC) using polyethylenimine (PEI) is performed, viruses are collected after 120 hours from both cell lysates and media and purified over iodixanol. In a non-limiting example, the rAAVs are generated by triple transfection of precursor cells (e.g., HEK293T) cells using a standard transfection protocol (e.g., PEI). Viral particles are harvested from the media after a period of time (e.g., 72 h post transfection) and from the cells and media at a later point in time (e.g., 120 h post transfection). Virus present in the media is concentrated by precipitation with 8% polyethylene glycol (PEG) and 500 mM sodium chloride and the precipitated virus is added to the lysates prepared from the collected cells. The viruses are purified over iodixanol (Optiprep, Sigma) step gradients (15%, 25%, 40% and 60%). Viruses are concentrated and formulated in PBS. Virus titers are determined by measuring the number of DNasel-resistant vector genome copies (VGs) using qPCR and the linearized genome plasmid as a control.

The cell can be selected from a human, a primate, a murine, a feline, a canine, a porcine, an ovine, a bovine, an equine, an epine, a caprine and a lupine host cell. In some instances, the cell is a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some cases, the immortalized cell is a HEK293cell. In some instances, the cell is a differentiated cell. Based on the disclosure provided, it is expected that this system can be used in conjunction with any transgenic line expressing a recombinase in the target cell type of interest to develop AAV capsids that more efficiently transduce that target cell population.

### Methods of Treatment

Disclosed herein are methods of treating a disease or condition, or a symptom of the disease or condition, in a subject, comprising administrating of therapeutically effective amount of one or more compositions (e.g., rAAV particle, AAV vector, pharmaceutical composition) disclosed herein to the subject. In some embodiments, the composition is a rAAV capsid protein described herein. In some embodiments, the composition is an isolated and purified rAAV capsid protein described herein. In some embodiments, the rAAV particle encapsidates an AAV vector comprising a transgene (e.g., therapeutic nucleic acid). In some embodiments, the composition is a rAAV capsid protein described herein conjugated with a therapeutic agent disclosed herein. In some embodiments, the composition is a pharmaceutical composition comprising the rAAV particle and a pharmaceutically acceptable carrier. In some embodiments, the one or more compositions are administered to the subject alone (e.g., stand-alone therapy). In some embodiments, the composition is a first-line therapy for the disease or condition. In some embodiments, the composition is a second-line, third-line, or fourth-line therapy, for the disease or condition.

Recombinant adeno-associated virus (rAAV) mediated gene delivery leverages the AAV mechanism of viral transduction for nuclear expression of an episomal heterologous nucleic acid (e.g., a transgene, therapeutic nucleic acid). Upon delivery to a host in vivo environment, a rAAV will (1) bind or attach to cellular surface receptors on the target cell, (2) endocytose, (3) traffic to the nucleus, (4) uncoat the virus to release the encapsidated heterologous nucleic acid, (5) convert of the heterologous nucleic acid from single-stranded to double-stranded DNA as a template for transcription in the nucleus, and (6) transcribe of the episomal heterologous nucleic acid in the nucleus of the host cell (“transduction”). rAAVs engineered to have an increased transduction enrichment (transcription of the episomal heterologous nucleic acid in the host cell) are desirable for gene therapy applications.

Aspects disclosed herein provide methods of treating a disease or condition in a subject, the method comprising administering to the subject a therapeutically effective amount of the rAAV of the present disclosure, or the pharmaceutical formulation of the present disclosure, wherein the gene product is a therapeutic gene product. In some embodiments, the administering is by intracranial, intraventricular, intracerebroventricular, intravenous, intraarterial, intranasal, intrathecal, intracisternae magna, or subcutaneous.

Provided here, are methods of treating a disease or a condition associated with an aberrant expression or activity of a target gene or gene expression product thereof, the method comprising modulating the expression or the activity of a target gene or gene expression product in a subject by administering a rAAV encapsidating a heterologous nucleic acid of the present disclosure. In some instances, the expression or the activity of the target gene or gene expression product is decreased, relative to that in a normal (non-diseased) individual; and administering the rAAV to the subject is sufficient to increase the expression of the activity of the target gene or gene expression product. In some instances, the expression or the activity of the gene or gene expression product is increased, relative to that in a normal individual; and administering the rAAV to the subject is sufficient to decrease the expression or the activity of the target gene or gene expression product. In a non-limiting example, a subject diagnosed with Alzheimer's disease, which is caused, in some cases, by a gain-of-function of a Presenilin 1 and/or Presenilin 2 (encoded by the gene PSEN1 and PSEN2, respectively) is administered a rAAV disclosed herein encapsidating a therapeutic nucleic acid that is a silencing RNA (siRNA), or other RNAi with a loss-of-function effect on PSENI mRNA.

Also provided are methods of preventing a disease or condition disclosed herein in a subject comprising administering to the subject a therapeutically effective amount of an rAAV vector comprising a nucleic acid sequence encoding a therapeutic gene expression product described herein. The rAAV vector may be encapsidated in the modified capsid protein or rAAV viral particle described herein. In some instances, the therapeutic gene expression product is effective to modulate the activity or expression of a target gene or gene expression product.

Disclosed herein are methods of treating a disease or condition in a subject by administering a composition comprising a rAAV disclosed herein. An advantage of the rAAVs disclosed herein, is that the rAAV may be used to treat virtually any disease or condition that would benefit from a transgene therapy, including but not limited to spinal muscular atrophy (SMA), amyotrophic lateral sclerosis (ALS), Parkinson's disease, Pompe disease, mucopolysaccharidosis type II, fragile X syndrome, STXBPI encephalopathy, Krabbe disease, Huntington's disease, Alzheimer's disease, Battens disease, lysosomal storage disorders, glioblastoma multiforme, Rett syndrome, Leber's congenital amaurosis, Late infantile neuronal ceroid lipofuscinosis (LINCL), chronic pain, stroke, spinal cord injury, traumatic brain injury and lysosomal storage disorders.

In some cases, the disease or condition is localized to a particular in vivo environment in the subject, e.g., the brain. The compositions of the present disclosure are particularly useful for the treatment of the diseases or conditions described herein because they specifically or more efficiently target the in vivo environment and deliver a therapeutic nucleic acid engineered to modulate the activity or the expression of a target gene expression product involved with the pathogenesis or pathology of the disease or condition.

Provided herein are methods of treating a disease or a condition, or a symptom of the disease or condition, in a subject, comprising: (a) diagnosing a subject with a disease or a condition affecting a target in vivo environment; and (b) treating the disease or the condition by administering to the subject a therapeutically effective amount of a composition disclosed herein (e.g., rAAV particle, AAV vector, pharmaceutical composition), wherein the composition is engineered with an increased specificity for the target in vivo environment.

Disclosed herein are methods of treating a disease or a condition, or a symptom of the disease or the condition, afflicting a target in a subject comprising: (a) administering to the subject a composition (e.g., rAAV particle, AAV vector, pharmaceutical composition); and (b) expressing the therapeutic nucleic acid into a target in vivo environment in the subject with an increased transduction enrichment.

In some embodiments, methods further comprise reducing or ablating delivery of the heterologous nucleic acid in an off-target in vivo environment, such as the liver. In some embodiments, delivery is characterized by an increase in enrichment of transduction (e.g., of the heterologous nucleic acid) in the brain.

In some embodiments, methods of treating a disease or condition affecting the brain comprise administering a rAAV particle to a brain in a subject, the rAAV particle comprising an rAAV capsid protein comprising an insertion of about, five, six, or seven amino acids of an amino acid sequence provided in Tables 1-3, FIG. 1 and Formulas I-VI, at an amino acid position 588-589 in a parental AAV capsid protein. In some embodiments, methods of treating a disease or condition affecting the brain comprise administering a rAAV particle to a brain in a subject, the rAAV particle comprising an rAAV capsid protein comprising an insertion of about, five, six, or seven amino acids of an amino acid sequence as well as one or more substitution at amino acid found at amino acid positions 587-590 [AQAQ] such as provided in Tables 1-3, FIG. 1 and Formulas I-VI. In some embodiments, the parental AAV capsid protein is AAV9 capsid protein (for e.g., provided in SEQ ID NO: 1.

Also provided are methods of modulating a target gene expression product, the methods comprising administering to a subject in need thereof a composition (e.g., rAAV particle, AAV vector, pharmaceutical composition) disclosed herein. For example, methods provided herein comprise administering to a subject a rAAV with a rAAV capsid protein encapsidating a viral vector comprising a heterologous nucleic acid that modulates the expression or the activity of the target gene expression product.

The term “normal individual” refers to an individual that is not afflicted with the disease or the condition characterized by the variation in expression or activity of the gene or gene expression product thereof.

In some embodiments, the disease or condition of the brain selected from Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavemous Malformation, Cerebral Gigantism, Cerebral Hypoxia, Cerebral Palsy, Cerebro-Oculo-Facio-Skeletal Syndrome (COFS), Charcot-Marie-Tooth Disease, Charcot-Marie-Tooth syndrome, classical rhizomelic chondrodysplasia punctata (RCDP), Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, Deafness, De Morsier's Syndrome, Dejerine-Klumpke Palsy, Dementia, Dementia-Multi-Infarct, Dementia-Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Duchenne muscular dystrophy, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X syndrome, Friedreich's Ataxia, Frontotemporal Dementia (FTD), Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, glioblastoma, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus-Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease (IRD), Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus-Neurological Sequelae, Lyme Disease-Neurological Complications, Machado-Joseph Disease, Macrencephaly, Maple syrup urine disease, Megalencephaly, Melkersson-Rosenthal Syndrome, Meningitis, Meningitis and Encephalitis, Menkes Disease, Menkes syndrome, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Mucopolysaccharidosis II, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Myotonic dystrophy, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O′Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phenylketonuria, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Prader-Willi syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar ataxia, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, STXBPI encephalopathy, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tangier disease, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central Nervous Systems, Von Economo's Disease, Von Hippel-Lindau Disease (VHL), Von Hippel-Lindau syndrome, Von Recklinghausen's Discase, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy and Zellweger syndrome.

In some embodiments, the pharmaceutical formulation comprises a therapeutic nucleic acid encoding a therapeutic gene expression product. In some instances, the therapeutic gene expression product is effective to modulate an activity or an expression of a target gene or gene expression product selected from ATP1A2, CACNAIA, SETD5,SHANK3, NF2, DNMT1, TCF4, RAI1, PEX1, ARSA, FIF2B5, FIF2B1, EIF2B2, NPC1, ADAR, MFSD8, STXBP1, PRICKLE2, PRRT2, IDUA, STXIB, Sarcoglycan Alpha (SGCA), glutamic acid decarboxylase 65 (GAD65), glutamic acid decarboxylase 67 (GAD67), CIN2, Nerve Growth Factor (NGF), glial cell derived neurotrophic factor (GDNF), Survival Of Motor Neuron 1, STXBP1, Telomeric (SMN1), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMR1), NPC Intracellular Cholesterol Transporter 1 (NPC1), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11β, PEX12, PEX13, PEX14, PEX16, PEX19, and PEX26.

In some aspects, other examples of genes involved in neurologic or brain diseases or disorders include MAPT, IDUA, SNCA, ATXN2, Ube3a, GNS, HGSNAT, NAGLU, SGSH, CLN1, CLN3, CLN4, CLN5, CLN6, CLN7, CLN8, CTSD, ABCD1, HEXA, HEXB, ASM, ASPA, GLB1, AADC, MFN2, GNAO1, SYNGAP1, GRIN2A, GRIN2B, KCNQ2, EPM2A, NHLRC1, SLC6A1, SLC13A5, SURF1, GBE1, ATXN1, ATXN3, and ATXN7.

In some instances, the therapeutic gene expression product comprises gene editing components. In some instances, the gene editing components are selected from an artificial site-specific RNA endonuclease (ASRE), a zinc finger endonuclease (ZFN), a transcription factor like effector nuclease (TALEN), a clustered regularly interspaced short palindromic repeats (CRISPR)/Cas enzyme, and a CRISPR)/Cas guide RNA.

In some instances, the expression of a gene or expression or activity of a gene expression product is inhibited by the administration of the composition to the subject. In some instances, the expression of a gene or the expression or the activity of a gene expression product is enhanced by the administration of the composition to the subject.

### Formulations, Dosages, and Routes of Administration

Disclosed herein are methods comprising delivering a rAAV particle encapsidating a heterologous nucleic acid to the brain in a subject, the rAAV particle comprising (i) an increased transduction of the heterologous nucleic acid in the brain, wherein the rAAV particle has an rAAV capsid protein comprising an insertion of five, six, or seven amino acids of an amino acid sequence provided in Tables 1-3, FIG. 1 and Formulas I-VI, at an amino acid position 588-589 in a parental AAV capsid protein as well as one or more substitution at amino acid found at amino acid positions 587-590 [AQAQ] such as provided in Tables 1-3, FIG. 1 and Formulas I-VI. In various embodiments, the rAAV capsid protein may comprise one or more substitutions at amino acid positions 452-458 alone or in combination with the modifications above.

In general, methods disclosed herein comprise administering a therapeutic rAAV composition by systemic administration. In some instances, methods comprise administering a therapeutic rAAV composition by intravenous (“i.v.”) administration. One may administer therapeutic rAAV compositions by additional routes, such as subcutaneous injection, intramuscular injection, intradermal injection, transdermal injection, percutaneous administration, intranasal administration, intralymphatic injection, rectal administration intragastric administration, intraocular administration, intracerebroventricular administration, intrathecally, intracisternal, or any other suitable parenteral administration. Routes, dosage, time points, and duration of administrating therapeutics may be adjusted. In some embodiments, administration of therapeutics is prior to, or after, onset of either, or both, acute and chronic symptoms of the disease or condition. Other routes of delivery to the brain include, but are not limited to intracranial administration, lateral cerebroventricular administration, and endovascular administration.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some cases, a dose of the pharmaceutical composition may comprise a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases, the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, or 2×10¹⁷. In some cases, the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, or 3×10¹⁷. In some cases, the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, or 4×10¹⁷. In some cases, the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, or 5×10¹⁷. In some cases, the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, or 6×10¹⁷. In some cases, the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10¹⁶, or 7×10¹⁷. In some cases, the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, or 8×10¹⁷. In some cases, the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, or 9×10¹⁷.

Disclosed herein, in some embodiments are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the pharmaceutical forms of the rAAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

In some cases, for administration of an injectable aqueous solution, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous or intrathecal administration.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of rAAV compositions and time of administration of such compositions will be within the purview of the skilled artisan having benefit of the present teachings. It is likely, however, that the administration of therapeutically-effective amounts of the disclosed compositions may be achieved by a single administration, example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In certain embodiments, the data obtained from cell culture assays and animal studies are used in formulating the therapeutically effective daily dosage range and/or the therapeutically effective unit dosage amount for use in mammals, including humans. In certain embodiments, the dosage range and/or the unit dosage amount varies within this range depending upon the dosage form employed and the route of administration utilized.

### Combination Therapies

A therapeutic rAAV may be used alone or in combination with an additional therapeutic agent (together, “therapeutic agents”). In some cases, a therapeutic rAAV as used herein is administered alone. The therapeutic agent may be administered together or sequentially in a combination therapy. The combination therapy may be administered within the same day, or may be administered one or more days, weeks, months, or years apart.

The additional therapeutic agent can comprise a small molecule. The additional therapeutic agent can comprise an antibody, or antigen-binding fragment. The additional therapeutic agent can include lipid nanoparticle-based therapies, anti-sense oligonucleotide therapies, as well as other viral therapies.

The additional therapeutic agent can comprise a cell-based therapy. Exemplary cell-based therapies include without limitation immune effector cell therapy, chimeric antigen receptor T-cell (CAR-T) therapy, natural killer cell therapy and chimeric antigen receptor natural killer (NK) cell therapy. Either NK cells, or CAR-NK cells, or a combination of both NK cells and CAR-NK cells can be used in combination with the methods disclosed herein. In some embodiments, the NK cells and CAR-NK cells are derived from human induced pluripotent stem cells (iPSC), umbilical cord blood, or a cell line. The NK cells and CAR-NK cells can comprise a cytokine receptor and a suicide gene. The cell-based therapy can comprise a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

### Kits

Disclosed herein are kits comprising compositions disclosed herein. Also disclosed herein are kits for the treatment or prevention of a disease or conditions of the brain. In some instances, the disease or condition is cancer, a pathogen infection, pulmonary disease or condition, neurological disease, muscular disease, or an immune disorder, such as those described herein.

In one embodiment, a kit can include a therapeutic or prophylactic composition containing an effective amount of a composition of a rAAV particle encapsidating a recombinant AAV vector encoding a therapeutic nucleic acid (e.g., therapeutic nucleic acid) and a recombinant AAV (rAAV) capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some instances, the kit further comprises a cell. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the kit further comprises an AAV vector comprising a heterologous nucleic acid encoding a therapeutic gene expression product. In some instances, the AAV vector is an episome.

In some cases, rAAV are provided together with instructions for administering the rAAV to a subject having or at risk of developing the disease or condition (e.g., disease of the brain). Instructions can generally include information about the use of the composition for the treatment or prevention of the disease or condition.

In some cases, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some cases, instructions provide procedures for administering the rAAV to the subject alone. In some instances, the instructions provide that the rAAV is formulated for systemic delivery.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acne, eczema, psoriasis, and rosacea.

The terms “homologous,” “homology,” or “percent homology” are used herein to generally mean an amino acid sequence or a nucleic acid sequence having the same, or similar sequence to a reference sequence. Percent homology of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “subject” is any organism. In some instances, the organism is a mammal. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a “non-human primate” is a mammal, for example a monkey. In some instances, the subject is a patient, which as used herein, may refer to a subject diagnosed with a particular disease or disorder.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence.

The term “adeno-associated virus,” or “AAV” as used herein refers to the adeno-associated virus or derivatives thereof. Non-limited examples of AAV's include AAV type 1(AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), avian AAV, bovine AAV, canine AAV, equine AAV, primate AAV, non-primate AAV, and ovine AAV. In some instances, the AAV is described as a “Primate AAV,” which refers to AAV that infect primates. Likewise an AAV may infect bovine animals (e.g., “bovine AAV”, and the like). In some instances, the AAV is wildtype, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a transgene, therapeutic nucleic acid, viral genome). In some instances, the AAV capsid of the instant disclosure is a modified AAV capsid, relative to a corresponding parental AAV capsid protein.

The term “tropism” as used herein refers to a quality or characteristic of the AAV capsid that may include specificity for, and/or an increase or a decrease in enrichment of, expressing the encapsidated genetic information into an in vivo environment, relative to a second in vivo environment. An in vivo environment, in some instances, is a cell-type. An in vivo environment, in some instances, is an organ or organ system

The term “AAV vector” as used herein refers to nucleic acid polymer encoding genetic information related to the virus. The AAV vector may be a recombinant AAV vector (rAAV), which refers to an AAV vector generated using recombinatorial genetics methods. In some instances, the rAAV vector comprises at least one heterologous polynucleotide (e.g. a polynucleotide other than a wild-type or naturally occurring AAV genome such as a transgene).

The term “AAV particle” as used herein refers to an AAV virus, virion, AAV capsid protein or component thereof. In some cases, the AAV particle is modified relative to a parental AAV particle.

The term “gene product” of “gene expression product” refers to an expression product of a polynucleotide sequence such as, for e.g., a polypeptide, peptide, protein or RNA, including interfering RNA (e.g., siRNA, miRNA, shRNA) and messenger RNA (mRNA).

The term “heterologous” as used herein refers to a genetic element (e.g., coding region) or gene expression product (e.g., RNA, protein) that is derived from a genotypically distinct entity from that of the rest of the entity to which it is being compared.

The term “endogenous” as used herein refers to a genetic element (e.g., coding region) or gene expression product (e.g., RNA, protein) that is naturally occurring in or associated with an organism or a particular cell within the organism.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, systemic administration.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. Alternatively, a sample can be obtained through primary patient derived cell lines, or archived patient samples in the form of preserved samples, or fresh frozen samples.

The term “in vivo” is used to describe an event that takes place in a subject's body.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “brain” means a tissue selected from brain, thalamus, cortex, putamen, lateral ventricles, medulla, the pons, the amygdala, the motor cortex, caudate, hypothalamus, striatum, ventral midbrain, neocortex, basal ganglia, hippocampus, cerebrum, cerebellum, brain stem, and spinal cord. The brain includes a variety of cortical and subcortical areas, including the frontal, temporal, occipital and parietal lobes.

The term “systemic delivery” is defined as a route of administration of medication or other substance into a circulatory system so that the entire body is affected. Administration can take place via enteral administration (absorption of the drug through the gastrointestinal tract) or parenteral administration (generally injection, infusion, or implantation). “Circulatory system” includes both blood or cerebrospinal fluid circulatory systems. Examples of systemic administration for the brain include intraarterial, intravenous or intrathecal injection. Other examples include administration to the cerebrospinal fluid at any location, in the spine (i.e. but not limited to lumbar) or brain (i.e. but not limited to cisterna magna). The terms “systemic administration” and “systemic delivery” are used interchangeably.

The section headings used herein are for organizational purposes only and are not to be construed as limiting the subject matter described.

### EXAMPLES

### Example 1

### Method of Identifying the Modified Capsid Proteins in Cynomolgus Macaques

Of primary concern for the therapeutic applicability of engineered adeno-associated viruses (AAVs) is how well their transduction profiles translate to human application. While previous engineering efforts have focused on in vitro or in vivo rodent screening platforms due to the ease and flexibility of their use, screening efforts directly in non-human primates (NHPs) are much more likely to identify viruses that translate. We chose cynomolgus macaques, an old world NHP, for our engineering efforts. We focused our engineering efforts on a region of the AAV9 capsid surface located at amino acid position 588, one of the most exposed loops on the capsid surface that is a variable region between natural AAV serotypes and has a role in receptor binding. Insertion of peptides between positions 588 and 589 has been studied in the past by us, and others, and has resulted in novel receptor binding (AAV-PHP.B/AAV-PHP.eB binding of Ly6a on rodent brain endothelium to facilitate blood-brain barrier crossing and high transduction of the brain) and drastically altered capsid tropism. We chose to create a library of viral capsid by performing a random 7 amino acid insertion at this site within AAV9, hoping for novel tropism toward the NHP brain.

Plasmids. The first-round viral DNA library was generated by amplification of a section of the AAV9 capsid genome between amino acids 450-599 using NNK degenerate primers (Integrated DNA Technologies, Inc., IDT) to insert seven random amino acids between amino acids 588 and 589 with all possible variations. The resulting library inserts were then introduced into the rAAV-ACap-in-rev-RNA plasmid via Gibson assembly as previously described. The resulting capsid DNA library, rAAV-Cap-Cag-GFP11, contained a diversity of ˜1.28 billion variants at the amino acid level. The second round viral DNA library was generated similarly to the first round, but instead of NNK degenerate primers inserted at the 588, a synthesized oligo pool (Twist Biosicence) was used to generate only selected variants in a UBC-Cap-DNA and CAG-Cap-DNA construct with CAP. This second-round DNA library contained a diversity of 20,000 variants at the amino acid level with 2-3 barcoded replicates for each variant.

The AAV2/9 REP-AAP-ACAP plasmid transfected into HEK293T cells to provide the Rep gene for library viral production prevents production of a wild-type AAV9 capsid during viral library production after a plausible recombination event between this plasmid co-transfected with the library plasmids at each stage containing the library inserts.

Viral production. Recombinant AAVs were generated according to established protocols. Briefly, immortalized HEK293T cells (ATCC) were quadruple transfected with four vectors using polyethylenimine (PEI). The first vector was the rAAV-Cap-in-cis-Lox library flanked by inverted terminal repeat (ITR) sequences from a parental AAV virus. The second vector was the AAV2/9 REP-AAP-ACAP plasmid. The third vector contains nucleic acids encoding helper virus proteins needed for viral assembly and packaging of the heterologous nucleic acid into the modified capsid structure. The fourth is a pUC-18 plasmid included to achieve the right PEI/DNA ratio for optimal transfection enrichment. Only 10 ng of rAAV-Cap-in-cis-Lox library DNA was transfected (per 150 mm plate) to decrease the likelihood of multiple library DNAs entering the same cell. Viral particles are harvested from the cells and media after 60 h post transfection. Virus present in the media is concentrated by precipitation with 8% polyethylene glycol and 500 mM sodium chloride and the precipitated virus is added to the lysates prepared from the collected cells. The viruses are purified over iodixanol (Optiprep, Sigma) step gradients (15%, 25%, 40%, and 60%). Viruses are concentrated and formulated in PBS. Virus titers are determined by measuring the number of DNasel-resistant vector genome copies (VGs) using qPCR and the linearized genome plasmid as a control.

Animals. Cynomolgus Macaque procedures were approved by ACUC of the National Institutes of Mental Health. Cynomolgus Macaques were born and raised in NIMH colonies and housed in family groups under standard conditions of 27° C. temperature and 50% humidity. They were fed ad libitum and received enrichment as part of the primate enrichment program for NHPs at the NIH. For AAV infusions, animals were screened for endogenous neutralizing antibodies (Nab). None of the animals that were screened showed any detectible blocking reaction at 1:5 dilution of serum (Penn Vector Core, University of Pennsylvania). They were then housed individually for several days and acclimated to a new room before injections. 5 infant and juvenile monkeys were used for each, three each for first and 2 each for second round libraries. The day before infusion the animals' food was removed. Animals were anesthetized with isoflurane in oxygen, the skin over the femoral vein was shaved and sanitized with an isopropanol scrub, and the virus was infused over several minutes. Anesthesia was withdrawn and the animals were monitored until they became active, upon which they were returned to their cages. Activity and behavior were closely monitored over the next three days, with daily observations thereafter.

DNA/RNA recovery and sequencing. Viral libraries were injected into marmosets at a dose of 1×10¹³ vg/kg animal and rAAV genomes were recovered three weeks post injection. Animals were euthanized and brain, spinal cord and liver were recovered, snap frozen, and placed into long-term storage at −80° C. as well as other peripheral tissues such as heart, spleen, adrenal, kidney, and quad. The brain was separated into eleven brain regions, and 20-300 mg of each brain section was homogenized in buffer using the MagMAX DNA ULTRA (A25597) and a Bead Ruptor 96 (OMNI, INC) and viral DNA was isolated according to the manufacturers recommended protocol. Recovered viral DNA was treated with RNase, and purified with a Zymo DNA Clean and Concentrator kit (D4033). Viral genomes were enriched by 25 cycles of PCR amplification with primers flanking the 588-589 insertion site in the capsid genome using 50% of the total extracted viral DNA as a template. After Zymo DNA purification, samples were diluted 1:10 to 1:1000 depending on tissue type and each dilution further amplified around the library variable region with 10 cycles of PCR. Subsequently, samples were further amplified using custom primers with Illumina Indices for 10 more cycles. The amplification products were run on a 2% low-melting point agarose gel (ThermoFisher Scientific, 16520050) for better separation and recovery of the 210 bp band.

For the second round library only, packaged viral library DNA was isolated from the injected viral library by digestion of the viral capsid and purification of the contained ssDNA. These viral genomes were amplified by two PCR amplification steps, like the viral DNA extracted from tissue, to add adapters and indices for Illumina next-generation sequencing, and purified after gel electrophoresis. This viral library DNA, along with the viral DNA extracted from tissue, was sent for deep sequencing using an Illumina NextSeq 2000 system.

NGS data alignment and processing. Raw fastq files from NGS runs were processed with custom-built scripts (Capsida CapSeq Tools). For the first round library, the pipeline to process these datasets involved filtering to remove low-quality reads, utilizing a quality score for each sequence, and eliminating bias from PCR-induced mutations or high GC-content. The filtered dataset was then aligned by a perfect string match algorithm and trimmed to improve the alignment quality. Read counts for each sequence were pulled out and displayed by tissue, at which point all sequences found in the brain were compiled for formation of the second round library.

For the second round library read counts by tissue were similarly tabulated. Then, a read count of 1 was added to each sequence to remove 0 values, all brain regions for cach sequence were summed together, and the read sequences for each codon replicate of a given 7-mer amino acid sequence were summed together to give a single value for each peptide insertion. Finally, the data was log10 counts per million (Cpm) normalized. Enrichment values were calculated with normalized cpm of brain vs cpm of virus and converted to log10.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

1. An AAV capsid protein comprising an insertion sequence at position 588 as provided in Table 1 or FIG. 1.

2. An AAV capsid protein comprising an amino acid substitution and insertion sequence at position 587-590 as provided in Table 1 or FIG. 1

3. An AAV capsid protein comprising an amino acid substitution sequence at position 452-458 as provided in Table 2 or FIG. 1.

4. An AAV capsid protein comprising an insertion sequence of Formula I at position 588
E-N-H-V-X¹-X²-X³ (I)
wherein X¹ is an amino acid selected from K and R; wherein X² is an amino acid selected from F, G, L, N, P, Q, R, S and V; and X³ is an amino acid selected from C, D, E, F, L, M, N, P, Q, R, S, T and V.

5. The AAV capsid protein of claim 4 wherein X³ is S.

6. The AAV capsid protein of claim 4 wherein X³ is R.

7. The AAV capsid protein of claim 4 wherein X³ is T.

8. The AAV capsid protein of claim 4 wherein X² is S.

9. The AAV capsid protein of claim 4 wherein X² is N.

10. The AAV capsid protein of claim 4 wherein X² is R, F or L.

11. The AAV capsid protein of claim 4 wherein X¹ is R.

12. The AAV capsid protein of claim 4 wherein X¹ is K.

13. The AAV capsid protein of claim 4 selected from ENHVRSR, ENHVKSS, ENHVRST, ENHVRNR, ENHVRRS, ENHVRFS, and ENHVRLS

14. An AAV capsid protein comprising an insertion sequence of Formula II at position 588
X⁴-X⁵-X⁶-X⁷-R-S-S (II)
wherein X⁴ is an amino acid selected from E, F, G, L, M, N, Q, R, S, T and V; X⁵ is an amino acid selected N and T; X⁶ is an amino acid selected F, H, I, K, M, N, Q, R, S, T and V; and X⁷ is an amino acid selected from I, N, T and V.

15. The AAV capsid protein of claim 14 wherein X⁵ is N.

16. AAV capsid protein of claim 14 wherein X⁷ is V.

17. The AAV capsid protein of claim 14 selected from ENHTRSS, VNHVRSS, ENNVRSS, ENSVRSS, ENHTRSS, ENHIRSS, ENKVRSS, ENRVRSS, FNHVRSS, and NNHVRSS.

18. An AAV capsid protein comprising an insertion sequence of Formula III at position 588
X⁸-NGTR-X⁹-X¹⁰ (III)
wherein X⁸ is an amino acid selected from G, N, S and T; wherein X⁹ is an amino acid selected from H, I, L, M, N, P, Q and T; and wherein X¹⁰ is an amino acid selected from I, L, M, N, Q, S and T.

19. The AAV capsid protein of claim 18 wherein X⁸ is G.

20. The AAV capsid protein of claim 18 wherein X⁸ is T.

21. The AAV capsid protein of claim 18 wherein X⁹ is P.

22. The AAV capsid protein of claim 18 wherein X⁹ is I or L.

23. The AAV capsid protein of claim 18 wherein X¹⁰ is I or T.

24. The AAV capsid protein of claim 18 wherein X¹⁰ is L or Q.

25. The AAV capsid protein of claim 18 selected from GNGTRPI, TNGTRPT, GNGTRLT, GNGTRIT, GNGTRPL, and GNGTRPQ.

26. The AAV capsid protein of claims 1-2 and 4-25 further comprising a sequence at AA 452-458 as provided in Table 2 or FIG. 1.

27. An AAV capsid protein comprising an insertion sequence of Formula IV at position at 452-458
X¹¹-X¹²-X¹³-X¹⁴-X¹⁵-N-Q (IV)
wherein X¹¹ is an amino acid selected from A, D, G, H, I, L, M, N, P, Q, S, V, W and Y; X¹² is an amino acid selected from G, L, N, P, S and V; X¹³ is an amino acid selected from F, I, N, and S; X¹⁴ is an amino acid selected from A, G, L, N and T; and X¹⁵ is an amino acid selected from F and Q.

28. The AAV capsid protein of claim 27 wherein X¹¹ is an amino acid selected from A, G, H, L, N, P, Q, S, V and W.

29. The AAV capsid protein of claim 27 wherein X¹² is G, N, P or V.

30. The AAV capsid protein of claim 27 wherein X¹³ is F, N or S.

31. The AAV capsid protein of Claim27 wherein X¹⁴ is G.

32. The AAV capsid protein of claim 27 wherein X¹⁵ is Q.

33. An AAV capsid protein comprising an amino acid sequence of Formula V at 452-458
X¹⁶-G-S-G-Q-N-Q (V)
wherein X³² is an amino acid selected from A, D, G, H, I, L, M, N, P, S, W and Y.

34. An AAV capsid comprising an amino acid sequence of Formula VI at 452-458
N-G-S-G-X¹⁷-X¹⁸-X¹⁹ (VI)
wherein X¹⁷ is an amino acid selected from F and Q; X¹⁸ is an amino acid selected from I, M and N; and X¹⁹ is an amino acid selected from Q and V.

35. The AAV capsid protein of claim 27 comprising a sequence at AA 452-458 selected from AGSGQNQ, DGSGQNQ, GGSGQNQ, HGSGQNQ, IGSGQNQ, LGSGQNQ, MGSGQNQ, NGFGQNQ, NGIGQNQ, NGNGQNQ, NGSAQNQ, NGSGENQ, NGSGQIQ, NGSGQMQ, NGSGQNQ, NGSGQNV, NGSLQNQ, NGSNQNQ, NGSTQNQ, NLSGQNQ, NSSGQNQ, PGSGQNQ, SGSGQNQ, VGSGQNQ, WGSGQNQ, and YGSGQNQ.

36. The AAV capsid protein of claim 27 comprising a sequence at AA 452-458 selected from DGSGQNQ, FGSGQNQ, GGSGQNQ, IGSGQNQ, MGSGQNQ, NGSAQNQ, NGSGQFG, NGSGQLQ, NGSGQNQ, NGSGQNV, NGSLQNQ, NGSMQNQ, NGSSQNQ, NGSTQNQ, PGSGQNQ, QGSGQNQ, SGSGQNQ, TGSGQNQ, VGSGQNQ, and WGSGQNQ.

37. The AAV capsid protein of claim 27 comprising a sequence at AA 452-458 selected from NGSGQNQ, NGSMQNQ, and NGKSSNQ.

38. An AAV capsid protein comprising substituting an amino acid sequence at 452-458 selected from AGSGQNQ, APSGQNQ, GGSGQNQ, HGSGQNQ, LGSGQNQ, NGNGQNQ, NVSGQNQ, NGSGQNQ, NGFGQNQ, NGSGQIQ, NSSGQNQ, NGSPGIQ, PGSGQNQ, QGSGQPQ, SGSMQNN, SGSGQNQ, VNHGQNQ, and WGSGQNQ.

39. An AAV capsid comprising an AAV capsid protein comprising substituting an amino acid sequence at amino acids 587-590 selected from AAAQ, AADQ, AAEQ, ACAF, ADAA, ADAF, ADAH, ADAL, ADAQ, ADRK, ADSQ, ADTQ, AEAC, AEAQ, AFAI, AGAQ, AHAD, AHAR, AHEQ, AKAQ, ALAD, ALAQ, ALNQ, AMDQ, AMGF, AMNQ, AMNY, AMWQ, ANAQ, ANAS, ANRQ, ANSD, APDQ, AQAD, AQAK, AQAM, AQAN, AQAP, AQAR, AQAW, AQCG, AQDD, AQDG, AQDL, AQDQ, AQEQ, AQFF, AQFG, AQFQ, AQGQ, AQIK, AQIQ, AQND, AQNN, AQNT, AQPG, AQWD, ARDQ, ARGK, ASWW, ATAQ, AVAQ, AYAQ, AYSQ, CQAG, DGAQ, DNNQ, DNYQ, DQAE, DQAQ, DQLD, DONS, DTAQ, DVAQ, DYAQ, ELAG, ELAQ, EMGQ, EQAF, EQAS, EQAY, EQGQ, EQNQ, ERAE, ESAQ, GQAQ, GQAY, GQPM, HMAQ, HQAD, HQAE, HQAR, HQDQ, IDAN, IEVQ, IHPQ, IMEQ, INAP, IQAN, IQAQ, KNHQ, KORQ, LETQ, LPAN, LQAQ, LTAP, MDAQ, MEAQ, MFAQ, MLAV, MLDQ, MQAD, MQAQ, MQAS, MQMQ, MQPD, MTAQ, MTPQ, NDAH, NDAS, NQAQ, PAAD, PDAQ, PLAQ, PQAD, PQAE, QAAQ, RQPQ, SDAN, TMAQ, TNAN, TNAQ, TQAQ, TQDG, TQGQ, TQQQ, VEAG, VEAQ, VLAQ, VQAQ, VQDA, VQEQ, WQAQ, WQQQ, YQAQ, YQNQ, and YWAQ.

40. The AAV capsid protein of claim 39 comprising substituting an amino acid sequence at amino acids 587-590 selected from ACAF, TNAQ, AMAF, SCAQ, SNAQ, FQAR, AQLC, AQAK, AQGT, AQEG, AQGQ, APDQ, TOAV, EQAS, MFAQ, VEAQ, EQAY, DVAQ, and MQMQ.

41. The AAV capsid protein of any of claims 1-40 wherein the AAV is AAV9.

42. The AAV capsid protein of any of claims 1-41 wherein the AAV is provided in SEQ ID NO: 1.

43. The AAV capsid protein of any one of claims 1-42, wherein 60 copies of the AAV capsid protein are assembled into the AAV capsid.

44. The AAV capsid protein of any of claims 1-43, wherein the AAV capsid protein is present in VP1, VP2, and VP3 of the AAV capsid.

45. The AAV capsid protein of any of claims 1-44, wherein the AAV capsid protein is characterized by at least one of an increased transduction enrichment when measured in a brain in a subject when delivered to the subject systemically.

46. The AAV capsid protein any of claims 1-45, wherein the AAV capsid protein further comprises an amino acid substitution comprising A589N or Q590P.

47. An AAV capsid comprising an AAV capsid protein of any of claims 1-46.

48. The AAV capsid of claim 47, wherein the AAV capsid is chimeric.

49. The AAV capsid of any of claims 47-48 that is isolated and purified.

50. The AAV capsid of any of claims 47-49 formulated as a pharmaceutical formulation for systemic administration to treat a disease or a condition of the brain, the pharmaceutical formulation further comprising a pharmaceutically acceptable carrier.

51. The AAV capsid protein of any of claims 1-46, wherein the capsid protein is expressed in the brain.

52. The AAV capsid protein of claim 51, wherein the brain comprises a cell-type selected from a neuron, an oligodendrocyte, an astrocyte, and a brain vascular cell.

53. The AAV capsid protein of any one of claims 36-37, wherein the brain comprises a tissue that is selected from brain, thalamus, cortex, putamen, lateral ventricles, medulla, the pons, the amygdala, the motor cortex, caudate, hypothalamus, striatum, ventral midbrain, neocortex, basal ganglia, hippocampus, thalamus, cerebrum, cerebellum, brain stem, and spinal cord.

54. A nucleic acid sequence encoding the peptide of any of claims 1-46.

55. A nucleic acid sequence encoding a peptide sequence as provided in Tables 1-3 or FIG. 1.

56. A recombinant vector comprising a nucleic acid encoding the AAV capsid protein of any one of claims 1-46.

57. A kit comprising:
a) a first vector comprising the recombinant vector of claim 56;
b) a second vector encoding a helper virus protein; and
c) a third vector comprising a therapeutic nucleic acid encoding a therapeutic gene expression product.

58. A method of treating a disease or condition in a subject comprising administering a therapeutically effective amount of a pharmaceutical formulation comprising the AAV capsid protein of any one of claims 1-46.

59. The method of claim 58, wherein the disease or the condition is a disease or a condition of a brain of the subject.

60. A method of manufacturing a recombinant AAV particle from the AAV capsid of any one of claims 1-46, the method comprising:
a. introducing into a cell a nucleic acid comprising:
i. a first nucleic acid sequence encoding a therapeutic gene expression product;
ii. a second nucleic acid sequence encoding a recombinant viral genome comprising a capsid (Cap) gene modified to express the AAV capsid of any one of claims 1-46; and
iii. a third nucleic acid sequence encoding an AAV helper virus genome; and
b. assembling the recombinant AAV particle, the recombinant AAV particle comprising the AAV capsid encapsidating the first nucleic acid.

61. The AAV capsid protein of any of claims 1-46 wherein the AAV capsid protein is characterized by an increased transduction enrichment when measured in a tissue in a subject when delivered to the subject systemically.

62. The AAV capsid protein of any of claims 1-46 wherein the tissue is brain tissue.

63. An AAV particle comprising an AAV capsid protein of any of claims 1-46 and a viral genome.

64. A peptide comprising an amino acid sequence selected from GNGTRLT, ENHTRSS, GNGTRPI, TNGTRPT, VNHVRSS, LNTIKPI, GNGTRIT, LNTTKLI, ENHVKSS, GNGTRPL, GNGTRPQ, ENNVRSS, and ENSVRSS.

65. A recombinant adeno-associated virus (rAAV) comprising a capsid comprising any one of AQGNGTRLTAQ, AQENHTRSSAQ, AQGNGTRPIAQ, AQTNGTRPTAQ, ACVNHVRSSAF, TNENHVRSRAQ, AQLNTIKPIAQ, AQGNGTRITAQ, AQLNTTKLIAQ, AQENHVKSSAQ, AQGNGTRPLAQ, AQGNGTRPQAQ, AQENNVRSSAQ, and AQENSVRSSAQ.


SEQUENCE LISTING (1273 sequences)

SEQ ID NO: 1 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 7 aa, synthetic construct
  VAR_SEQ 5: note = Xaa is selected from K and R
  VAR_SEQ 6: note = Xaa is selected from F, G, L, N, P, Q, R, S and V
  VAR_SEQ 7: note = Xaa is selected from C, D, E, F, L, M, N, P, Q, R, S, T and V
ENHVXXX

SEQ ID NO: 3 - skipped

SEQ ID NO: 4 - protein, 7 aa, synthetic construct
  VAR_SEQ 1: note = Xaa is selected from G, N, S and T
  VAR_SEQ 6: note = Xaa is selected from H, I, L, M, N, P, Q and T
  VAR_SEQ 7: note = Xaa is selected from I, L, M, N, Q, S and T
XNGTRXX

SEQ ID NO: 5 - skipped

SEQ ID NO: 6 - protein, 7 aa, synthetic construct
  VAR_SEQ 1: note = Xaa is selected from A, D, G, H, I, L, M, N, P, S, W and Y
XGSGQNQ

SEQ ID NO: 7 - protein, 7 aa, synthetic construct
  VAR_SEQ 5: note = Xaa is selected from F and Q
  VAR_SEQ 6: note = Xaa is selected from I, M and N
  VAR_SEQ 7: note = Xaa is selected from Q and V
NGSGXXX

SEQ ID NO: 8 - protein, 7 aa, synthetic construct
ENHVRSR

SEQ ID NO: 9 - protein, 7 aa, synthetic construct
ENHVKSS

SEQ ID NO: 10 - protein, 7 aa, synthetic construct
ENHVRST

SEQ ID NO: 11 - protein, 7 aa, synthetic construct
ENHVRNR

SEQ ID NO: 12 - protein, 7 aa, synthetic construct
ENHVRRS

SEQ ID NO: 13 - protein, 7 aa, synthetic construct
ENHVRFS

SEQ ID NO: 14 - protein, 7 aa, synthetic construct
ENHVRLS

SEQ ID NO: 15 - protein, 7 aa, synthetic construct
ENHTRSS

SEQ ID NO: 16 - protein, 7 aa, synthetic construct
VNHVRSS

SEQ ID NO: 17 - protein, 7 aa, synthetic construct
ENNVRSS

SEQ ID NO: 18 - protein, 7 aa, synthetic construct
ENSVRSS

SEQ ID NO: 19 - protein, 7 aa, synthetic construct
ENHTRSS

SEQ ID NO: 20 - protein, 7 aa, synthetic construct
ENHIRSS

SEQ ID NO: 21 - protein, 7 aa, synthetic construct
ENKVRSS

SEQ ID NO: 22 - protein, 7 aa, synthetic construct
ENRVRSS

SEQ ID NO: 23 - protein, 7 aa, synthetic construct
FNHVRSS

SEQ ID NO: 24 - protein, 7 aa, synthetic construct
NNHVRSS

SEQ ID NO: 25 - protein, 7 aa, synthetic construct
ENHTRSS

SEQ ID NO: 26 - protein, 7 aa, synthetic construct
GNGTRPI

SEQ ID NO: 27 - protein, 7 aa, synthetic construct
TNGTRPT

SEQ ID NO: 28 - protein, 7 aa, synthetic construct
VNHVRSS

SEQ ID NO: 29 - protein, 7 aa, synthetic construct
ENHVRSR

SEQ ID NO: 30 - protein, 7 aa, synthetic construct
GNGTRLT

SEQ ID NO: 31 - protein, 7 aa, synthetic construct
LNTIKPI

SEQ ID NO: 32 - protein, 7 aa, synthetic construct
GNGTRIT

SEQ ID NO: 33 - protein, 7 aa, synthetic construct
LNTTKLI

SEQ ID NO: 34 - protein, 7 aa, synthetic construct
ENHVKSS

SEQ ID NO: 35 - protein, 7 aa, synthetic construct
GNGTRPL

SEQ ID NO: 36 - protein, 7 aa, synthetic construct
GNGTRPQ

SEQ ID NO: 37 - protein, 7 aa, synthetic construct
ENNVRSS

SEQ ID NO: 38 - protein, 7 aa, synthetic construct
ENSVRSS

SEQ ID NO: 39 - protein, 7 aa, synthetic construct
GNGTRMT

SEQ ID NO: 40 - protein, 11 aa, synthetic construct
AQENHTRSSAQ

SEQ ID NO: 41 - protein, 11 aa, synthetic construct
AQGNGTRPIAQ

SEQ ID NO: 42 - protein, 11 aa, synthetic construct
AQTNGTRPTAQ

SEQ ID NO: 43 - protein, 11 aa, synthetic construct
ACVNHVRSSAF

SEQ ID NO: 44 - protein, 11 aa, synthetic construct
TNENHVRSRAQ

SEQ ID NO: 45 - protein, 11 aa, synthetic construct
AQGNGTRLTAQ

SEQ ID NO: 46 - protein, 11 aa, synthetic construct
AQLNTIKPIAQ

SEQ ID NO: 47 - protein, 11 aa, synthetic construct
AQGNGTRITAQ

SEQ ID NO: 48 - protein, 11 aa, synthetic construct
AQLNTTKLIAQ

SEQ ID NO: 49 - protein, 11 aa, synthetic construct
AQENHVKSSAQ

SEQ ID NO: 50 - protein, 11 aa, synthetic construct
AQGNGTRPLAQ

SEQ ID NO: 51 - protein, 11 aa, synthetic construct
AQGNGTRPQAQ

SEQ ID NO: 52 - protein, 11 aa, synthetic construct
AQENNVRSSAQ

SEQ ID NO: 53 - protein, 11 aa, synthetic construct
AQENSVRSSAQ

SEQ ID NO: 54 - protein, 11 aa, synthetic construct
AQGNGTRMTAQ

SEQ ID NO: 55 - protein, 7 aa, synthetic construct
AGSGQNQ

SEQ ID NO: 56 - protein, 7 aa, synthetic construct
DGSGQNQ

SEQ ID NO: 57 - protein, 7 aa, synthetic construct
GGSGQNQ

SEQ ID NO: 58 - protein, 7 aa, synthetic construct
HGSGQNQ

SEQ ID NO: 59 - protein, 7 aa, synthetic construct
IGSGQNQ

SEQ ID NO: 60 - protein, 7 aa, synthetic construct
LGSGQNQ

SEQ ID NO: 61 - protein, 7 aa, synthetic construct
MGSGQNQ

SEQ ID NO: 62 - protein, 7 aa, synthetic construct
NGFGQNQ

SEQ ID NO: 63 - protein, 7 aa, synthetic construct
NGIGQNQ

SEQ ID NO: 64 - protein, 7 aa, synthetic construct
NGNGQNQ

SEQ ID NO: 65 - protein, 7 aa, synthetic construct
NGSAQNQ

SEQ ID NO: 66 - protein, 7 aa, synthetic construct
NGSGFNQ

SEQ ID NO: 67 - protein, 7 aa, synthetic construct
NGSGQIQ

SEQ ID NO: 68 - protein, 7 aa, synthetic construct
NGSGQMQ

SEQ ID NO: 69 - protein, 7 aa, synthetic construct
NGSGQNQ

SEQ ID NO: 70 - protein, 7 aa, synthetic construct
NGSGQNV

SEQ ID NO: 71 - protein, 7 aa, synthetic construct
NGSLQNQ

SEQ ID NO: 72 - protein, 7 aa, synthetic construct
NGSNQNQ

SEQ ID NO: 73 - protein, 7 aa, synthetic construct
NGSTQNQ

SEQ ID NO: 74 - protein, 7 aa, synthetic construct
NLSGQNQ

SEQ ID NO: 75 - protein, 7 aa, synthetic construct
NSSGQNQ

SEQ ID NO: 76 - protein, 7 aa, synthetic construct
PGSGQNQ

SEQ ID NO: 77 - protein, 7 aa, synthetic construct
SGSGQNQ

SEQ ID NO: 78 - protein, 7 aa, synthetic construct
VGSGQNQ

SEQ ID NO: 79 - protein, 7 aa, synthetic construct
WGSGQNQ

SEQ ID NO: 80 - protein, 7 aa, synthetic construct
YGSGQNQ

SEQ ID NO: 81 - protein, 7 aa, synthetic construct
AGSGQNQ

SEQ ID NO: 82 - protein, 7 aa, synthetic construct
DGSGQNQ

SEQ ID NO: 83 - protein, 7 aa, synthetic construct
GGSGQNQ

SEQ ID NO: 84 - protein, 7 aa, synthetic construct
HGSGQNQ

SEQ ID NO: 85 - protein, 7 aa, synthetic construct
IGSGQNQ

SEQ ID NO: 86 - protein, 7 aa, synthetic construct
LGSGQNQ

SEQ ID NO: 87 - protein, 7 aa, synthetic construct
MGSGQNQ

SEQ ID NO: 88 - protein, 7 aa, synthetic construct
NGFGQNQ

SEQ ID NO: 89 - protein, 7 aa, synthetic construct
NGIGQNQ

SEQ ID NO: 90 - protein, 7 aa, synthetic construct
NGNGQNQ

SEQ ID NO: 91 - protein, 7 aa, synthetic construct
NGSAQNQ

SEQ ID NO: 92 - protein, 7 aa, synthetic construct
NGSGFNQ

SEQ ID NO: 93 - protein, 7 aa, synthetic construct
NGSGQIQ

SEQ ID NO: 94 - protein, 7 aa, synthetic construct
NGSGQMQ

SEQ ID NO: 95 - protein, 7 aa, synthetic construct
NGSGQNQ

SEQ ID NO: 96 - protein, 7 aa, synthetic construct
NGSGQNV

SEQ ID NO: 97 - protein, 7 aa, synthetic construct
NGSLQNQ

SEQ ID NO: 98 - protein, 7 aa, synthetic construct
NGSNQNQ

SEQ ID NO: 99 - protein, 7 aa, synthetic construct
NGSTQNQ

SEQ ID NO: 100 - protein, 7 aa, synthetic construct
NLSGQNQ

SEQ ID NO: 101 - protein, 7 aa, synthetic construct
NSSGQNQ

SEQ ID NO: 102 - protein, 7 aa, synthetic construct
PGSGQNQ

SEQ ID NO: 103 - protein, 7 aa, synthetic construct
SGSGQNQ

SEQ ID NO: 104 - protein, 7 aa, synthetic construct
VGSGQNQ

SEQ ID NO: 105 - protein, 7 aa, synthetic construct
WGSGQNQ

SEQ ID NO: 106 - protein, 7 aa, synthetic construct
YGSGQNQ

SEQ ID NO: 107 - protein, 7 aa, synthetic construct
DGSGQNQ

SEQ ID NO: 108 - protein, 7 aa, synthetic construct
FGSGQNQ

SEQ ID NO: 109 - protein, 7 aa, synthetic construct
GGSGQNQ

SEQ ID NO: 110 - protein, 7 aa, synthetic construct
IGSGQNQ

SEQ ID NO: 111 - protein, 7 aa, synthetic construct
MGSGQNQ

SEQ ID NO: 112 - protein, 7 aa, synthetic construct
NGSAQNQ

SEQ ID NO: 113 - protein, 7 aa, synthetic construct
NGSGQFG

SEQ ID NO: 114 - protein, 7 aa, synthetic construct
NGSGQLQ

SEQ ID NO: 115 - protein, 7 aa, synthetic construct
NGSGQNQ

SEQ ID NO: 116 - protein, 7 aa, synthetic construct
NGSGQNV

SEQ ID NO: 117 - protein, 7 aa, synthetic construct
NGSLQNQ

SEQ ID NO: 118 - protein, 7 aa, synthetic construct
NGSMQNQ

SEQ ID NO: 119 - protein, 7 aa, synthetic construct
NGSSQNQ

SEQ ID NO: 120 - protein, 7 aa, synthetic construct
NGSTQNQ

SEQ ID NO: 121 - protein, 7 aa, synthetic construct
PGSGQNQ

SEQ ID NO: 122 - protein, 7 aa, synthetic construct
QGSGQNQ

SEQ ID NO: 123 - protein, 7 aa, synthetic construct
SGSGQNQ

SEQ ID NO: 124 - protein, 7 aa, synthetic construct
TGSGQNQ

SEQ ID NO: 125 - protein, 7 aa, synthetic construct
VGSGQNQ

SEQ ID NO: 126 - protein, 7 aa, synthetic construct
WGSGQNQ

SEQ ID NO: 127 - protein, 7 aa, synthetic construct
AGSGQNQ

SEQ ID NO: 128 - protein, 7 aa, synthetic construct
APSGQNQ

SEQ ID NO: 129 - protein, 7 aa, synthetic construct
GGSGQNQ

SEQ ID NO: 130 - protein, 7 aa, synthetic construct
HGSGQNQ

SEQ ID NO: 131 - protein, 7 aa, synthetic construct
LGSGQNQ

SEQ ID NO: 132 - protein, 7 aa, synthetic construct
NGNGQNQ

SEQ ID NO: 133 - protein, 7 aa, synthetic construct
NVSGQNQ

SEQ ID NO: 134 - protein, 7 aa, synthetic construct
NGSGQNQ

SEQ ID NO: 135 - protein, 7 aa, synthetic construct
NGFGQNQ

SEQ ID NO: 136 - protein, 7 aa, synthetic construct
NGSGQIQ

SEQ ID NO: 137 - protein, 7 aa, synthetic construct
NSSGQNQ

SEQ ID NO: 138 - protein, 7 aa, synthetic construct
NGSPGIQ

SEQ ID NO: 139 - protein, 7 aa, synthetic construct
PGSGQNQ

SEQ ID NO: 140 - protein, 7 aa, synthetic construct
QGSGQPQ

SEQ ID NO: 141 - protein, 7 aa, synthetic construct
SGSMQNN

SEQ ID NO: 142 - protein, 7 aa, synthetic construct
SGSGQNQ

SEQ ID NO: 143 - protein, 7 aa, synthetic construct
VNHGQNQ

SEQ ID NO: 144 - protein, 7 aa, synthetic construct
WGSGQNQ

SEQ ID NO: 145 - protein, 4 aa, synthetic construct
AAAQ

SEQ ID NO: 146 - protein, 4 aa, synthetic construct
AADQ

SEQ ID NO: 147 - protein, 4 aa, synthetic construct
AAEQ

SEQ ID NO: 148 - protein, 4 aa, synthetic construct
ACAF

SEQ ID NO: 149 - protein, 4 aa, synthetic construct
ADAA

SEQ ID NO: 150 - protein, 4 aa, synthetic construct
ADAF

SEQ ID NO: 151 - protein, 4 aa, synthetic construct
ADAH

SEQ ID NO: 152 - protein, 4 aa, synthetic construct
ADAL

SEQ ID NO: 153 - protein, 4 aa, synthetic construct
ADAQ

SEQ ID NO: 154 - protein, 4 aa, synthetic construct
ADRK

SEQ ID NO: 155 - protein, 4 aa, synthetic construct
ADSQ

SEQ ID NO: 156 - protein, 4 aa, synthetic construct
ADTQ

SEQ ID NO: 157 - protein, 4 aa, synthetic construct
AEAC

SEQ ID NO: 158 - protein, 4 aa, synthetic construct
AEAQ

SEQ ID NO: 159 - protein, 4 aa, synthetic construct
AFAI

SEQ ID NO: 160 - protein, 4 aa, synthetic construct
AGAQ

SEQ ID NO: 161 - protein, 4 aa, synthetic construct
AHAD

SEQ ID NO: 162 - protein, 4 aa, synthetic construct
AHAR

SEQ ID NO: 163 - protein, 4 aa, synthetic construct
AHEQ

SEQ ID NO: 164 - protein, 4 aa, synthetic construct
AKAQ

SEQ ID NO: 165 - protein, 4 aa, synthetic construct
ALAD

SEQ ID NO: 166 - protein, 4 aa, synthetic construct
ALAQ

SEQ ID NO: 167 - protein, 4 aa, synthetic construct
ALNQ

SEQ ID NO: 168 - protein, 4 aa, synthetic construct
AMDQ

SEQ ID NO: 169 - protein, 4 aa, synthetic construct
AMGF

SEQ ID NO: 170 - protein, 4 aa, synthetic construct
AMNQ

SEQ ID NO: 171 - protein, 4 aa, synthetic construct
AMNY

SEQ ID NO: 172 - protein, 4 aa, synthetic construct
AMWQ

SEQ ID NO: 173 - protein, 4 aa, synthetic construct
ANAQ

SEQ ID NO: 174 - protein, 4 aa, synthetic construct
ANAS

SEQ ID NO: 175 - protein, 4 aa, synthetic construct
ANRQ

SEQ ID NO: 176 - protein, 4 aa, synthetic construct
ANSD

SEQ ID NO: 177 - protein, 4 aa, synthetic construct
APDQ

SEQ ID NO: 178 - protein, 4 aa, synthetic construct
AQAD

SEQ ID NO: 179 - protein, 4 aa, synthetic construct
AQAK

SEQ ID NO: 180 - protein, 4 aa, synthetic construct
AQAM

SEQ ID NO: 181 - protein, 4 aa, synthetic construct
AQAN

SEQ ID NO: 182 - protein, 4 aa, synthetic construct
AQAP

SEQ ID NO: 183 - protein, 4 aa, synthetic construct
AQAR

SEQ ID NO: 184 - protein, 4 aa, synthetic construct
AQAW

SEQ ID NO: 185 - protein, 4 aa, synthetic construct
AQCG

SEQ ID NO: 186 - protein, 4 aa, synthetic construct
AQDD

SEQ ID NO: 187 - protein, 4 aa, synthetic construct
AQDG

SEQ ID NO: 188 - protein, 4 aa, synthetic construct
AQDL

SEQ ID NO: 189 - protein, 4 aa, synthetic construct
AQDQ

SEQ ID NO: 190 - protein, 4 aa, synthetic construct
AQEQ

SEQ ID NO: 191 - protein, 4 aa, synthetic construct
AQFF

SEQ ID NO: 192 - protein, 4 aa, synthetic construct
AQFG

SEQ ID NO: 193 - protein, 4 aa, synthetic construct
AQFQ

SEQ ID NO: 194 - protein, 4 aa, synthetic construct
AQGQ

SEQ ID NO: 195 - protein, 4 aa, synthetic construct
AQIK

SEQ ID NO: 196 - protein, 4 aa, synthetic construct
AQIQ

SEQ ID NO: 197 - protein, 4 aa, synthetic construct
AQND

SEQ ID NO: 198 - protein, 4 aa, synthetic construct
AQNN

SEQ ID NO: 199 - protein, 4 aa, synthetic construct
AQNT

SEQ ID NO: 200 - protein, 4 aa, synthetic construct
AQPG

SEQ ID NO: 201 - protein, 4 aa, synthetic construct
AQWD

SEQ ID NO: 202 - protein, 4 aa, synthetic construct
ARDQ

SEQ ID NO: 203 - protein, 4 aa, synthetic construct
ARGK

SEQ ID NO: 204 - protein, 4 aa, synthetic construct
ASWW

SEQ ID NO: 205 - protein, 4 aa, synthetic construct
ATAQ

SEQ ID NO: 206 - protein, 4 aa, synthetic construct
AVAQ

SEQ ID NO: 207 - protein, 4 aa, synthetic construct
AYAQ

SEQ ID NO: 208 - protein, 4 aa, synthetic construct
AYSQ

SEQ ID NO: 209 - protein, 4 aa, synthetic construct
CQAG

SEQ ID NO: 210 - protein, 4 aa, synthetic construct
DGAQ

SEQ ID NO: 211 - protein, 4 aa, synthetic construct
DNNQ

SEQ ID NO: 212 - protein, 4 aa, synthetic construct
DNYQ

SEQ ID NO: 213 - protein, 4 aa, synthetic construct
DQAE

SEQ ID NO: 214 - protein, 4 aa, synthetic construct
DQAQ

SEQ ID NO: 215 - protein, 4 aa, synthetic construct
DQLD

SEQ ID NO: 216 - protein, 4 aa, synthetic construct
DQNS

SEQ ID NO: 217 - protein, 4 aa, synthetic construct
DTAQ

SEQ ID NO: 218 - protein, 4 aa, synthetic construct
DVAQ

SEQ ID NO: 219 - protein, 4 aa, synthetic construct
DYAQ

SEQ ID NO: 220 - protein, 4 aa, synthetic construct
ELAG

SEQ ID NO: 221 - protein, 4 aa, synthetic construct
ELAQ

SEQ ID NO: 222 - protein, 4 aa, synthetic construct
EMGQ

SEQ ID NO: 223 - protein, 4 aa, synthetic construct
EQAF

SEQ ID NO: 224 - protein, 4 aa, synthetic construct
EQAS

SEQ ID NO: 225 - protein, 4 aa, synthetic construct
EQAY

SEQ ID NO: 226 - protein, 4 aa, synthetic construct
EQGQ

SEQ ID NO: 227 - protein, 4 aa, synthetic construct
EQNQ

SEQ ID NO: 228 - protein, 4 aa, synthetic construct
ERAE

SEQ ID NO: 229 - protein, 4 aa, synthetic construct
ESAQ

SEQ ID NO: 230 - protein, 4 aa, synthetic construct
GQAQ

SEQ ID NO: 231 - protein, 4 aa, synthetic construct
GQAY

SEQ ID NO: 232 - protein, 4 aa, synthetic construct
GQPM

SEQ ID NO: 233 - protein, 4 aa, synthetic construct
HMAQ

SEQ ID NO: 234 - protein, 4 aa, synthetic construct
HQAD

SEQ ID NO: 235 - protein, 4 aa, synthetic construct
HQAE

SEQ ID NO: 236 - protein, 4 aa, synthetic construct
HQAR

SEQ ID NO: 237 - protein, 4 aa, synthetic construct
HQDQ

SEQ ID NO: 238 - protein, 4 aa, synthetic construct
IDAN

SEQ ID NO: 239 - protein, 4 aa, synthetic construct
IEVQ

SEQ ID NO: 240 - protein, 4 aa, synthetic construct
IHPQ

SEQ ID NO: 241 - protein, 4 aa, synthetic construct
IMEQ

SEQ ID NO: 242 - protein, 4 aa, synthetic construct
INAP

SEQ ID NO: 243 - protein, 4 aa, synthetic construct
IQAN

SEQ ID NO: 244 - protein, 4 aa, synthetic construct
IQAQ

SEQ ID NO: 245 - protein, 4 aa, synthetic construct
KNHQ

SEQ ID NO: 246 - protein, 4 aa, synthetic construct
KQRQ

SEQ ID NO: 247 - protein, 4 aa, synthetic construct
LETQ

SEQ ID NO: 248 - protein, 4 aa, synthetic construct
LPAN

SEQ ID NO: 249 - protein, 4 aa, synthetic construct
LQAQ

SEQ ID NO: 250 - protein, 4 aa, synthetic construct
LTAP

SEQ ID NO: 251 - protein, 4 aa, synthetic construct
MDAQ

SEQ ID NO: 252 - protein, 4 aa, synthetic construct
MEAQ

SEQ ID NO: 253 - protein, 4 aa, synthetic construct
MFAQ

SEQ ID NO: 254 - protein, 4 aa, synthetic construct
MLAV

SEQ ID NO: 255 - protein, 4 aa, synthetic construct
MLDQ

SEQ ID NO: 256 - protein, 4 aa, synthetic construct
MQAD

SEQ ID NO: 257 - protein, 4 aa, synthetic construct
MQAQ

SEQ ID NO: 258 - protein, 4 aa, synthetic construct
MQAS

SEQ ID NO: 259 - protein, 4 aa, synthetic construct
MQMQ

SEQ ID NO: 260 - protein, 4 aa, synthetic construct
MQPD

SEQ ID NO: 261 - protein, 4 aa, synthetic construct
MTAQ

SEQ ID NO: 262 - protein, 4 aa, synthetic construct
MTPQ

SEQ ID NO: 263 - protein, 4 aa, synthetic construct
NDAH

SEQ ID NO: 264 - protein, 4 aa, synthetic construct
NDAS

SEQ ID NO: 265 - protein, 4 aa, synthetic construct
NQAQ

SEQ ID NO: 266 - protein, 4 aa, synthetic construct
PAAD

SEQ ID NO: 267 - protein, 4 aa, synthetic construct
PDAQ

SEQ ID NO: 268 - protein, 4 aa, synthetic construct
PLAQ

SEQ ID NO: 269 - protein, 4 aa, synthetic construct
PQAD

SEQ ID NO: 270 - protein, 4 aa, synthetic construct
PQAE

SEQ ID NO: 271 - protein, 4 aa, synthetic construct
QAAQ

SEQ ID NO: 272 - protein, 4 aa, synthetic construct
RQPQ

SEQ ID NO: 273 - protein, 4 aa, synthetic construct
SDAN

SEQ ID NO: 274 - protein, 4 aa, synthetic construct
TMAQ

SEQ ID NO: 275 - protein, 4 aa, synthetic construct
TNAN

SEQ ID NO: 276 - protein, 4 aa, synthetic construct
TNAQ

SEQ ID NO: 277 - protein, 4 aa, synthetic construct
TQAQ

SEQ ID NO: 278 - protein, 4 aa, synthetic construct
TQDG

SEQ ID NO: 279 - protein, 4 aa, synthetic construct
TQGQ

SEQ ID NO: 280 - protein, 4 aa, synthetic construct
TQQQ

SEQ ID NO: 281 - protein, 4 aa, synthetic construct
VEAG

SEQ ID NO: 282 - protein, 4 aa, synthetic construct
VEAQ

SEQ ID NO: 283 - protein, 4 aa, synthetic construct
VLAQ

SEQ ID NO: 284 - protein, 4 aa, synthetic construct
VQAQ

SEQ ID NO: 285 - protein, 4 aa, synthetic construct
VQDA

SEQ ID NO: 286 - protein, 4 aa, synthetic construct
VQEQ

SEQ ID NO: 287 - protein, 4 aa, synthetic construct
WQAQ

SEQ ID NO: 288 - protein, 4 aa, synthetic construct
WQQQ

SEQ ID NO: 289 - protein, 4 aa, synthetic construct
YQAQ

SEQ ID NO: 290 - protein, 4 aa, synthetic construct
YQNQ

SEQ ID NO: 291 - protein, 4 aa, synthetic construct
YWAQ

SEQ ID NO: 292 - protein, 4 aa, synthetic construct
ACAF

SEQ ID NO: 293 - protein, 4 aa, synthetic construct
TNAQ

SEQ ID NO: 294 - protein, 4 aa, synthetic construct
AMAF

SEQ ID NO: 295 - protein, 4 aa, synthetic construct
SCAQ

SEQ ID NO: 296 - protein, 4 aa, synthetic construct
SNAQ

SEQ ID NO: 297 - protein, 4 aa, synthetic construct
FQAR

SEQ ID NO: 298 - protein, 4 aa, synthetic construct
AQLC

SEQ ID NO: 299 - protein, 4 aa, synthetic construct
AQAK

SEQ ID NO: 300 - protein, 4 aa, synthetic construct
AQGT

SEQ ID NO: 301 - protein, 4 aa, synthetic construct
AQEG

SEQ ID NO: 302 - protein, 4 aa, synthetic construct
AQGQ

SEQ ID NO: 303 - protein, 4 aa, synthetic construct
APDQ

SEQ ID NO: 304 - protein, 4 aa, synthetic construct
TQAV

SEQ ID NO: 305 - protein, 4 aa, synthetic construct
EQAS

SEQ ID NO: 306 - protein, 4 aa, synthetic construct
MFAQ

SEQ ID NO: 307 - protein, 4 aa, synthetic construct
VEAQ

SEQ ID NO: 308 - protein, 4 aa, synthetic construct
EQAY

SEQ ID NO: 309 - protein, 4 aa, synthetic construct
DVAQ

SEQ ID NO: 310 - protein, 4 aa, synthetic construct
MQMQ

SEQ ID NO: 311 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGNGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQENHTRSSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 312 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINVSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNGTRPIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 313 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIPGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTNGTRPTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 314 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSACVNHVRSSAFAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 315 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSTNENHVRSRAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 316 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIPGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNGTRLTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 317 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIHGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLNTIKPIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 318 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIGGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNGTRITAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 319 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTIGGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLNTTKLIAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 320 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGFGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQENHVKSSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 321 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 322 - protein, 11 aa, synthetic construct
AQENHTRSSAQ

SEQ ID NO: 323 - protein, 11 aa, synthetic construct
AQGNGTRPIAQ

SEQ ID NO: 324 - protein, 11 aa, synthetic construct
AQTNGTRPTAQ

SEQ ID NO: 325 - protein, 11 aa, synthetic construct
ACVNHVRSSAF

SEQ ID NO: 326 - protein, 11 aa, synthetic construct
TNENHVRSRAQ

SEQ ID NO: 327 - protein, 11 aa, synthetic construct
AQGNGTRLTAQ

SEQ ID NO: 328 - protein, 11 aa, synthetic construct
AQLNTIKPIAQ

SEQ ID NO: 329 - protein, 11 aa, synthetic construct
AQGNGTRITAQ

SEQ ID NO: 330 - protein, 11 aa, synthetic construct
AQLNTTKLIAQ

SEQ ID NO: 331 - protein, 11 aa, synthetic construct
AQENHVKSSAQ

SEQ ID NO: 332 - protein, 11 aa, synthetic construct
AQGNGTRPLAQ

SEQ ID NO: 333 - protein, 11 aa, synthetic construct
AQGNGTRPQAQ

SEQ ID NO: 334 - protein, 11 aa, synthetic construct
AQENNVRSSAQ

SEQ ID NO: 335 - protein, 11 aa, synthetic construct
AQENSVRSSAQ

SEQ ID NO: 336 - protein, 11 aa, synthetic construct
AQGNGTRMTAQ

SEQ ID NO: 337 - protein, 11 aa, synthetic construct
AQENSVRSSAQ

SEQ ID NO: 338 - protein, 11 aa, synthetic construct
AQENSVRSSAQ

SEQ ID NO: 339 - protein, 11 aa, synthetic construct
AQENHTRSSAQ

SEQ ID NO: 340 - protein, 11 aa, synthetic construct
AQENHIRSSAQ

SEQ ID NO: 341 - protein, 11 aa, synthetic construct
AQENKVRSSAQ

SEQ ID NO: 342 - protein, 11 aa, synthetic construct
AQENHIRSSAQ

SEQ ID NO: 343 - protein, 11 aa, synthetic construct
AQGNGTRPTAQ

SEQ ID NO: 344 - protein, 11 aa, synthetic construct
AQGNGTRPTAQ

SEQ ID NO: 345 - protein, 11 aa, synthetic construct
MQENRVRSSMQ

SEQ ID NO: 346 - protein, 11 aa, synthetic construct
AQGNGTRPSAQ

SEQ ID NO: 347 - protein, 11 aa, synthetic construct
AQGNGTRQTAQ

SEQ ID NO: 348 - protein, 11 aa, synthetic construct
AQGNGTRNTAQ

SEQ ID NO: 349 - protein, 11 aa, synthetic construct
AQLNNTKPIAQ

SEQ ID NO: 350 - protein, 11 aa, synthetic construct
AQGNGTRPMAQ

SEQ ID NO: 351 - protein, 11 aa, synthetic construct
AQGNGTRPNAQ

SEQ ID NO: 352 - protein, 11 aa, synthetic construct
AQENHVRSTAQ

SEQ ID NO: 353 - protein, 11 aa, synthetic construct
AQGNGTRITAQ

SEQ ID NO: 354 - protein, 11 aa, synthetic construct
AQENNVRSSAQ

SEQ ID NO: 355 - protein, 11 aa, synthetic construct
AQGNGTRTTAQ

SEQ ID NO: 356 - protein, 11 aa, synthetic construct
AQGNGTRPLAQ

SEQ ID NO: 357 - protein, 11 aa, synthetic construct
AQGNGTRTTAQ

SEQ ID NO: 358 - protein, 11 aa, synthetic construct
ACFNHVRSSAQ

SEQ ID NO: 359 - protein, 11 aa, synthetic construct
AQGNGTRPQAQ

SEQ ID NO: 360 - protein, 11 aa, synthetic construct
AQGNGTRPQAQ

SEQ ID NO: 361 - protein, 11 aa, synthetic construct
AQGNGTRPTAQ

SEQ ID NO: 362 - protein, 11 aa, synthetic construct
AQGNGTRPMAQ

SEQ ID NO: 363 - protein, 11 aa, synthetic construct
AQENRVRSSAQ

SEQ ID NO: 364 - protein, 11 aa, synthetic construct
AQLNNTKPIAQ

SEQ ID NO: 365 - protein, 11 aa, synthetic construct
AQQLRDKSSAQ

SEQ ID NO: 366 - protein, 11 aa, synthetic construct
AQGNNTRPTAQ

SEQ ID NO: 367 - protein, 11 aa, synthetic construct
AQGNGTRPTAQ

SEQ ID NO: 368 - protein, 11 aa, synthetic construct
AQGNGNRPTAQ

SEQ ID NO: 369 - protein, 11 aa, synthetic construct
AQNNHVRSSAQ

SEQ ID NO: 370 - protein, 11 aa, synthetic construct
AQLNTTKPIAQ

SEQ ID NO: 371 - protein, 11 aa, synthetic construct
AQNNGTRPTAQ

SEQ ID NO: 372 - protein, 11 aa, synthetic construct
AQGNGTRPNAQ

SEQ ID NO: 373 - protein, 11 aa, synthetic construct
AQGNGTKPTAQ

SEQ ID NO: 374 - protein, 11 aa, synthetic construct
AQGNGTKPTAQ

SEQ ID NO: 375 - protein, 11 aa, synthetic construct
AQENKVRSSAQ

SEQ ID NO: 376 - protein, 11 aa, synthetic construct
AQGNGTRPMAQ

SEQ ID NO: 377 - protein, 11 aa, synthetic construct
AQGNGTRPTAQ

SEQ ID NO: 378 - protein, 11 aa, synthetic construct
AQGNGTRPTAQ

SEQ ID NO: 379 - protein, 11 aa, synthetic construct
AQGNGTRTTAQ

SEQ ID NO: 380 - protein, 11 aa, synthetic construct
AQTLKSPAKAQ

SEQ ID NO: 381 - protein, 11 aa, synthetic construct
AQGNGTRPLAQ

SEQ ID NO: 382 - protein, 11 aa, synthetic construct
AQGNGTRNTAQ

SEQ ID NO: 383 - protein, 11 aa, synthetic construct
AQGNGTRPTAQ

SEQ ID NO: 384 - protein, 11 aa, synthetic construct
AQGNGTRPTAQ

SEQ ID NO: 385 - protein, 11 aa, synthetic construct
AQENIVRSSAQ

SEQ ID NO: 386 - protein, 11 aa, synthetic construct
AQENHVRNRTQ

SEQ ID NO: 387 - protein, 11 aa, synthetic construct
AMENTVRSSAF

SEQ ID NO: 388 - protein, 11 aa, synthetic construct
AQLLRDKSTAQ

SEQ ID NO: 389 - protein, 11 aa, synthetic construct
AQGNGTRPTAQ

SEQ ID NO: 390 - protein, 11 aa, synthetic construct
AQGNGTRPTAQ

SEQ ID NO: 391 - protein, 11 aa, synthetic construct
DQENHVRLNAQ

SEQ ID NO: 392 - protein, 11 aa, synthetic construct
SCENHVRFSAQ

SEQ ID NO: 393 - protein, 11 aa, synthetic construct
AQGNGTRPQAQ

SEQ ID NO: 394 - protein, 11 aa, synthetic construct
AQGNGTRHTAQ

SEQ ID NO: 395 - protein, 11 aa, synthetic construct
AQENTVRSSAQ

SEQ ID NO: 396 - protein, 11 aa, synthetic construct
SNENHVRRSAQ

SEQ ID NO: 397 - protein, 11 aa, synthetic construct
AQENMVRSSAQ

SEQ ID NO: 398 - protein, 11 aa, synthetic construct
AQLNTTKPIAQ

SEQ ID NO: 399 - protein, 11 aa, synthetic construct
AQGNGTRPTAQ

SEQ ID NO: 400 - protein, 11 aa, synthetic construct
AQGNGTRPTAQ

SEQ ID NO: 401 - protein, 11 aa, synthetic construct
AQLNTTKPIAQ

SEQ ID NO: 402 - protein, 11 aa, synthetic construct
AQLNTTKPIAQ

SEQ ID NO: 403 - protein, 11 aa, synthetic construct
AQLNTTKPIAQ

SEQ ID NO: 404 - protein, 11 aa, synthetic construct
AQGNTTRPTAQ

SEQ ID NO: 405 - protein, 11 aa, synthetic construct
AQGNGTRPTAQ

SEQ ID NO: 406 - protein, 11 aa, synthetic construct
AQSNGTRPTAQ

SEQ ID NO: 407 - protein, 11 aa, synthetic construct
AQGNNTRPTAQ

SEQ ID NO: 408 - protein, 11 aa, synthetic construct
AQLNTTKSIAQ

SEQ ID NO: 409 - protein, 11 aa, synthetic construct
AQGNGTRTTAQ

SEQ ID NO: 410 - protein, 11 aa, synthetic construct
FQENFVRSSAR

SEQ ID NO: 411 - protein, 11 aa, synthetic construct
AQENHVRLSLC

SEQ ID NO: 412 - protein, 11 aa, synthetic construct
AQENRVRSSAQ

SEQ ID NO: 413 - protein, 11 aa, synthetic construct
AQGNGTKPTAQ

SEQ ID NO: 414 - protein, 11 aa, synthetic construct
AQGNGTRPTAQ

SEQ ID NO: 415 - protein, 11 aa, synthetic construct
AQGNGTRPTAQ

SEQ ID NO: 416 - protein, 11 aa, synthetic construct
AQGNGTRPTAQ

SEQ ID NO: 417 - protein, 11 aa, synthetic construct
AQENSVRSSAQ

SEQ ID NO: 418 - protein, 11 aa, synthetic construct
AQGNGTRNTAQ

SEQ ID NO: 419 - protein, 11 aa, synthetic construct
AQENHIRSSAQ

SEQ ID NO: 420 - protein, 11 aa, synthetic construct
AQLNTTKTIAQ

SEQ ID NO: 421 - protein, 11 aa, synthetic construct
AQGNGTRPTAQ

SEQ ID NO: 422 - protein, 11 aa, synthetic construct
AQGNGTRPTAQ

SEQ ID NO: 423 - protein, 11 aa, synthetic construct
AQGNGTRPTAQ

SEQ ID NO: 424 - protein, 11 aa, synthetic construct
AQENNVRSSAQ

SEQ ID NO: 425 - protein, 11 aa, synthetic construct
AQETHVRSSAQ

SEQ ID NO: 426 - protein, 11 aa, synthetic construct
AQGNGTRPLAQ

SEQ ID NO: 427 - protein, 11 aa, synthetic construct
AQENRVRSSAQ

SEQ ID NO: 428 - protein, 11 aa, synthetic construct
AQGNGTRPTAQ

SEQ ID NO: 429 - protein, 11 aa, synthetic construct
AQTNTTKPIAQ

SEQ ID NO: 430 - protein, 11 aa, synthetic construct
AQENHVRSPAQ

SEQ ID NO: 431 - protein, 11 aa, synthetic construct
AQSNHVRSSAQ

SEQ ID NO: 432 - protein, 11 aa, synthetic construct
AQGNVVRSSAK

SEQ ID NO: 433 - protein, 11 aa, synthetic construct
AQENNVRSSAQ

SEQ ID NO: 434 - protein, 11 aa, synthetic construct
AQGNGTRNTAQ

SEQ ID NO: 435 - protein, 11 aa, synthetic construct
AQGNGTRPLAQ

SEQ ID NO: 436 - protein, 11 aa, synthetic construct
AQENKVRSSAQ

SEQ ID NO: 437 - protein, 11 aa, synthetic construct
MFENTVRSSAQ

SEQ ID NO: 438 - protein, 11 aa, synthetic construct
AQLNTTKNIAQ

SEQ ID NO: 439 - protein, 11 aa, synthetic construct
AQGNGTRPLAQ

SEQ ID NO: 440 - protein, 11 aa, synthetic construct
AQGNGTRPTAQ

SEQ ID NO: 441 - protein, 11 aa, synthetic construct
AQGNGTRPTAQ

SEQ ID NO: 442 - protein, 11 aa, synthetic construct
AQGNGTRPTAQ

SEQ ID NO: 443 - protein, 11 aa, synthetic construct
AQGNGTRPTAQ

SEQ ID NO: 444 - protein, 11 aa, synthetic construct
AQGNGTRPTAQ

SEQ ID NO: 445 - protein, 11 aa, synthetic construct
AQGNGTRPTAQ

SEQ ID NO: 446 - protein, 11 aa, synthetic construct
AQGNGTRPTAQ

SEQ ID NO: 447 - protein, 11 aa, synthetic construct
AQGNGIRPTAQ

SEQ ID NO: 448 - protein, 11 aa, synthetic construct
AQENRVRSSAQ

SEQ ID NO: 449 - protein, 11 aa, synthetic construct
AQENRVRSSAQ

SEQ ID NO: 450 - protein, 11 aa, synthetic construct
AQGNGTRLTAQ

SEQ ID NO: 451 - protein, 11 aa, synthetic construct
AQGNGTRLTAQ

SEQ ID NO: 452 - protein, 11 aa, synthetic construct
AQGNGTRLTAQ

SEQ ID NO: 453 - protein, 11 aa, synthetic construct
AQGNGTRTTAQ

SEQ ID NO: 454 - protein, 11 aa, synthetic construct
AQGNGTRPTAQ

SEQ ID NO: 455 - protein, 11 aa, synthetic construct
AQGNGTRNTAQ

SEQ ID NO: 456 - protein, 11 aa, synthetic construct
AQLNTTKNIAQ

SEQ ID NO: 457 - protein, 11 aa, synthetic construct
AQGNGTRITAQ

SEQ ID NO: 458 - protein, 11 aa, synthetic construct
AQENIVRSSAQ

SEQ ID NO: 459 - protein, 11 aa, synthetic construct
AQGNGTRPTAQ

SEQ ID NO: 460 - protein, 11 aa, synthetic construct
AQGNGTRPTAQ

SEQ ID NO: 461 - protein, 11 aa, synthetic construct
AQRNHVRSSGT

SEQ ID NO: 462 - protein, 11 aa, synthetic construct
AQLNRDKSSAQ

SEQ ID NO: 463 - protein, 11 aa, synthetic construct
AQGNGTRLTAQ

SEQ ID NO: 464 - protein, 11 aa, synthetic construct
AQGNGTRNTAQ

SEQ ID NO: 465 - protein, 11 aa, synthetic construct
AQGNGTRTTAQ

SEQ ID NO: 466 - protein, 11 aa, synthetic construct
AQGNGTRTTAQ

SEQ ID NO: 467 - protein, 11 aa, synthetic construct
AQENHVRNSAQ

SEQ ID NO: 468 - protein, 11 aa, synthetic construct
YQENHVRFQAQ

SEQ ID NO: 469 - protein, 11 aa, synthetic construct
AQENHVRSTEG

SEQ ID NO: 470 - protein, 11 aa, synthetic construct
AQLNTTKMIAQ

SEQ ID NO: 471 - protein, 11 aa, synthetic construct
PDENHVRSQAQ

SEQ ID NO: 472 - protein, 11 aa, synthetic construct
AQGNGTRPTAQ

SEQ ID NO: 473 - protein, 11 aa, synthetic construct
TQENVVRSSAV

SEQ ID NO: 474 - protein, 11 aa, synthetic construct
AQENKVRSSAQ

SEQ ID NO: 475 - protein, 11 aa, synthetic construct
AQGNGTRMTAQ

SEQ ID NO: 476 - protein, 11 aa, synthetic construct
EQENHVRSTAS

SEQ ID NO: 477 - protein, 11 aa, synthetic construct
VEENRVRSSAQ

SEQ ID NO: 478 - protein, 11 aa, synthetic construct
AQLNQTKPIAQ

SEQ ID NO: 479 - protein, 11 aa, synthetic construct
AQGNGTRHTAQ

SEQ ID NO: 480 - protein, 11 aa, synthetic construct
AQGNGTRPTAQ

SEQ ID NO: 481 - protein, 11 aa, synthetic construct
AQENSVRSSAQ

SEQ ID NO: 482 - protein, 11 aa, synthetic construct
APENHVRSLDQ

SEQ ID NO: 483 - protein, 11 aa, synthetic construct
AQGNGTRPTAQ

SEQ ID NO: 484 - protein, 11 aa, synthetic construct
AQENHTRSSAQ

SEQ ID NO: 485 - protein, 11 aa, synthetic construct
EQENHVRSVAY

SEQ ID NO: 486 - protein, 11 aa, synthetic construct
AQENHVRPNGQ

SEQ ID NO: 487 - protein, 11 aa, synthetic construct
AQGNGTRITAQ

SEQ ID NO: 488 - protein, 11 aa, synthetic construct
DVENHVRNSAQ

SEQ ID NO: 489 - protein, 11 aa, synthetic construct
AQGNGIRPTAQ

SEQ ID NO: 490 - protein, 11 aa, synthetic construct
AQETHVRSSAQ

SEQ ID NO: 491 - protein, 11 aa, synthetic construct
AQENHTRSSAQ

SEQ ID NO: 492 - protein, 11 aa, synthetic construct
AQENHTRSSAQ

SEQ ID NO: 493 - protein, 11 aa, synthetic construct
AQENHTRSSAQ

SEQ ID NO: 494 - protein, 11 aa, synthetic construct
AQENHVRSQND

SEQ ID NO: 495 - protein, 11 aa, synthetic construct
AQLNTTKPIAQ

SEQ ID NO: 496 - protein, 11 aa, synthetic construct
AQLNTTKPIAQ

SEQ ID NO: 497 - protein, 11 aa, synthetic construct
AQENMVRSSAQ

SEQ ID NO: 498 - protein, 11 aa, synthetic construct
AQENMVRSSAQ

SEQ ID NO: 499 - protein, 11 aa, synthetic construct
AQGNGTRPNAQ

SEQ ID NO: 500 - protein, 11 aa, synthetic construct
AQENRVRSSAQ

SEQ ID NO: 501 - protein, 11 aa, synthetic construct
ADENHVRSQAF

SEQ ID NO: 502 - protein, 11 aa, synthetic construct
EQENHVRQSAF

SEQ ID NO: 503 - protein, 11 aa, synthetic construct
AQGNGTRPMAQ

SEQ ID NO: 504 - protein, 11 aa, synthetic construct
AQENHVRPSFG

SEQ ID NO: 505 - protein, 11 aa, synthetic construct
AQENHVRSTEQ

SEQ ID NO: 506 - protein, 11 aa, synthetic construct
AQTNTTKPIAQ

SEQ ID NO: 507 - protein, 11 aa, synthetic construct
AQLNNTKPIAQ

SEQ ID NO: 508 - protein, 11 aa, synthetic construct
AQGNGTRNTAQ

SEQ ID NO: 509 - protein, 11 aa, synthetic construct
AQGNGTRPTAQ

SEQ ID NO: 510 - protein, 11 aa, synthetic construct
AQGNGTRPTAQ

SEQ ID NO: 511 - protein, 11 aa, synthetic construct
AQGNGTRPTAQ

SEQ ID NO: 512 - protein, 11 aa, synthetic construct
AQGNGTRPTAQ

SEQ ID NO: 513 - protein, 11 aa, synthetic construct
AQGNGTKPTAQ

SEQ ID NO: 514 - protein, 11 aa, synthetic construct
AQGNGIRPTAQ

SEQ ID NO: 515 - protein, 11 aa, synthetic construct
KQENQVRSSRQ

SEQ ID NO: 516 - protein, 11 aa, synthetic construct
IQENHVRRSAN

SEQ ID NO: 517 - protein, 11 aa, synthetic construct
AQPNTTKPIAQ

SEQ ID NO: 518 - protein, 11 aa, synthetic construct
HQENHVRSQAE

SEQ ID NO: 519 - protein, 11 aa, synthetic construct
ANENVVRSSAS

SEQ ID NO: 520 - protein, 11 aa, synthetic construct
AQENGVRLSDQ

SEQ ID NO: 521 - protein, 11 aa, synthetic construct
AQGNGTRQTAQ

SEQ ID NO: 522 - protein, 11 aa, synthetic construct
AQGNGTRPNAQ

SEQ ID NO: 523 - protein, 11 aa, synthetic construct
AQGNGTRLTAQ

SEQ ID NO: 524 - protein, 11 aa, synthetic construct
AQENKVRSSAQ

SEQ ID NO: 525 - protein, 11 aa, synthetic construct
AQLNTTKIIAQ

SEQ ID NO: 526 - protein, 11 aa, synthetic construct
AQLNTIKPIAQ

SEQ ID NO: 527 - protein, 11 aa, synthetic construct
AQLNTTKPIAQ

SEQ ID NO: 528 - protein, 11 aa, synthetic construct
AQMNHVRSSAQ

SEQ ID NO: 529 - protein, 11 aa, synthetic construct
AQTPKSPATAQ

SEQ ID NO: 530 - protein, 11 aa, synthetic construct
AQTNHVRSSIK

SEQ ID NO: 531 - protein, 11 aa, synthetic construct
AQENTVRPSFQ

SEQ ID NO: 532 - protein, 11 aa, synthetic construct
AQENHVRSMAQ

SEQ ID NO: 533 - protein, 11 aa, synthetic construct
AQLNTTKPQAQ

SEQ ID NO: 534 - protein, 11 aa, synthetic construct
AQENHVRSDNN

SEQ ID NO: 535 - protein, 11 aa, synthetic construct
AYENHVRSRSQ

SEQ ID NO: 536 - protein, 11 aa, synthetic construct
AQENHVRSCFF

SEQ ID NO: 537 - protein, 11 aa, synthetic construct
AQGNGTRPLAQ

SEQ ID NO: 538 - protein, 11 aa, synthetic construct
AAENHVRSFEQ

SEQ ID NO: 539 - protein, 11 aa, synthetic construct
AQGNGTRPTAQ

SEQ ID NO: 540 - protein, 11 aa, synthetic construct
AQGNGTRMTAQ

SEQ ID NO: 541 - protein, 11 aa, synthetic construct
AAGNHVRSSDQ

SEQ ID NO: 542 - protein, 11 aa, synthetic construct
AQENKVRSSAQ

SEQ ID NO: 543 - protein, 11 aa, synthetic construct
AQLNTIKPIAQ

SEQ ID NO: 544 - protein, 11 aa, synthetic construct
AQENHIRSSAQ

SEQ ID NO: 545 - protein, 11 aa, synthetic construct
AQENHIRSSAQ

SEQ ID NO: 546 - protein, 11 aa, synthetic construct
AGENRVRGSAQ

SEQ ID NO: 547 - protein, 11 aa, synthetic construct
ELQNHVRSSAQ

SEQ ID NO: 548 - protein, 11 aa, synthetic construct
AQENIVRSSAQ

SEQ ID NO: 549 - protein, 11 aa, synthetic construct
AMENHVRSQDQ

SEQ ID NO: 550 - protein, 11 aa, synthetic construct
AQGNGTRPMAQ

SEQ ID NO: 551 - protein, 11 aa, synthetic construct
AQGNGTRPTAQ

SEQ ID NO: 552 - protein, 11 aa, synthetic construct
AQENHVRSMAQ

SEQ ID NO: 553 - protein, 11 aa, synthetic construct
WQENVVRQSAQ

SEQ ID NO: 554 - protein, 11 aa, synthetic construct
AQTNHVRSSAQ

SEQ ID NO: 555 - protein, 11 aa, synthetic construct
RQENHVRSEPQ

SEQ ID NO: 556 - protein, 11 aa, synthetic construct
AKTNHVRSPAQ

SEQ ID NO: 557 - protein, 11 aa, synthetic construct
VQENHVRQTAQ

SEQ ID NO: 558 - protein, 11 aa, synthetic construct
ADENHVRSNAA

SEQ ID NO: 559 - protein, 11 aa, synthetic construct
AQLNTTKPRAQ

SEQ ID NO: 560 - protein, 11 aa, synthetic construct
AQENHVRSENT

SEQ ID NO: 561 - protein, 11 aa, synthetic construct
AQENHTRSSAQ

SEQ ID NO: 562 - protein, 11 aa, synthetic construct
AQLNTTRPIAQ

SEQ ID NO: 563 - protein, 11 aa, synthetic construct
AQENFVRSRIQ

SEQ ID NO: 564 - protein, 11 aa, synthetic construct
MEGNHVRSSAQ

SEQ ID NO: 565 - protein, 11 aa, synthetic construct
AQGNGTRITAQ

SEQ ID NO: 566 - protein, 11 aa, synthetic construct
AQGNGIRPTAQ

SEQ ID NO: 567 - protein, 11 aa, synthetic construct
MQENHVRQSAD

SEQ ID NO: 568 - protein, 11 aa, synthetic construct
AQENHVRPTAN

SEQ ID NO: 569 - protein, 11 aa, synthetic construct
AQNNHVRESAM

SEQ ID NO: 570 - protein, 11 aa, synthetic construct
YQENHVRSNNQ

SEQ ID NO: 571 - protein, 11 aa, synthetic construct
AQENTVRSSAQ

SEQ ID NO: 572 - protein, 11 aa, synthetic construct
TQENHVRGSQQ

SEQ ID NO: 573 - protein, 11 aa, synthetic construct
MQENHVRQTAQ

SEQ ID NO: 574 - protein, 11 aa, synthetic construct
AQENHVRSNAQ

SEQ ID NO: 575 - protein, 11 aa, synthetic construct
AQENHVRSNAQ

SEQ ID NO: 576 - protein, 11 aa, synthetic construct
TQENHVRPSGQ

SEQ ID NO: 577 - protein, 11 aa, synthetic construct
AQENHVRSFDQ

SEQ ID NO: 578 - protein, 11 aa, synthetic construct
AQENQVRSSAQ

SEQ ID NO: 579 - protein, 11 aa, synthetic construct
AQENHIRSSAQ

SEQ ID NO: 580 - protein, 11 aa, synthetic construct
AQENSVRSSAQ

SEQ ID NO: 581 - protein, 11 aa, synthetic construct
GQENNVRNSAQ

SEQ ID NO: 582 - protein, 11 aa, synthetic construct
EQENHVRSQNQ

SEQ ID NO: 583 - protein, 11 aa, synthetic construct
ADENRVRSSSQ

SEQ ID NO: 584 - protein, 11 aa, synthetic construct
DQENHVRSNAE

SEQ ID NO: 585 - protein, 11 aa, synthetic construct
AQGNGTRNTAQ

SEQ ID NO: 586 - protein, 11 aa, synthetic construct
ESENHVRSFAQ

SEQ ID NO: 587 - protein, 11 aa, synthetic construct
YWENHVRSFAQ

SEQ ID NO: 588 - protein, 11 aa, synthetic construct
VQQNHVRSSEQ

SEQ ID NO: 589 - protein, 11 aa, synthetic construct
ADGNHVRSLAQ

SEQ ID NO: 590 - protein, 11 aa, synthetic construct
ANENHVRVQAQ

SEQ ID NO: 591 - protein, 11 aa, synthetic construct
DQENRVRVSAQ

SEQ ID NO: 592 - protein, 11 aa, synthetic construct
ADENHVRSLTQ

SEQ ID NO: 593 - protein, 11 aa, synthetic construct
AQGNGTRPNAQ

SEQ ID NO: 594 - protein, 11 aa, synthetic construct
AQENHVRSTAD

SEQ ID NO: 595 - protein, 11 aa, synthetic construct
ALENHVRQQAQ

SEQ ID NO: 596 - protein, 11 aa, synthetic construct
AQGNGTRPTAQ

SEQ ID NO: 597 - protein, 11 aa, synthetic construct
AAENHVRSLAQ

SEQ ID NO: 598 - protein, 11 aa, synthetic construct
PLENHVRSEAQ

SEQ ID NO: 599 - protein, 11 aa, synthetic construct
AQENRVRSQAR

SEQ ID NO: 600 - protein, 11 aa, synthetic construct
AQENHTRSSAQ

SEQ ID NO: 601 - protein, 11 aa, synthetic construct
AQENTVRSLAW

SEQ ID NO: 602 - protein, 11 aa, synthetic construct
MDENHVRSNAQ

SEQ ID NO: 603 - protein, 11 aa, synthetic construct
AQGNGTKPTAQ

SEQ ID NO: 604 - protein, 11 aa, synthetic construct
HQENHVRSQAD

SEQ ID NO: 605 - protein, 11 aa, synthetic construct
DQGNVVRSSAQ

SEQ ID NO: 606 - protein, 11 aa, synthetic construct
DYENHVRSTAQ

SEQ ID NO: 607 - protein, 11 aa, synthetic construct
VLENHVRSQAQ

SEQ ID NO: 608 - protein, 11 aa, synthetic construct
AQTSKSPATAQ

SEQ ID NO: 609 - protein, 11 aa, synthetic construct
AQGNNTRPTAQ

SEQ ID NO: 610 - protein, 11 aa, synthetic construct
AQGNGTRPTAQ

SEQ ID NO: 611 - protein, 11 aa, synthetic construct
ALENHVRSTNQ

SEQ ID NO: 612 - protein, 11 aa, synthetic construct
AQENHVRSLAP

SEQ ID NO: 613 - protein, 11 aa, synthetic construct
MQENHVRSVAS

SEQ ID NO: 614 - protein, 11 aa, synthetic construct
AQENHIRSSAQ

SEQ ID NO: 615 - protein, 11 aa, synthetic construct
AQENHVRSLAQ

SEQ ID NO: 616 - protein, 11 aa, synthetic construct
AQENRVRSSAQ

SEQ ID NO: 617 - protein, 11 aa, synthetic construct
ANENHVRFSRQ

SEQ ID NO: 618 - protein, 11 aa, synthetic construct
AQGNGTRNTAQ

SEQ ID NO: 619 - protein, 11 aa, synthetic construct
AQGNGTRMTAQ

SEQ ID NO: 620 - protein, 11 aa, synthetic construct
DQENVVRPSAQ

SEQ ID NO: 621 - protein, 11 aa, synthetic construct
HQENHVRNSAR

SEQ ID NO: 622 - protein, 11 aa, synthetic construct
AQLNHVRSSAQ

SEQ ID NO: 623 - protein, 11 aa, synthetic construct
DGENHVRSFAQ

SEQ ID NO: 624 - protein, 11 aa, synthetic construct
AQENHVRQSDQ

SEQ ID NO: 625 - protein, 11 aa, synthetic construct
ALENHVRSVAQ

SEQ ID NO: 626 - protein, 11 aa, synthetic construct
WQENHVRSLQQ

SEQ ID NO: 627 - protein, 11 aa, synthetic construct
AQENHVRTSAK

SEQ ID NO: 628 - protein, 11 aa, synthetic construct
ATENHVRQLAQ

SEQ ID NO: 629 - protein, 11 aa, synthetic construct
AQENHVRSMAQ

SEQ ID NO: 630 - protein, 11 aa, synthetic construct
AVENHVRQQAQ

SEQ ID NO: 631 - protein, 11 aa, synthetic construct
AQLLRDKSSAQ

SEQ ID NO: 632 - protein, 11 aa, synthetic construct
TNENHVRSEAQ

SEQ ID NO: 633 - protein, 11 aa, synthetic construct
AQGNGTRQTAQ

SEQ ID NO: 634 - protein, 11 aa, synthetic construct
AQGNGTRNTAQ

SEQ ID NO: 635 - protein, 11 aa, synthetic construct
AMQNHVRSSWQ

SEQ ID NO: 636 - protein, 11 aa, synthetic construct
AQGNGTRITAQ

SEQ ID NO: 637 - protein, 11 aa, synthetic construct
MTENHVRGSAQ

SEQ ID NO: 638 - protein, 11 aa, synthetic construct
AQGNGTRPTAQ

SEQ ID NO: 639 - protein, 11 aa, synthetic construct
MQENHVRNEAQ

SEQ ID NO: 640 - protein, 11 aa, synthetic construct
AQENNVRSSAQ

SEQ ID NO: 641 - protein, 11 aa, synthetic construct
AQENNVRSSAQ

SEQ ID NO: 642 - protein, 11 aa, synthetic construct
WQENQVRPSAQ

SEQ ID NO: 643 - protein, 11 aa, synthetic construct
AHENHVRSFAR

SEQ ID NO: 644 - protein, 11 aa, synthetic construct
AQENMVRSSAQ

SEQ ID NO: 645 - protein, 11 aa, synthetic construct
AQENGVRSLPQ

SEQ ID NO: 646 - protein, 11 aa, synthetic construct
AQENHVRSTMD

SEQ ID NO: 647 - protein, 11 aa, synthetic construct
CQENHVRGSAG

SEQ ID NO: 648 - protein, 11 aa, synthetic construct
AQTNHVRPSDQ

SEQ ID NO: 649 - protein, 11 aa, synthetic construct
DYENHVRSVAQ

SEQ ID NO: 650 - protein, 11 aa, synthetic construct
DQENHVRSPAQ

SEQ ID NO: 651 - protein, 11 aa, synthetic construct
PQENHVRPSAD

SEQ ID NO: 652 - protein, 11 aa, synthetic construct
AQENMVRSSAQ

SEQ ID NO: 653 - protein, 11 aa, synthetic construct
ARENHVRSTDQ

SEQ ID NO: 654 - protein, 11 aa, synthetic construct
AQENHVRNSDL

SEQ ID NO: 655 - protein, 11 aa, synthetic construct
AQENMVRSSAQ

SEQ ID NO: 656 - protein, 11 aa, synthetic construct
AHENHVRSQAD

SEQ ID NO: 657 - protein, 11 aa, synthetic construct
AQENHVRGFDQ

SEQ ID NO: 658 - protein, 11 aa, synthetic construct
QAENHVRSLAQ

SEQ ID NO: 659 - protein, 11 aa, synthetic construct
AQENHVRSLAQ

SEQ ID NO: 660 - protein, 11 aa, synthetic construct
AQGNGTRPTAQ

SEQ ID NO: 661 - protein, 11 aa, synthetic construct
ALENHVRSTAD

SEQ ID NO: 662 - protein, 11 aa, synthetic construct
IQENHVRPQAQ

SEQ ID NO: 663 - protein, 11 aa, synthetic construct
AQGNGTKPTAQ

SEQ ID NO: 664 - protein, 11 aa, synthetic construct
AQENGVRSSDG

SEQ ID NO: 665 - protein, 11 aa, synthetic construct
ANENHVRPTAQ

SEQ ID NO: 666 - protein, 11 aa, synthetic construct
AQENHVRSLAQ

SEQ ID NO: 667 - protein, 11 aa, synthetic construct
AHENHVRSLEQ

SEQ ID NO: 668 - protein, 11 aa, synthetic construct
DQENHVRSEAQ

SEQ ID NO: 669 - protein, 11 aa, synthetic construct
AEENHVRSVAQ

SEQ ID NO: 670 - protein, 11 aa, synthetic construct
DTENHVRSTAQ

SEQ ID NO: 671 - protein, 11 aa, synthetic construct
AEENRVRSSAC

SEQ ID NO: 672 - protein, 11 aa, synthetic construct
LQENHVRSGAQ

SEQ ID NO: 673 - protein, 11 aa, synthetic construct
AQGNGTRPQAQ

SEQ ID NO: 674 - protein, 11 aa, synthetic construct
ANENHVRPSAQ

SEQ ID NO: 675 - protein, 11 aa, synthetic construct
AQENHTRSSAQ

SEQ ID NO: 676 - protein, 11 aa, synthetic construct
AFENHVRSTAI

SEQ ID NO: 677 - protein, 11 aa, synthetic construct
TQENHVRTQAQ

SEQ ID NO: 678 - protein, 11 aa, synthetic construct
EQGNHVRSSAF

SEQ ID NO: 679 - protein, 11 aa, synthetic construct
AQENHNRSSAQ

SEQ ID NO: 680 - protein, 11 aa, synthetic construct
DQENHVRFLAQ

SEQ ID NO: 681 - protein, 11 aa, synthetic construct
TMENHVRTSAQ

SEQ ID NO: 682 - protein, 11 aa, synthetic construct
NQENHVRSNAQ

SEQ ID NO: 683 - protein, 11 aa, synthetic construct
AQENHVRSTAQ

SEQ ID NO: 684 - protein, 11 aa, synthetic construct
AQLNMTKPIAQ

SEQ ID NO: 685 - protein, 11 aa, synthetic construct
HQRNHVRSSDQ

SEQ ID NO: 686 - protein, 11 aa, synthetic construct
HMENHVRSEAQ

SEQ ID NO: 687 - protein, 11 aa, synthetic construct
DQENNVRPSAQ

SEQ ID NO: 688 - protein, 11 aa, synthetic construct
AYGNHVRSTAQ

SEQ ID NO: 689 - protein, 11 aa, synthetic construct
AQLNTTKPIAQ

SEQ ID NO: 690 - protein, 11 aa, synthetic construct
ALENHVRSNDQ

SEQ ID NO: 691 - protein, 11 aa, synthetic construct
PMENHVRPSAQ

SEQ ID NO: 692 - protein, 11 aa, synthetic construct
NQENHVRQSAQ

SEQ ID NO: 693 - protein, 11 aa, synthetic construct
AQLNTTKPIAQ

SEQ ID NO: 694 - protein, 11 aa, synthetic construct
AQGNGTRTTAQ

SEQ ID NO: 695 - protein, 11 aa, synthetic construct
AAENHVRSVAN

SEQ ID NO: 696 - protein, 11 aa, synthetic construct
AEENHVRPSGQ

SEQ ID NO: 697 - protein, 11 aa, synthetic construct
ADENHVRSQAA

SEQ ID NO: 698 - protein, 11 aa, synthetic construct
AQENSVRSSAQ

SEQ ID NO: 699 - protein, 11 aa, synthetic construct
AAENHVRSVAQ

SEQ ID NO: 700 - protein, 11 aa, synthetic construct
FYVNHVRSSAQ

SEQ ID NO: 701 - protein, 11 aa, synthetic construct
TQENNVRSSAM

SEQ ID NO: 702 - protein, 11 aa, synthetic construct
AQENHVRSTAP

SEQ ID NO: 703 - protein, 11 aa, synthetic construct
AAENHVRSNAQ

SEQ ID NO: 704 - protein, 11 aa, synthetic construct
HEENHVRNSAQ

SEQ ID NO: 705 - protein, 11 aa, synthetic construct
MQENTVRSSAI

SEQ ID NO: 706 - protein, 11 aa, synthetic construct
VFENHVRNSAQ

SEQ ID NO: 707 - protein, 11 aa, synthetic construct
AQGNGTRPTAQ

SEQ ID NO: 708 - protein, 11 aa, synthetic construct
AAENHVRPTAQ

SEQ ID NO: 709 - protein, 11 aa, synthetic construct
AQENLVRSSAQ

SEQ ID NO: 710 - protein, 11 aa, synthetic construct
AMENHVRQNAQ

SEQ ID NO: 711 - protein, 11 aa, synthetic construct
GDENHVRNSAQ

SEQ ID NO: 712 - protein, 11 aa, synthetic construct
AAENHVRQSNQ

SEQ ID NO: 713 - protein, 11 aa, synthetic construct
DPENRVRSSAQ

SEQ ID NO: 714 - protein, 11 aa, synthetic construct
GQENHVRSQAN

SEQ ID NO: 715 - protein, 11 aa, synthetic construct
AQENHVRNSAQ

SEQ ID NO: 716 - protein, 11 aa, synthetic construct
AEENTVRSSAQ

SEQ ID NO: 717 - protein, 11 aa, synthetic construct
AQGNGIRPTAQ

SEQ ID NO: 718 - protein, 11 aa, synthetic construct
PTENHVRNSAQ

SEQ ID NO: 719 - protein, 11 aa, synthetic construct
PQENHVRTSAD

SEQ ID NO: 720 - protein, 11 aa, synthetic construct
AQLLRDKSTAQ

SEQ ID NO: 721 - protein, 11 aa, synthetic construct
EQENHVRNPAQ

SEQ ID NO: 722 - protein, 11 aa, synthetic construct
AQENHVRQQAG

SEQ ID NO: 723 - protein, 11 aa, synthetic construct
AVENTVRSQAQ

SEQ ID NO: 724 - protein, 11 aa, synthetic construct
AQGNHVRSNDQ

SEQ ID NO: 725 - protein, 11 aa, synthetic construct
AQENTVRPSHQ

SEQ ID NO: 726 - protein, 11 aa, synthetic construct
AQENRVRSSSC

SEQ ID NO: 727 - protein, 11 aa, synthetic construct
PQENHVRSPSQ

SEQ ID NO: 728 - protein, 11 aa, synthetic construct
APENHVRSLSQ

SEQ ID NO: 729 - protein, 11 aa, synthetic construct
EQPNHVRSQAQ

SEQ ID NO: 730 - protein, 11 aa, synthetic construct
AQGNGTRNTAQ

SEQ ID NO: 731 - protein, 11 aa, synthetic construct
AQENKVRSSAQ

SEQ ID NO: 732 - protein, 11 aa, synthetic construct
VQENHVRSTAQ

SEQ ID NO: 733 - protein, 11 aa, synthetic construct
PQENQVRSSAD

SEQ ID NO: 734 - protein, 11 aa, synthetic construct
IVENHVRSVAQ

SEQ ID NO: 735 - protein, 11 aa, synthetic construct
AYENFVRSLAQ

SEQ ID NO: 736 - protein, 11 aa, synthetic construct
VQENHVRSNAD

SEQ ID NO: 737 - protein, 11 aa, synthetic construct
AQGNGSRPTAQ

SEQ ID NO: 738 - protein, 11 aa, synthetic construct
AQGNRVRSSEQ

SEQ ID NO: 739 - protein, 11 aa, synthetic construct
AQENHIRSSAQ

SEQ ID NO: 740 - protein, 11 aa, synthetic construct
AQGNGTRLTAQ

SEQ ID NO: 741 - protein, 11 aa, synthetic construct
TQENHVRSEAQ

SEQ ID NO: 742 - protein, 11 aa, synthetic construct
QQENQVRSNAQ

SEQ ID NO: 743 - protein, 11 aa, synthetic construct
IQENVVRPSAQ

SEQ ID NO: 744 - protein, 11 aa, synthetic construct
FQENHVRSRSQ

SEQ ID NO: 745 - protein, 11 aa, synthetic construct
AVTNHVRRSAQ

SEQ ID NO: 746 - protein, 11 aa, synthetic construct
DQENNVRSSAG

SEQ ID NO: 747 - protein, 11 aa, synthetic construct
QEENRVRSSAQ

SEQ ID NO: 748 - protein, 11 aa, synthetic construct
DQENRVRSSAD

SEQ ID NO: 749 - protein, 11 aa, synthetic construct
SNENHVRSVAQ

SEQ ID NO: 750 - protein, 11 aa, synthetic construct
PQENTVRSSAQ

SEQ ID NO: 751 - protein, 11 aa, synthetic construct
TQENHVRQSAL

SEQ ID NO: 752 - protein, 11 aa, synthetic construct
QQENHVRSEAF

SEQ ID NO: 753 - protein, 11 aa, synthetic construct
MNENHVRQSAQ

SEQ ID NO: 754 - protein, 11 aa, synthetic construct
ASENHVRQSAN

SEQ ID NO: 755 - protein, 11 aa, synthetic construct
DQENQVRSLAQ

SEQ ID NO: 756 - protein, 11 aa, synthetic construct
ASENTVRSVAQ

SEQ ID NO: 757 - protein, 11 aa, synthetic construct
EYENHVRSNAQ

SEQ ID NO: 758 - protein, 11 aa, synthetic construct
AAENHVRSTTQ

SEQ ID NO: 759 - protein, 11 aa, synthetic construct
QQENHVRSVAQ

SEQ ID NO: 760 - protein, 11 aa, synthetic construct
AQENHVRSTNT

SEQ ID NO: 761 - protein, 11 aa, synthetic construct
AEENHVRSVAT

SEQ ID NO: 762 - protein, 11 aa, synthetic construct
PNENTVRSSAQ

SEQ ID NO: 763 - protein, 11 aa, synthetic construct
AQENHVRSTLS

SEQ ID NO: 764 - protein, 11 aa, synthetic construct
AQLNTIKPIAQ

SEQ ID NO: 765 - protein, 11 aa, synthetic construct
AQENHVRSNLE

SEQ ID NO: 766 - protein, 11 aa, synthetic construct
AQENKVRSSAQ

SEQ ID NO: 767 - protein, 11 aa, synthetic construct
AQENNVRSSAQ

SEQ ID NO: 768 - protein, 11 aa, synthetic construct
AQENRVRSSAQ

SEQ ID NO: 769 - protein, 11 aa, synthetic construct
VQENHVRNSAN

SEQ ID NO: 770 - protein, 11 aa, synthetic construct
MQENQVRSSPQ

SEQ ID NO: 771 - protein, 11 aa, synthetic construct
TNENHVRVSAQ

SEQ ID NO: 772 - protein, 11 aa, synthetic construct
AQENNVRSSAQ

SEQ ID NO: 773 - protein, 11 aa, synthetic construct
AQENHVRSQFD

SEQ ID NO: 774 - protein, 11 aa, synthetic construct
AQENHVRPSNN

SEQ ID NO: 775 - protein, 11 aa, synthetic construct
AQENHVRTVAD

SEQ ID NO: 776 - protein, 11 aa, synthetic construct
DAENHVRSPAQ

SEQ ID NO: 777 - protein, 11 aa, synthetic construct
IQENHVRSQAG

SEQ ID NO: 778 - protein, 11 aa, synthetic construct
AEENRVRSSNQ

SEQ ID NO: 779 - protein, 11 aa, synthetic construct
QQENHVRNPAQ

SEQ ID NO: 780 - protein, 11 aa, synthetic construct
QQVNHVRSFAQ

SEQ ID NO: 781 - protein, 11 aa, synthetic construct
VQENHVRNSAE

SEQ ID NO: 782 - protein, 11 aa, synthetic construct
ANENHVRSLAQ

SEQ ID NO: 783 - protein, 11 aa, synthetic construct
AQENHVRQSHD

SEQ ID NO: 784 - protein, 11 aa, synthetic construct
AVENHVRNSDQ

SEQ ID NO: 785 - protein, 11 aa, synthetic construct
AQTNTTKPIAQ

SEQ ID NO: 786 - protein, 11 aa, synthetic construct
AAENHVRSQAE

SEQ ID NO: 787 - protein, 11 aa, synthetic construct
AQLNTTKLIAQ

SEQ ID NO: 788 - protein, 11 aa, synthetic construct
QQENTVRSSAH

SEQ ID NO: 789 - protein, 11 aa, synthetic construct
HQENTVRSSAQ

SEQ ID NO: 790 - protein, 11 aa, synthetic construct
APENHVRSTAQ

SEQ ID NO: 791 - protein, 11 aa, synthetic construct
AIENHVRSTVQ

SEQ ID NO: 792 - protein, 11 aa, synthetic construct
AQENGVRSQIQ

SEQ ID NO: 793 - protein, 11 aa, synthetic construct
MQENHVRNSNQ

SEQ ID NO: 794 - protein, 11 aa, synthetic construct
IQENHVRSNAG

SEQ ID NO: 795 - protein, 11 aa, synthetic construct
AQGNGTRPQAQ

SEQ ID NO: 796 - protein, 11 aa, synthetic construct
AQGNGTKPTAQ

SEQ ID NO: 797 - protein, 11 aa, synthetic construct
AVENHVRSQNQ

SEQ ID NO: 798 - protein, 7 aa, synthetic construct
NGNGQNQ

SEQ ID NO: 799 - protein, 7 aa, synthetic construct
NVSGQNQ

SEQ ID NO: 800 - protein, 7 aa, synthetic construct
PGSGQNQ

SEQ ID NO: 801 - protein, 7 aa, synthetic construct
NGSGQNQ

SEQ ID NO: 802 - protein, 7 aa, synthetic construct
NGSGQNQ

SEQ ID NO: 803 - protein, 7 aa, synthetic construct
PGSGQNQ

SEQ ID NO: 804 - protein, 7 aa, synthetic construct
HGSGQNQ

SEQ ID NO: 805 - protein, 7 aa, synthetic construct
GGSGQNQ

SEQ ID NO: 806 - protein, 7 aa, synthetic construct
GGSGQNQ

SEQ ID NO: 807 - protein, 7 aa, synthetic construct
NGFGQNQ

SEQ ID NO: 808 - protein, 7 aa, synthetic construct
LGSGQNQ

SEQ ID NO: 809 - protein, 7 aa, synthetic construct
NGSGQIQ

SEQ ID NO: 810 - protein, 7 aa, synthetic construct
NSSGQNQ

SEQ ID NO: 811 - protein, 7 aa, synthetic construct
NGSGQNQ

SEQ ID NO: 812 - protein, 7 aa, synthetic construct
LGSGQNQ

SEQ ID NO: 813 - protein, 7 aa, synthetic construct
SGSGQNQ

SEQ ID NO: 814 - protein, 7 aa, synthetic construct
SGSGQNQ

SEQ ID NO: 815 - protein, 7 aa, synthetic construct
NGSGQNV

SEQ ID NO: 816 - protein, 7 aa, synthetic construct
NGSLQNQ

SEQ ID NO: 817 - protein, 7 aa, synthetic construct
NGSGQNQ

SEQ ID NO: 818 - protein, 7 aa, synthetic construct
NGSGQNV

SEQ ID NO: 819 - protein, 7 aa, synthetic construct
NGSTQNQ

SEQ ID NO: 820 - protein, 7 aa, synthetic construct
NGSTQNQ

SEQ ID NO: 821 - protein, 7 aa, synthetic construct
NGSGQNQ

SEQ ID NO: 822 - protein, 7 aa, synthetic construct
VGSGQNQ

SEQ ID NO: 823 - protein, 7 aa, synthetic construct
NGSAQNQ

SEQ ID NO: 824 - protein, 7 aa, synthetic construct
NGSGQNQ

SEQ ID NO: 825 - protein, 7 aa, synthetic construct
NGSGQMQ

SEQ ID NO: 826 - protein, 7 aa, synthetic construct
SGSGQNQ

SEQ ID NO: 827 - protein, 7 aa, synthetic construct
YGSGQNQ

SEQ ID NO: 828 - protein, 7 aa, synthetic construct
NGSGFNQ

SEQ ID NO: 829 - protein, 7 aa, synthetic construct
NLSGQNQ

SEQ ID NO: 830 - protein, 7 aa, synthetic construct
NGSLQNQ

SEQ ID NO: 831 - protein, 7 aa, synthetic construct
GGSGQNQ

SEQ ID NO: 832 - protein, 7 aa, synthetic construct
DGSGQNQ

SEQ ID NO: 833 - protein, 7 aa, synthetic construct
IGSGQNQ

SEQ ID NO: 834 - protein, 7 aa, synthetic construct
NGSGQNQ

SEQ ID NO: 835 - protein, 7 aa, synthetic construct
NGIGQNQ

SEQ ID NO: 836 - protein, 7 aa, synthetic construct
NGSNQNQ

SEQ ID NO: 837 - protein, 7 aa, synthetic construct
AGSGQNQ

SEQ ID NO: 838 - protein, 7 aa, synthetic construct
NGSGQNQ

SEQ ID NO: 839 - protein, 7 aa, synthetic construct
NGSGQNQ

SEQ ID NO: 840 - protein, 7 aa, synthetic construct
MGSGQNQ

SEQ ID NO: 841 - protein, 7 aa, synthetic construct
WGSGQNQ

SEQ ID NO: 842 - protein, 7 aa, synthetic construct
NGSSQNQ

SEQ ID NO: 843 - protein, 7 aa, synthetic construct
EGSGQNQ

SEQ ID NO: 844 - protein, 7 aa, synthetic construct
TGSGQNQ

SEQ ID NO: 845 - protein, 7 aa, synthetic construct
NGSVQNQ

SEQ ID NO: 846 - protein, 7 aa, synthetic construct
VGSGQNQ

SEQ ID NO: 847 - protein, 7 aa, synthetic construct
NGSVQNQ

SEQ ID NO: 848 - protein, 7 aa, synthetic construct
NYSGQNQ

SEQ ID NO: 849 - protein, 7 aa, synthetic construct
NGSGQNQ

SEQ ID NO: 850 - protein, 7 aa, synthetic construct
NGSGQNQ

SEQ ID NO: 851 - protein, 7 aa, synthetic construct
NGSIQNQ

SEQ ID NO: 852 - protein, 7 aa, synthetic construct
NGSGQNL

SEQ ID NO: 853 - protein, 7 aa, synthetic construct
NGSGQNV

SEQ ID NO: 854 - protein, 7 aa, synthetic construct
NGSGQNV

SEQ ID NO: 855 - protein, 7 aa, synthetic construct
NGSHQNQ

SEQ ID NO: 856 - protein, 7 aa, synthetic construct
NGSGQNQ

SEQ ID NO: 857 - protein, 7 aa, synthetic construct
NGSGLNQ

SEQ ID NO: 858 - protein, 7 aa, synthetic construct
NLSGQNQ

SEQ ID NO: 859 - protein, 7 aa, synthetic construct
VGSGQNQ

SEQ ID NO: 860 - protein, 7 aa, synthetic construct
VGSGQNQ

SEQ ID NO: 861 - protein, 7 aa, synthetic construct
NGVGQNQ

SEQ ID NO: 862 - protein, 7 aa, synthetic construct
NGSGQNQ

SEQ ID NO: 863 - protein, 7 aa, synthetic construct
NGSGQNQ

SEQ ID NO: 864 - protein, 7 aa, synthetic construct
IGSGQNQ

SEQ ID NO: 865 - protein, 7 aa, synthetic construct
NGSGLNQ

SEQ ID NO: 866 - protein, 7 aa, synthetic construct
NGSGLNQ

SEQ ID NO: 867 - protein, 7 aa, synthetic construct
NGSGQNQ

SEQ ID NO: 868 - protein, 7 aa, synthetic construct
NGSGQNQ

SEQ ID NO: 869 - protein, 7 aa, synthetic construct
NGSEQNQ

SEQ ID NO: 870 - protein, 7 aa, synthetic construct
NGSSQNQ

SEQ ID NO: 871 - protein, 7 aa, synthetic construct
NGSGQNG

SEQ ID NO: 872 - protein, 7 aa, synthetic construct
NGSGQNQ

SEQ ID NO: 873 - protein, 7 aa, synthetic construct
FGSGQNQ

SEQ ID NO: 874 - protein, 7 aa, synthetic construct
YGSGQNQ

SEQ ID NO: 875 - protein, 7 aa, synthetic construct
WGSGQNQ

SEQ ID NO: 876 - protein, 7 aa, synthetic construct
WGSGQNQ

SEQ ID NO: 877 - protein, 7 aa, synthetic construct
NGSGQNQ

SEQ ID NO: 878 - protein, 7 aa, synthetic construct
NGSGQNQ

SEQ ID NO: 879 - protein, 7 aa, synthetic construct
NGSGQNQ

SEQ ID NO: 880 - protein, 7 aa, synthetic construct
NGSDQNQ

SEQ ID NO: 881 - protein, 7 aa, synthetic construct
NGSPQNQ

SEQ ID NO: 882 - protein, 7 aa, synthetic construct
VGSGQNQ

SEQ ID NO: 883 - protein, 7 aa, synthetic construct
NGSGQSQ

SEQ ID NO: 884 - protein, 7 aa, synthetic construct
NGSAQNQ

SEQ ID NO: 885 - protein, 7 aa, synthetic construct
NGSGQFQ

SEQ ID NO: 886 - protein, 7 aa, synthetic construct
NGSGQNQ

SEQ ID NO: 887 - protein, 7 aa, synthetic construct
NGSGQNQ

SEQ ID NO: 888 - protein, 7 aa, synthetic construct
IGSGQNQ

SEQ ID NO: 889 - protein, 7 aa, synthetic construct
FGSGQNQ

SEQ ID NO: 890 - protein, 7 aa, synthetic construct
NSSGQNQ

SEQ ID NO: 891 - protein, 7 aa, synthetic construct
NGSGQNS

SEQ ID NO: 892 - protein, 7 aa, synthetic construct
NGSGQNA

SEQ ID NO: 893 - protein, 7 aa, synthetic construct
NGSGQNS

SEQ ID NO: 894 - protein, 7 aa, synthetic construct
NGSGQLQ

SEQ ID NO: 895 - protein, 7 aa, synthetic construct
SGSGQNQ

SEQ ID NO: 896 - protein, 7 aa, synthetic construct
NGSGQGQ

SEQ ID NO: 897 - protein, 7 aa, synthetic construct
NGSGQLQ

SEQ ID NO: 898 - protein, 7 aa, synthetic construct
NGSGQLQ

SEQ ID NO: 899 - protein, 7 aa, synthetic construct
NGSGQLQ

SEQ ID NO: 900 - protein, 7 aa, synthetic construct
NGSHQNQ

SEQ ID NO: 901 - protein, 7 aa, synthetic construct
NGTGQNQ

SEQ ID NO: 902 - protein, 7 aa, synthetic construct
QGSGQNQ

SEQ ID NO: 903 - protein, 7 aa, synthetic construct
NGSGQNF

SEQ ID NO: 904 - protein, 7 aa, synthetic construct
NHSGQNQ

SEQ ID NO: 905 - protein, 7 aa, synthetic construct
VGSGQNQ

SEQ ID NO: 906 - protein, 7 aa, synthetic construct
GGSGQNQ

SEQ ID NO: 907 - protein, 7 aa, synthetic construct
NGSNQNQ

SEQ ID NO: 908 - protein, 7 aa, synthetic construct
NGSGQNQ

SEQ ID NO: 909 - protein, 7 aa, synthetic construct
NGSGQNQ

SEQ ID NO: 910 - protein, 7 aa, synthetic construct
EGSGQNQ

SEQ ID NO: 911 - protein, 7 aa, synthetic construct
TGSGQNQ

SEQ ID NO: 912 - protein, 7 aa, synthetic construct
NSSGQNQ

SEQ ID NO: 913 - protein, 7 aa, synthetic construct
NGSGQNQ

SEQ ID NO: 914 - protein, 7 aa, synthetic construct
NGSGQNQ

SEQ ID NO: 915 - protein, 7 aa, synthetic construct
FGSGQNQ

SEQ ID NO: 916 - protein, 7 aa, synthetic construct
NGSGQDQ

SEQ ID NO: 917 - protein, 7 aa, synthetic construct
NGSGQNQ

SEQ ID NO: 918 - protein, 7 aa, synthetic construct
NGSGQNQ

SEQ ID NO: 919 - protein, 7 aa, synthetic construct
NGSGQNQ

SEQ ID NO: 920 - protein, 7 aa, synthetic construct
NGSGQNQ

SEQ ID NO: 921 - protein, 7 aa, synthetic construct
NGSGQNQ

SEQ ID NO: 922 - protein, 7 aa, synthetic construct
NGSGQNQ

SEQ ID NO: 923 - protein, 7 aa, synthetic construct
CGSGQNQ

SEQ ID NO: 924 - protein, 7 aa, synthetic construct
DGSGQNQ

SEQ ID NO: 925 - protein, 7 aa, synthetic construct
DGSGQNQ

SEQ ID NO: 926 - protein, 7 aa, synthetic construct
NGSGQNQ

SEQ ID NO: 927 - protein, 7 aa, synthetic construct
NGSGQNQ

SEQ ID NO: 928 - protein, 7 aa, synthetic construct
NGSGQNQ

SEQ ID NO: 929 - protein, 7 aa, synthetic construct
NGSGQNQ

SEQ ID NO: 930 - protein, 7 aa, synthetic construct
NGSSQNQ

SEQ ID NO: 931 - protein, 7 aa, synthetic construct
NGYGQNQ

SEQ ID NO: 932 - protein, 7 aa, synthetic construct
MGSGQNQ

SEQ ID NO: 933 - protein, 7 aa, synthetic construct
NGSGQNQ

SEQ ID NO: 934 - protein, 7 aa, synthetic construct
NGSGQRQ

SEQ ID NO: 935 - protein, 7 aa, synthetic construct
NGGGQNQ

SEQ ID NO: 936 - protein, 7 aa, synthetic construct
NGGGQNQ

SEQ ID NO: 937 - protein, 7 aa, synthetic construct
NGSGQNQ

SEQ ID NO: 938 - protein, 7 aa, synthetic construct
NGSGQNQ

SEQ ID NO: 939 - protein, 7 aa, synthetic construct
NGSGQLQ

SEQ ID NO: 940 - protein, 7 aa, synthetic construct
NGSGQTQ

SEQ ID NO: 941 - protein, 7 aa, synthetic construct
PGSGQNQ

SEQ ID NO: 942 - protein, 7 aa, synthetic construct
NGSPQNQ

SEQ ID NO: 943 - protein, 7 aa, synthetic construct
NGSNQNQ

SEQ ID NO: 944 - protein, 7 aa, synthetic construct
NGSGQNQ

SEQ ID NO: 945 - protein, 7 aa, synthetic construct
NGSGQNQ

SEQ ID NO: 946 - protein, 7 aa, synthetic construct
GGSGQNQ

SEQ ID NO: 947 - protein, 7 aa, synthetic construct
NGSGQNQ

SEQ ID NO: 948 - protein, 7 aa, synthetic construct
MGSGQNQ

SEQ ID NO: 949 - protein, 7 aa, synthetic construct
NGSGQNQ

SEQ ID NO: 950 - protein, 7 aa, synthetic construct
NGSAQNQ

SEQ ID NO: 951 - protein, 7 aa, synthetic construct
NGSGQNL

SEQ ID NO: 952 - protein, 7 aa, synthetic construct
NGSGQNQ

SEQ ID NO: 953 - protein, 7 aa, synthetic construct
NGSGQNQ

SEQ ID NO: 954 - protein, 7 aa, synthetic construct
NGSGQNV

SEQ ID NO: 955 - protein, 7 aa, synthetic construct
NGSAQNQ

SEQ ID NO: 956 - protein, 7 aa, synthetic construct
TGSGQNQ

SEQ ID NO: 957 - protein, 7 aa, synthetic construct
HGSGQNQ

SEQ ID NO: 958 - protein, 7 aa, synthetic construct
NGSGQNQ

SEQ ID NO: 959 - protein, 7 aa, synthetic construct
NGSGQVQ

SEQ ID NO: 960 - protein, 7 aa, synthetic construct
NGPGQNQ

SEQ ID NO: 961 - protein, 7 aa, synthetic construct
NGSGQNQ

SEQ ID NO: 962 - protein, 7 aa, synthetic construct
NGSGQNQ

SEQ ID NO: 963 - protein, 7 aa, synthetic construct
FGSGQNQ

SEQ ID NO: 964 - protein, 7 aa, synthetic construct
NGSGQNQ

SEQ ID NO: 965 - protein, 7 aa, synthetic construct
PGSGQNQ

SEQ ID NO: 966 - protein, 7 aa, synthetic construct
SGSGQNQ

SEQ ID NO: 967 - protein, 7 aa, synthetic construct
NGSGQNQ

SEQ ID NO: 968 - protein, 7 aa, synthetic construct
NGSGQNQ

SEQ ID NO: 969 - protein, 7 aa, synthetic construct
NGSGQNQ

SEQ ID NO: 970 - protein, 7 aa, synthetic construct
NGSGQNQ

SEQ ID NO: 971 - protein, 7 aa, synthetic construct
SGSGQNQ

SEQ ID NO: 972 - protein, 7 aa, synthetic construct
SGSGQNQ

SEQ ID NO: 973 - protein, 7 aa, synthetic construct
NGSGQNQ

SEQ ID NO: 974 - protein, 7 aa, synthetic construct
NGSGQNQ

SEQ ID NO: 975 - protein, 7 aa, synthetic construct
NGSSQNQ

SEQ ID NO: 976 - protein, 7 aa, synthetic construct
NGSGQVQ

SEQ ID NO: 977 - protein, 7 aa, synthetic construct
NGSGQNQ

SEQ ID NO: 978 - protein, 7 aa, synthetic construct
NGSGQNQ

SEQ ID NO: 979 - protein, 7 aa, synthetic construct
NGSTQNQ

SEQ ID NO: 980 - protein, 7 aa, synthetic construct
NGSGQNQ

SEQ ID NO: 981 - protein, 7 aa, synthetic construct
NGSGQNQ

SEQ ID NO: 982 - protein, 7 aa, synthetic construct
IGSGQNQ

SEQ ID NO: 983 - protein, 7 aa, synthetic construct
NGSGQNQ

SEQ ID NO: 984 - protein, 7 aa, synthetic construct
HGSGQNQ

SEQ ID NO: 985 - protein, 7 aa, synthetic construct
NGNGQNQ

SEQ ID NO: 986 - protein, 7 aa, synthetic construct
NGSMQNQ

SEQ ID NO: 987 - protein, 7 aa, synthetic construct
NGSMQNQ

SEQ ID NO: 988 - protein, 7 aa, synthetic construct
NGSMQNQ

SEQ ID NO: 989 - protein, 7 aa, synthetic construct
NGSTQNQ

SEQ ID NO: 990 - protein, 7 aa, synthetic construct
NGSFQNQ

SEQ ID NO: 991 - protein, 7 aa, synthetic construct
NGSGQNQ

SEQ ID NO: 992 - protein, 7 aa, synthetic construct
NGSGQNQ

SEQ ID NO: 993 - protein, 7 aa, synthetic construct
NGSGQNQ

SEQ ID NO: 994 - protein, 7 aa, synthetic construct
NGSGQNQ

SEQ ID NO: 995 - protein, 7 aa, synthetic construct
NGSGQNQ

SEQ ID NO: 996 - protein, 7 aa, synthetic construct
NGSGQNQ

SEQ ID NO: 997 - protein, 7 aa, synthetic construct
NGSGQIQ

SEQ ID NO: 998 - protein, 7 aa, synthetic construct
NGSMQNQ

SEQ ID NO: 999 - protein, 7 aa, synthetic construct
NGSGQFQ

SEQ ID NO: 1000 - protein, 7 aa, synthetic construct
IGSGQNQ

SEQ ID NO: 1001 - protein, 7 aa, synthetic construct
DGSGQNQ

SEQ ID NO: 1002 - protein, 7 aa, synthetic construct
NGSGQNQ

SEQ ID NO: 1003 - protein, 7 aa, synthetic construct
NGSGINQ

SEQ ID NO: 1004 - protein, 7 aa, synthetic construct
NGSNQNQ

SEQ ID NO: 1005 - protein, 7 aa, synthetic construct
NGSGQNQ

SEQ ID NO: 1006 - protein, 7 aa, synthetic construct
NGSGQNQ

SEQ ID NO: 1007 - protein, 7 aa, synthetic construct
NGSGQNQ

SEQ ID NO: 1008 - protein, 7 aa, synthetic construct
FGSGQNQ

SEQ ID NO: 1009 - protein, 7 aa, synthetic construct
NGSGVNQ

SEQ ID NO: 1010 - protein, 7 aa, synthetic construct
NGSGQNQ

SEQ ID NO: 1011 - protein, 7 aa, synthetic construct
NGSGQNQ

SEQ ID NO: 1012 - protein, 7 aa, synthetic construct
NGSGQNQ

SEQ ID NO: 1013 - protein, 7 aa, synthetic construct
WGSGQNQ

SEQ ID NO: 1014 - protein, 7 aa, synthetic construct
NGSGQNQ

SEQ ID NO: 1015 - protein, 7 aa, synthetic construct
NGSGQTQ

SEQ ID NO: 1016 - protein, 7 aa, synthetic construct
VGSGQNQ

SEQ ID NO: 1017 - protein, 7 aa, synthetic construct
NGSGQNQ

SEQ ID NO: 1018 - protein, 7 aa, synthetic construct
NGSLQNQ

SEQ ID NO: 1019 - protein, 7 aa, synthetic construct
IGSGQNQ

SEQ ID NO: 1020 - protein, 7 aa, synthetic construct
NGSGSNQ

SEQ ID NO: 1021 - protein, 7 aa, synthetic construct
NGSGSNQ

SEQ ID NO: 1022 - protein, 7 aa, synthetic construct
NGSGQNQ

SEQ ID NO: 1023 - protein, 7 aa, synthetic construct
NGSGQNQ

SEQ ID NO: 1024 - protein, 7 aa, synthetic construct
NGSGQNP

SEQ ID NO: 1025 - protein, 7 aa, synthetic construct
NGSGQNQ

SEQ ID NO: 1026 - protein, 7 aa, synthetic construct
MGSGQNQ

SEQ ID NO: 1027 - protein, 7 aa, synthetic construct
NGSGQAQ

SEQ ID NO: 1028 - protein, 7 aa, synthetic construct
NGSGQNF

SEQ ID NO: 1029 - protein, 7 aa, synthetic construct
NGSGQNQ

SEQ ID NO: 1030 - protein, 7 aa, synthetic construct
FGSGQNQ

SEQ ID NO: 1031 - protein, 7 aa, synthetic construct
NGSGQNQ

SEQ ID NO: 1032 - protein, 7 aa, synthetic construct
NGSGQNQ

SEQ ID NO: 1033 - protein, 7 aa, synthetic construct
NGSGQNQ

SEQ ID NO: 1034 - protein, 7 aa, synthetic construct
NGSGQNQ

SEQ ID NO: 1035 - protein, 7 aa, synthetic construct
SGSGQNQ

SEQ ID NO: 1036 - protein, 7 aa, synthetic construct
NGSGQNQ

SEQ ID NO: 1037 - protein, 7 aa, synthetic construct
GGSGQNQ

SEQ ID NO: 1038 - protein, 7 aa, synthetic construct
DGSGQNQ

SEQ ID NO: 1039 - protein, 7 aa, synthetic construct
NGSGQNQ

SEQ ID NO: 1040 - protein, 7 aa, synthetic construct
NGSGQNQ

SEQ ID NO: 1041 - protein, 7 aa, synthetic construct
PGSGQNQ

SEQ ID NO: 1042 - protein, 7 aa, synthetic construct
NGSGQNQ

SEQ ID NO: 1043 - protein, 7 aa, synthetic construct
NGSGQNQ

SEQ ID NO: 1044 - protein, 7 aa, synthetic construct
NGSGQNQ

SEQ ID NO: 1045 - protein, 7 aa, synthetic construct
NGSGQNQ

SEQ ID NO: 1046 - protein, 7 aa, synthetic construct
NGSGQNQ

SEQ ID NO: 1047 - protein, 7 aa, synthetic construct
NGSTQNQ

SEQ ID NO: 1048 - protein, 7 aa, synthetic construct
NGSGQNQ

SEQ ID NO: 1049 - protein, 7 aa, synthetic construct
NGSGQNQ

SEQ ID NO: 1050 - protein, 7 aa, synthetic construct
NGSGQNQ

SEQ ID NO: 1051 - protein, 7 aa, synthetic construct
NGSGQNQ

SEQ ID NO: 1052 - protein, 7 aa, synthetic construct
NGSGQNQ

SEQ ID NO: 1053 - protein, 7 aa, synthetic construct
NGSGQNQ

SEQ ID NO: 1054 - protein, 7 aa, synthetic construct
NGSGQNQ

SEQ ID NO: 1055 - protein, 7 aa, synthetic construct
QGSGQNQ

SEQ ID NO: 1056 - protein, 7 aa, synthetic construct
FGSGQNQ

SEQ ID NO: 1057 - protein, 7 aa, synthetic construct
NGSGQNQ

SEQ ID NO: 1058 - protein, 7 aa, synthetic construct
NGSGQNQ

SEQ ID NO: 1059 - protein, 7 aa, synthetic construct
NGSGQNQ

SEQ ID NO: 1060 - protein, 7 aa, synthetic construct
NGSGQNQ

SEQ ID NO: 1061 - protein, 7 aa, synthetic construct
NISGQNQ

SEQ ID NO: 1062 - protein, 7 aa, synthetic construct
NGSGQNQ

SEQ ID NO: 1063 - protein, 7 aa, synthetic construct
NGSGQNQ

SEQ ID NO: 1064 - protein, 7 aa, synthetic construct
NGSGQNQ

SEQ ID NO: 1065 - protein, 7 aa, synthetic construct
NGSGQNQ

SEQ ID NO: 1066 - protein, 7 aa, synthetic construct
NGSGQNQ

SEQ ID NO: 1067 - protein, 7 aa, synthetic construct
NGSGQNQ

SEQ ID NO: 1068 - protein, 7 aa, synthetic construct
NGSGQNQ

SEQ ID NO: 1069 - protein, 7 aa, synthetic construct
NGQGQNQ

SEQ ID NO: 1070 - protein, 7 aa, synthetic construct
NGSGQNQ

SEQ ID NO: 1071 - protein, 7 aa, synthetic construct
NGSGQNQ

SEQ ID NO: 1072 - protein, 7 aa, synthetic construct
NGSGQGQ

SEQ ID NO: 1073 - protein, 7 aa, synthetic construct
NGSGQNQ

SEQ ID NO: 1074 - protein, 7 aa, synthetic construct
NGSGQNQ

SEQ ID NO: 1075 - protein, 7 aa, synthetic construct
NGSGQNQ

SEQ ID NO: 1076 - protein, 7 aa, synthetic construct
NGSGGNQ

SEQ ID NO: 1077 - protein, 7 aa, synthetic construct
NGSGQNQ

SEQ ID NO: 1078 - protein, 7 aa, synthetic construct
NGSGQNQ

SEQ ID NO: 1079 - protein, 7 aa, synthetic construct
NGSGQLQ

SEQ ID NO: 1080 - protein, 7 aa, synthetic construct
NGSGQNQ

SEQ ID NO: 1081 - protein, 7 aa, synthetic construct
NGSGQNQ

SEQ ID NO: 1082 - protein, 7 aa, synthetic construct
NGSGQNQ

SEQ ID NO: 1083 - protein, 7 aa, synthetic construct
NGSGQNQ

SEQ ID NO: 1084 - protein, 7 aa, synthetic construct
MGSGQNQ

SEQ ID NO: 1085 - protein, 7 aa, synthetic construct
MGSGQNQ

SEQ ID NO: 1086 - protein, 7 aa, synthetic construct
NGSAQNQ

SEQ ID NO: 1087 - protein, 7 aa, synthetic construct
NGSGQNQ

SEQ ID NO: 1088 - protein, 7 aa, synthetic construct
NGSGQNQ

SEQ ID NO: 1089 - protein, 7 aa, synthetic construct
NGSGQNQ

SEQ ID NO: 1090 - protein, 7 aa, synthetic construct
PGSGQNQ

SEQ ID NO: 1091 - protein, 7 aa, synthetic construct
NNSGQNQ

SEQ ID NO: 1092 - protein, 7 aa, synthetic construct
NESGQNQ

SEQ ID NO: 1093 - protein, 7 aa, synthetic construct
NGSGQNQ

SEQ ID NO: 1094 - protein, 7 aa, synthetic construct
NGSGQFQ

SEQ ID NO: 1095 - protein, 7 aa, synthetic construct
NGSAQNQ

SEQ ID NO: 1096 - protein, 7 aa, synthetic construct
NGSGQNQ

SEQ ID NO: 1097 - protein, 7 aa, synthetic construct
NGSGQNQ

SEQ ID NO: 1098 - protein, 7 aa, synthetic construct
NGSLQNQ

SEQ ID NO: 1099 - protein, 7 aa, synthetic construct
NGSGQNQ

SEQ ID NO: 1100 - protein, 7 aa, synthetic construct
NGSGQNQ

SEQ ID NO: 1101 - protein, 7 aa, synthetic construct
NGSGQNQ

SEQ ID NO: 1102 - protein, 7 aa, synthetic construct
NGSGQNQ

SEQ ID NO: 1103 - protein, 7 aa, synthetic construct
NGSGQNQ

SEQ ID NO: 1104 - protein, 7 aa, synthetic construct
NGSGQNQ

SEQ ID NO: 1105 - protein, 7 aa, synthetic construct
NGSGQNQ

SEQ ID NO: 1106 - protein, 7 aa, synthetic construct
NGSGQNQ

SEQ ID NO: 1107 - protein, 7 aa, synthetic construct
DGSGQNQ

SEQ ID NO: 1108 - protein, 7 aa, synthetic construct
NGSGQNQ

SEQ ID NO: 1109 - protein, 7 aa, synthetic construct
NGSGQNQ

SEQ ID NO: 1110 - protein, 7 aa, synthetic construct
LGSGQNQ

SEQ ID NO: 1111 - protein, 7 aa, synthetic construct
NGSGQNQ

SEQ ID NO: 1112 - protein, 7 aa, synthetic construct
NGSTQNQ

SEQ ID NO: 1113 - protein, 7 aa, synthetic construct
NGSGQNQ

SEQ ID NO: 1114 - protein, 7 aa, synthetic construct
GGSGQNQ

SEQ ID NO: 1115 - protein, 7 aa, synthetic construct
NGSGQNQ

SEQ ID NO: 1116 - protein, 7 aa, synthetic construct
TGSGQNQ

SEQ ID NO: 1117 - protein, 7 aa, synthetic construct
TGSGQNQ

SEQ ID NO: 1118 - protein, 7 aa, synthetic construct
NGSGQNQ

SEQ ID NO: 1119 - protein, 7 aa, synthetic construct
NGSGQNQ

SEQ ID NO: 1120 - protein, 7 aa, synthetic construct
GGSGQNQ

SEQ ID NO: 1121 - protein, 7 aa, synthetic construct
NGSGQNQ

SEQ ID NO: 1122 - protein, 7 aa, synthetic construct
NGSGQNQ

SEQ ID NO: 1123 - protein, 7 aa, synthetic construct
NGSGQNQ

SEQ ID NO: 1124 - protein, 7 aa, synthetic construct
NGSGQNQ

SEQ ID NO: 1125 - protein, 7 aa, synthetic construct
NGSGQNQ

SEQ ID NO: 1126 - protein, 7 aa, synthetic construct
NGSGQNQ

SEQ ID NO: 1127 - protein, 7 aa, synthetic construct
NGSGQNQ

SEQ ID NO: 1128 - protein, 7 aa, synthetic construct
TGSGQNQ

SEQ ID NO: 1129 - protein, 7 aa, synthetic construct
NGSGQNQ

SEQ ID NO: 1130 - protein, 7 aa, synthetic construct
NGSGQNQ

SEQ ID NO: 1131 - protein, 7 aa, synthetic construct
NGHGQNQ

SEQ ID NO: 1132 - protein, 7 aa, synthetic construct
NGSGQNQ

SEQ ID NO: 1133 - protein, 7 aa, synthetic construct
NGSGQNQ

SEQ ID NO: 1134 - protein, 7 aa, synthetic construct
NGSGQNQ

SEQ ID NO: 1135 - protein, 7 aa, synthetic construct
NGSIQNQ

SEQ ID NO: 1136 - protein, 7 aa, synthetic construct
NVSGQNQ

SEQ ID NO: 1137 - protein, 7 aa, synthetic construct
NGSGQNQ

SEQ ID NO: 1138 - protein, 7 aa, synthetic construct
NGSGQNQ

SEQ ID NO: 1139 - protein, 7 aa, synthetic construct
DGSGQNQ

SEQ ID NO: 1140 - protein, 7 aa, synthetic construct
NGSGQNQ

SEQ ID NO: 1141 - protein, 7 aa, synthetic construct
NGSGQNQ

SEQ ID NO: 1142 - protein, 7 aa, synthetic construct
IGSGQNQ

SEQ ID NO: 1143 - protein, 7 aa, synthetic construct
NGSGQNQ

SEQ ID NO: 1144 - protein, 7 aa, synthetic construct
NGSGQNQ

SEQ ID NO: 1145 - protein, 7 aa, synthetic construct
NGSGQNQ

SEQ ID NO: 1146 - protein, 7 aa, synthetic construct
NGSGQNQ

SEQ ID NO: 1147 - protein, 7 aa, synthetic construct
NGSGQNQ

SEQ ID NO: 1148 - protein, 7 aa, synthetic construct
NGSGQNQ

SEQ ID NO: 1149 - protein, 7 aa, synthetic construct
NGSGQFQ

SEQ ID NO: 1150 - protein, 7 aa, synthetic construct
NGSGQNQ

SEQ ID NO: 1151 - protein, 7 aa, synthetic construct
NGSSQNQ

SEQ ID NO: 1152 - protein, 7 aa, synthetic construct
NGSGQNQ

SEQ ID NO: 1153 - protein, 7 aa, synthetic construct
NGSGQNQ

SEQ ID NO: 1154 - protein, 7 aa, synthetic construct
NGSGQNQ

SEQ ID NO: 1155 - protein, 7 aa, synthetic construct
NGIGQNQ

SEQ ID NO: 1156 - protein, 7 aa, synthetic construct
NGSGQNQ

SEQ ID NO: 1157 - protein, 7 aa, synthetic construct
NGSGQNQ

SEQ ID NO: 1158 - protein, 7 aa, synthetic construct
NGSGQNQ

SEQ ID NO: 1159 - protein, 7 aa, synthetic construct
NGSGQNQ

SEQ ID NO: 1160 - protein, 7 aa, synthetic construct
NGSGQNQ

SEQ ID NO: 1161 - protein, 7 aa, synthetic construct
NGSGQNQ

SEQ ID NO: 1162 - protein, 7 aa, synthetic construct
NGSGQNQ

SEQ ID NO: 1163 - protein, 7 aa, synthetic construct
NGSGQNQ

SEQ ID NO: 1164 - protein, 7 aa, synthetic construct
NGSGQNQ

SEQ ID NO: 1165 - protein, 7 aa, synthetic construct
LGSGQNQ

SEQ ID NO: 1166 - protein, 7 aa, synthetic construct
NGSGQNQ

SEQ ID NO: 1167 - protein, 7 aa, synthetic construct
NGSGQNQ

SEQ ID NO: 1168 - protein, 7 aa, synthetic construct
NGSGQNQ

SEQ ID NO: 1169 - protein, 7 aa, synthetic construct
NGHGQNQ

SEQ ID NO: 1170 - protein, 7 aa, synthetic construct
FGSGQNQ

SEQ ID NO: 1171 - protein, 7 aa, synthetic construct
NGSGQNQ

SEQ ID NO: 1172 - protein, 7 aa, synthetic construct
NGSGQNQ

SEQ ID NO: 1173 - protein, 7 aa, synthetic construct
NGSGQNQ

SEQ ID NO: 1174 - protein, 7 aa, synthetic construct
NGSTQNQ

SEQ ID NO: 1175 - protein, 7 aa, synthetic construct
NGSGQNQ

SEQ ID NO: 1176 - protein, 7 aa, synthetic construct
NGSGQNQ

SEQ ID NO: 1177 - protein, 7 aa, synthetic construct
NGSGQNQ

SEQ ID NO: 1178 - protein, 7 aa, synthetic construct
NGSGQNQ

SEQ ID NO: 1179 - protein, 7 aa, synthetic construct
NGSGQNQ

SEQ ID NO: 1180 - protein, 7 aa, synthetic construct
NGSGQNQ

SEQ ID NO: 1181 - protein, 7 aa, synthetic construct
NGSGQNQ

SEQ ID NO: 1182 - protein, 7 aa, synthetic construct
NGSGQNQ

SEQ ID NO: 1183 - protein, 7 aa, synthetic construct
NGSIQNQ

SEQ ID NO: 1184 - protein, 7 aa, synthetic construct
NGSGQNQ

SEQ ID NO: 1185 - protein, 7 aa, synthetic construct
NGSIQNQ

SEQ ID NO: 1186 - protein, 7 aa, synthetic construct
NGSGQNQ

SEQ ID NO: 1187 - protein, 7 aa, synthetic construct
NGSGQNQ

SEQ ID NO: 1188 - protein, 7 aa, synthetic construct
NGSGQNQ

SEQ ID NO: 1189 - protein, 7 aa, synthetic construct
NGSGQNQ

SEQ ID NO: 1190 - protein, 7 aa, synthetic construct
NGSGQNQ

SEQ ID NO: 1191 - protein, 7 aa, synthetic construct
NGSGQNQ

SEQ ID NO: 1192 - protein, 7 aa, synthetic construct
NGSGQNQ

SEQ ID NO: 1193 - protein, 7 aa, synthetic construct
NGSGQHQ

SEQ ID NO: 1194 - protein, 7 aa, synthetic construct
NGSGQNQ

SEQ ID NO: 1195 - protein, 7 aa, synthetic construct
NGSGQNQ

SEQ ID NO: 1196 - protein, 7 aa, synthetic construct
CGSGQNQ

SEQ ID NO: 1197 - protein, 7 aa, synthetic construct
NGSGQNQ

SEQ ID NO: 1198 - protein, 7 aa, synthetic construct
NGSGQNQ

SEQ ID NO: 1199 - protein, 7 aa, synthetic construct
NGSGQNQ

SEQ ID NO: 1200 - protein, 7 aa, synthetic construct
NGSGQNQ

SEQ ID NO: 1201 - protein, 7 aa, synthetic construct
NGSGQNQ

SEQ ID NO: 1202 - protein, 7 aa, synthetic construct
NGSGQNQ

SEQ ID NO: 1203 - protein, 7 aa, synthetic construct
NGSGQNQ

SEQ ID NO: 1204 - protein, 7 aa, synthetic construct
NGSGQNQ

SEQ ID NO: 1205 - protein, 7 aa, synthetic construct
NGSGQNQ

SEQ ID NO: 1206 - protein, 7 aa, synthetic construct
NGSTQNQ

SEQ ID NO: 1207 - protein, 7 aa, synthetic construct
NGSGQNN

SEQ ID NO: 1208 - protein, 7 aa, synthetic construct
NGSGQNQ

SEQ ID NO: 1209 - protein, 7 aa, synthetic construct
NGSGQNQ

SEQ ID NO: 1210 - protein, 7 aa, synthetic construct
NGSGQNQ

SEQ ID NO: 1211 - protein, 7 aa, synthetic construct
NGSGQNQ

SEQ ID NO: 1212 - protein, 7 aa, synthetic construct
NGSGQNQ

SEQ ID NO: 1213 - protein, 7 aa, synthetic construct
NGSGQNQ

SEQ ID NO: 1214 - protein, 7 aa, synthetic construct
NGSGQNQ

SEQ ID NO: 1215 - protein, 7 aa, synthetic construct
NGSGQNQ

SEQ ID NO: 1216 - protein, 7 aa, synthetic construct
NGSPQNQ

SEQ ID NO: 1217 - protein, 7 aa, synthetic construct
NGSGQNQ

SEQ ID NO: 1218 - protein, 7 aa, synthetic construct
NGSGQNQ

SEQ ID NO: 1219 - protein, 7 aa, synthetic construct
NGSGQNQ

SEQ ID NO: 1220 - protein, 7 aa, synthetic construct
NGSGQNQ

SEQ ID NO: 1221 - protein, 7 aa, synthetic construct
NGSGQNQ

SEQ ID NO: 1222 - protein, 7 aa, synthetic construct
NGSGQNQ

SEQ ID NO: 1223 - protein, 7 aa, synthetic construct
NGSGQNQ

SEQ ID NO: 1224 - protein, 7 aa, synthetic construct
NGSGQNQ

SEQ ID NO: 1225 - protein, 7 aa, synthetic construct
NGSGQNQ

SEQ ID NO: 1226 - protein, 7 aa, synthetic construct
NGSGQNQ

SEQ ID NO: 1227 - protein, 7 aa, synthetic construct
NGSGQNQ

SEQ ID NO: 1228 - protein, 7 aa, synthetic construct
NGSGQNQ

SEQ ID NO: 1229 - protein, 7 aa, synthetic construct
NGSGQNQ

SEQ ID NO: 1230 - protein, 7 aa, synthetic construct
NGSGQNQ

SEQ ID NO: 1231 - protein, 7 aa, synthetic construct
NGSGQNQ

SEQ ID NO: 1232 - protein, 7 aa, synthetic construct
NGSGQNQ

SEQ ID NO: 1233 - protein, 7 aa, synthetic construct
NGSGQNQ

SEQ ID NO: 1234 - protein, 7 aa, synthetic construct
NGSGQNQ

SEQ ID NO: 1235 - protein, 7 aa, synthetic construct
NGSGQNQ

SEQ ID NO: 1236 - protein, 7 aa, synthetic construct
NGSGQNQ

SEQ ID NO: 1237 - protein, 7 aa, synthetic construct
NGSGQNQ

SEQ ID NO: 1238 - protein, 7 aa, synthetic construct
NGSGQNQ

SEQ ID NO: 1239 - protein, 7 aa, synthetic construct
NGSGQNQ

SEQ ID NO: 1240 - protein, 7 aa, synthetic construct
MGSGQNQ

SEQ ID NO: 1241 - protein, 7 aa, synthetic construct
NGSGQNQ

SEQ ID NO: 1242 - protein, 7 aa, synthetic construct
NGSGQNV

SEQ ID NO: 1243 - protein, 7 aa, synthetic construct
NGSGGNQ

SEQ ID NO: 1244 - protein, 7 aa, synthetic construct
NGSGQDQ

SEQ ID NO: 1245 - protein, 7 aa, synthetic construct
NGSGQNQ

SEQ ID NO: 1246 - protein, 7 aa, synthetic construct
NGSGQNQ

SEQ ID NO: 1247 - protein, 7 aa, synthetic construct
NGSGQNQ

SEQ ID NO: 1248 - protein, 7 aa, synthetic construct
NGNGQNQ

SEQ ID NO: 1249 - protein, 7 aa, synthetic construct
NGSGQNQ

SEQ ID NO: 1250 - protein, 7 aa, synthetic construct
NGSGQNQ

SEQ ID NO: 1251 - protein, 7 aa, synthetic construct
NGSGQNQ

SEQ ID NO: 1252 - protein, 7 aa, synthetic construct
NGSGQNQ

SEQ ID NO: 1253 - protein, 7 aa, synthetic construct
NGSGQNQ

SEQ ID NO: 1254 - protein, 7 aa, synthetic construct
NGSGQNQ

SEQ ID NO: 1255 - protein, 7 aa, synthetic construct
NGSGQNQ

SEQ ID NO: 1256 - protein, 7 aa, synthetic construct
NGSGQNQ

SEQ ID NO: 1257 - protein, 7 aa, synthetic construct
NGSGQNQ

SEQ ID NO: 1258 - protein, 7 aa, synthetic construct
NGSGQNQ

SEQ ID NO: 1259 - protein, 7 aa, synthetic construct
NGSGQNQ

SEQ ID NO: 1260 - protein, 7 aa, synthetic construct
NGSGQNQ

SEQ ID NO: 1261 - protein, 7 aa, synthetic construct
NGSGQNQ

SEQ ID NO: 1262 - protein, 7 aa, synthetic construct
NGSGQNQ

SEQ ID NO: 1263 - protein, 7 aa, synthetic construct
NGSNQNQ

SEQ ID NO: 1264 - protein, 7 aa, synthetic construct
NGSGQNQ

SEQ ID NO: 1265 - protein, 7 aa, synthetic construct
NGSGQNQ

SEQ ID NO: 1266 - protein, 7 aa, synthetic construct
NGSGQNQ

SEQ ID NO: 1267 - protein, 7 aa, synthetic construct
NGSGQNQ

SEQ ID NO: 1268 - protein, 7 aa, synthetic construct
NGSGQNQ

SEQ ID NO: 1269 - protein, 7 aa, synthetic construct
NGSGQNQ

SEQ ID NO: 1270 - protein, 7 aa, synthetic construct
NGSGQNQ

SEQ ID NO: 1271 - protein, 7 aa, synthetic construct
NGSGQNQ

SEQ ID NO: 1272 - protein, 7 aa, synthetic construct
NGSMQNQ

SEQ ID NO: 1273 - protein, 7 aa, synthetic construct
NGSGQNQ